SEQUENCE LISTING (50 sequences)

SEQ ID NO: 1 - DNA, 71138 bp, Nonomuria
  misc_feature (40)..(1140): ORF1 negative strandedness
gggggctgggcctgctgcggctcgcgagcgggctgacggtcaggagacgaaccccgcgccggggcgggtcgtcctgagtgcctgggctgcggcgacgtcgccgcagcctgccaggccgagcccgtcctcgatctcggcacccaggaggccgagcaccgtacggaccccccgttcgccgtccgcggccagaccccagatcacggggcgtccgacgagcacacccgacgccccgagcgccagcgccttgaggacgtcggctcccgaccggacgccaccatcgagcatgatctcgcagcggcccccgacgctctccgccacccccggcagcgcgtcgagactggccacggcgccgtcgagctgacgtccgccgtggttggagaccacgatgccgtcgatgccgaggtccgcggcgcggcgggcgtcctcggggtgcagaatgcccttgaccaccagcgggagcccgctggcggcccggagggtctcgaggtacgaccagtccaccgcggcggagagctccatggccgtgtgcgccgccagcgcggagccgccggaggcaccccgatgagcctcggtcccggagttcgccgtcaggtgcacgggccgcacgtgcgggggcaggcggaaccggttgcggatgtcacgtggcctgcggcccatccacggcacatcgagcgtgagcatcaacgcccggcaccccgcgtcctcggcccggcggatcaggccgagggtggcggcgtgctcgcgaaggcagtagagctggaaccagacgtgtccccccagggcggtgacgtcctccaccgggacgctgctcaaggtgctgacggtgaacgggaccccggcgtcccgcgccgcccgggccgtcgccagctcaccgtcgggatgcacgagccggtggtaggcgacgggggccaccgccaccggcatcgtcgcggggtggcccagcagcgtcgcacgggtggagcacgccgacacgtcctggagcacccgcggcaccaggaacacccggtcgaaggcggcccgattcgcacggagggtctgctcgcggccgctcccgccgtcgatgaagtcccggacgtcggcggggaggaccttggcggcgatctcctcgtactcggcgaggcagacgggactttcgtgcacgctgtcaggacgctcgggcccgctgccgggacgctcgggcccgctgccgggacgctcgggcccgctgccgggacgctcgggcccgctgccgggacgctcccgcacgctgctgggacgctcatgcacgctgctgggacctcgccacctcgacggcctcgtagagggccttgatgttggcgcctccgaaggtgcgggctccctgccgctcgatgacctcgaagaagagggtctcgcgcggatgggtggacgccgtgaagatctggaagagctgtccgccgtgatcctcgtcagcgagcagtcccgtcgcgcgcaactggtccaccgtgtgaccccggatctggatccgtgattcgagcaggtcgtagtagctgcccggcgtgctgaggaagcggacgccccgctcggacagggtgttcacggcgtgcacggcgtccgaggaggagaaggcgacgtgctgcaccccggcaccggcgtgccgttcgaggaacatgtcgatctggccggcctcggccatcgggtcgggttcgatgagtgtcagcgtgacggcgccggaggcgctctgcaccaccttggactccatggcctgggtgccgacctcgatgcgttccttgaaggtctcgctgaagccgagggtggcgacgtagaagtcggtgatgatgtcgaggtcacccgtgggcaggcacacggcgaagtggtcgatgtcgagcagctccgccgcgtccgcaccggactcggcggcggacggagcctcggagaagccgaccggcaggccggggtcgtcgccggggtcccgctggacgagggtgtggaccacgtcgccgaagccgccgatcgcggcggagcaggccggcccggggccggggtgccgggacggggaccgtacgggccgggcgccggcggccacggcatgggtgaagacgacgtcgacgtcgggggtccgcagggcgatgtcggcgaccccgtcgccgtgcgtccgcacataggccgaagccggatggccgtcggacgtggcctgggtgaggacgagggtgatgcggccctgccggagcgcgacgctgcgatggtcgctggcgttcgccgtgcccacgacggcgaagcggtattcgtcggtccaagggagagtggcgaccttcagatccgctacgtacatttcgacgtaatcaacggcgagaggcggaagcgattccatattccgacgctacggccgggcggggaggttcgcaccgtgtccattggacgcgctcgcaggccgcgctcacagcagattccggtacattcccgaggccttttcaggccggcgtggacggtcggcggatcaggcttcataaaaagcctgccctggcgtattctcgggttaatcaaccccgatggatatcctgcccgaggccggcgaattcggcttgtcgaactcttcgctctacagccgcactgcctcacgcggctcgcggccggctgtggccgtcgccttatcggcgatgtccgcggcgaagaggttgcccaggtcaccccgagtctgtacgtggagtttccgatagatcctggtgagatgctgctcgaccgtgctgcgcgtgatgtagagcgcctcggcgatctcacgattggtgtgcccacgggcggcgagcacggcgacccgccgttcggcgccgctcaacggtgcggtctcaccatgatcgtgctgtgcggcgagcctcctcatcaagggcttcgcgttgcactcgcgggccagctcctgcgcccgcacccagtaggcccgggcctcgtccttgccgcccttgagctgtggggtcccggcgaggtcgcagagggaaagggccagctggtagcgatcctgggcggcctccagcgcgtccacggactgcatcagcaaccgctggcgctgtgcgggcttgctcagctgcgcgtgcaggcggagggcgaccccgtacgtccgcaggtcaccggaggaggtgtgggcgatctgggccgtgaccagatcggccgccctgcgacgccagcccagttgcaggcacgctcgcgccgcgccgaggcgccaggggaccacgtcggacaggctgctgccccagcgctggaccgcctgcccgcatgccaggaagccggcgaaggcggcgcgaggctgctccgtgaccaggtggtagtgcgccctcgcgagctcgtagccgatcccgaaggcggtctccgccgtctcgcggggcatcggcaccgccacggtcgccttcgcctcgtcgaggtggcccatcgcggtctgggcatggagcagggtgctgagtggcgcgccgatcgcgacgccccagccgctgggctgcagtatggtcagcgcctcctgcgcatgggcctcggcccccgccaggtcgcccttgcgccacgccgtctccgcccggatggcggagatgatcgccttccaggtgggcgccttcgtcaccccgggctccttgaggagcgtctcgcaggaggccgccacctccgacactccgcccagcagcagtgccatcagagccgagatgatgctgtccatcgcctcatccgtcggctccgcctgacgcaggatccggcgcgcgtcctcgactgtctggcccatcgagccgcgggcggaccggggcaatctgtcgagaagcaccgggtggacgtggcacatcgcgatcaaggacgcgtcggcgtccctgtcggcgacgctcggcctcagccgatcgatcagctccgccgcgtcggcgaaccggccgtaccacagcagctggcggaacagctccatcccgtgagatccacgcaacgcacccgagcgcgtggcgtcgagcagatcgggcacgtgacgtgccgccactgccgggtcgacgcgccactccgccgcggcgagcatcaccttcacgtccagccggcgaggcgtgccccagccggacgccagggcgagccgtaagcacttcatgacagcgacgaaatcaccctcgtcgaacgcctgccttcccgcttcgacgaggacgtcgaaagcccactcctcacccgaccagcccgcctccagcaagcgcgtggccacagccgacggtgggcctccccgccgatggaggatctccgcggcccgccggaggatctccatcctgccgtaggaggtcatgcgcccgagtacggccaggcggcccgcctcatgacgaaagcgccccccggccaggagcccggcgcgctccagcatccccatcgagcgtgtcgcggcaggaggggcgatgcccacgagctcgcccaccgcgtccggcgtggcgtgctcgccgaggacggccaccgcctccgcgacgcggacggcctccggctcgcagccatgaacgcacgctgccaccgcgctcatgaaggagtcgccgaccacgagcccgggcgcgccggcctcctgatcctcgatcagcgcccggaccagcagcgggctgccgccgctgaagcggtagaggtcgtcggcgagctggtcggccgtctcggccccgaggtgctcggtggcgaactgcctgaccgccggacggggcaggagcggcagctcgaccagctcgatgccgggcaggcgcagcaaggactcggcgacatgggggagaggggccggcggccggtcctggcagatcgtgacggcgatcatcatcctggtgtccgtcagcaggggcgtcatggacaggatggccagcagggacggatcgtcggcgagatccacgtcgtcgatcgtcaggaggatcgggttcgcctcggccatctggaacagggagacgcagacgtcctcgaccgcgtcaccgcccctgcgcgcggtcagccgctccgctgccccgcggcccaggatctggtcggcgaccccccagtccagcgactgctccgccggcgtgcagcgggccgtgaccagccggatgccggccgcgatggatcgcatgccgagctcgtggaggatggcggtcttgccgccgaccacaggccccgtgatgacggccactccaccgcggccggccgcggtggaatcgagcaacctcgtcagactcttcagttcacgatctcgcccgaacagcactcttgctcgtcccccaagcggttcgtcgacttggtttgccgtgtgcctgatctggtcctggtcccgtcgctctacatacggccgcccggctcatccactcgtgctcatccagcggccagatcggtcgcccgcccctccaggcgatccgcgaacgctgcccagatctgggcatgatccctggcgcatctcgcgacaacggtgccccaatgcggcggcactccgcgcaagatcctttcccattcctgcccgtcgatggagtgcaatgatagcattcgcaacaaaattcggccggtttcggtcagacgtaatgcgggatcggccttgagcctttccagcacggcttggcggtctcggccgtcgacgtggccgaaatcctgttctgcccgacgcagtaattccggcttggtccttaatctccggctcccgtccggaatcgggctctcaccgtgctccaaccggcctctcacatcccggaccgtctccggggagatgccgacctgtttggcgacctggcggagcgaaaggtccggatggctgcggatgagctcggcggcgagtctcctcccctctgagctgtccaccggacggatgcgcccgtcccgcccgattctggcctcgtcaccgccttgtcctctgcgcctgcgcaggtcggccaccgtgccggcggagatgccggtcgccgaggccacccgtcgatcagaccactgcggatgtgtcccgatgatccggacggccgcacgcttgcggtcggccagtgagagcggcagcccgtgccgcacgttcgcctcgacggccaggacgaaggcgtccgattcggtgccgtcgatcagcctcaccgagattgttgtttcacccctgacccgcgccaccttcaaccggtgcaggccgtcgatcacccgcatcgttggacggtggacgagaatgggcggaagctccccttgtgccgacaacagggtctcgacgtgctctggatcctcgcccgaagtccggggtgagtacacagaggacagccgggacagctcgatttcgacgacagggagagtggctatgtcaactcccgtcgggtccacctagcctccgattcgattagcgtcatatcggagccgggggcgttcaaaaaacaacccagccgcgtgcgccgcgcgcaccttcgacgattccccgtcgcgcctgcagcatctggtcccgggcaagcctggacttcccggcgcgagctgcataaatcgatcggccaagtgctctgtcgagagaatgcgtcgcatcctcttttcttcggcaactccacgcggcaaagaattggacgctgtcgccgcgaatccgtagccgtctacctcatcgaattgcagaacgcttcccgttagcattccgatcactccgactttcggttaggccttcctccggaaggttaaaggaggctgtgcaggtcgaaccaccccctatcccggacatccacccccctagtttcggataagaccgatgcgcggggttgcgcctctgtcgcgaagcggagtatccggtgctggaccgcccgaatcgagcggtcaccatgcgtgtcaatccgtgtgtattggcatgcgccgtcggcgcgagcccggcggggccgcggcggtcccacggtttcgctcatgacaccgtctccaggtgagggtgatcgcggtagccggccacgggcgcgctgccgcagcggcggccatgctgatctgcccatggaccagcaggccgacggtgaggtcccgccggacggccggctcccggtacaacgtacgtcagttcttctcggcgatctaggggagtgggcggggtgccttcgccgggcatgcgggcggcctgtcctttggcaattgacaggcgtgaatgcagaaaggagcgcggccacctctgacctgccgagtaagggaatggattactcatcaatggcgccggtggccacggaactcgcccggcgatccggcgtgtccaaatggcgcggtgcccaggcccgccgatggacaccgcccggtgcgcgggcttaagaagtagccgtgaccctggagaggacgctcatcgtcggcaccggtctgatcggcacctccgccgcgctcgcccttcgcgagaagggggtggcggtctacctgtccgacgtcgacgcacatgccgtacggctggcgcgagcgctcggcgcgggccaggagtggaccggtcagcgcgtggacctggcattgatcgccgtgcccccgcccagcgtggggcagcggctggccgatctgcagcagcggcgggccgcgcgggcgtacaccgatgtgaccagcgtcaaggtcgatccgatcgccgacgcggagcggctcggctgcgacctgacctcctatgtccccggacacccgctcgccggccgggagcggtccggcccggccgccgcccgtgccgatctgttcctgggacgtccctgggcgctctgcccccgccctgagacgggtgcggatgccgtgcggctggccagggagctggtctcgatgtgcggggcggagccctacaccgtgagtgcgggcgagcacgacacggcggtggcgctggtgtcgcacgccccgcacgtggccgcgtccgcggtggcggcgcggctgagggacggcgacgacgtcgcgctggccctggcggggcaggggctgcgcgacgtgacgcggatcgccgcaggggaccccctgctgtggcggatgattctcgccgcgaacgccctgccggtggccggggtgctggagcggatcgcggccgatctcgccgcggcggcctcggcgctgcggtccggcgatctcgacgatgtgacggatctgctgcggcgcggcgtggacggccacggccggatccccgacaagcacggcggcccggcgcgtgactacacggtgatccaggtggtgctgcaggatcggccgggagagctggcgaggctcttcaacgcggcggggctcgcggacgtcaacatcgaggacatccgcctggagcactcggccggcctgccggtcggggtggtcgaggtctccgtgcgcccggaggacaccggccggctcaccgaggcactgcgcttccacggctggcacgtcccgcccgtccccgacggcaactcgaggatcgaccggacgcgagctatggtgtcagactgacagcccccgatcgagacggcgacacgaatgcgcgttctggtggtggaggaccaagtcgacctggccgactcggtggcgcgggtgctgcgtcgcgagggcatggccgtcgatgtcagtcatgacggcgatgacgcacaggagcgcctctccgtgatcgactacgacgtcgtggtgcttgatcgggatattcccggcgtccatggcgacgagctgtgcgctgagatcgccgtggacgatcgcaggacccgggtgctgatgctcaccgcgtccgggacgaccgctgaccgggtggcgggcctgagcctgggcgccgacgactatctgccgaagccgttcgccttcgccgagctggtggcgcgcatccgcgccctgggcaggcgcgcgcatcctcccgcgccgccgatcctggtccacggcgacctgcggctcgatccggcgcaacgggtggcgatcaggggcggcatgcggctgccgctgaccaccaaggagctggcggtcctggagcatctgctgaccgcgcgcggccgggtggtgtcggccgaggagctgctcgaacgggtctgggacgagcaagccgacccgttcaccaccaccgtgaaggcgacgatcaaccggctgcgctcgaagctcggccagccgccggtgatcgaaaccgtcccgcgcgagggatatcgcatctgatccgcgcggtcacagagcggtcacacgttctctgaccctcgtgtcaccttctgctccgtagaactggtgtcagatcaccagactggaggagagggatgaggagaagcgagggtgacgacgaaccacgcactctcccgcctcgggcccgggaccgggtgtacaccgcggtcacgcgggtgctcgccgtgctcctgctgcccgtggcgttcgtccgtcagcccggccgcgcccgcgagctggcctgcggctgggcgttgaggatgcgattcccggcagaggacctcaccgggctcaccgacggcgccagggcggcgttcaccgcggcgcgggccgaggcgctgtggcgtcacggccagctcgtcggtctcacttccggataccgcgatccccgggtccagcagcggatgttcgaggaggaggtgcgccgctcagggtccgtggccgccgcacggatgttcgtggcgccgccggccgagtccaaccacgtcaagggcatggcgctggacgtacgcccgcacgagggcgcgcgctggctggaggcgcacggcgcccgctacgacctctaccgcatctacgacaacgagtggtggcacttcgaacaccgcccggagtgcggtggcacgccaccacggcggctaccccacccaggcgcggcctgggcgagccggaacgggggccgggtctagctagggcacggggtcgccgcggggatcggtccccggccggcttcggcgctagggcagctcgatgcggccgctccgctgataccagtgacggcccgccagcaaatgggtgacgaccgccttctccagcgtcgagcgctgcggaagctcctccagcggctggccgttgtagcggaagacgaactccaggatcgcgtcgtccccgtcctgcggttcggagtcgaggagcgtccagccgcgtctgagctgcaggatgtgggagtcctcggcgagatactcggccagctgcggatcgagcagccacgaggtgcctgtcgccgcccgggcgccgtgctcgggaaaatgccgttcgaagaacgggcgggcacgacggagcgagtcgtaatagatgtcggggatcagcggcccgcccacttcggggatgtgcaggccgaggacgggcgtgccgtccttggcgacggccaggttgtactggagccggccgagccggtagaccaggccgcgcacgagcagggtgagccaccacggcatgttcgtgccgccctcgccgtacttgcggcgatggatggccaccgattcccccagctgcgtcagggtctcccaggtggtcgcctcggggatgtcccgtgtcgcgtggaagcgccgcaacgccggaagcgtcgcgaggaagacgtacacgtggaagtagcgggcggcggccccggtctcgtacggcagggtcggcccaccccgtaccttcaccttgtagtcgcccatgtgccggacgagctcgtggtgggcgcgttcgagcagccaccacagggccgggtcgcgatccgggccgggggtggccgccacgatctcctcgacgtcgggagccggcacctccagccggtgtaagagatcacgagcctcatcgccctgaggcaggcgcaccggctcggggggcggtccgagctcctcgagccgggagagccacgccgtggcgttctctcccagccgcagctgcctgcgcacgctctcagcatccatcgtcactccgttctgttccgcccggccccggcggccgtgtcgagcaggagtttcgcggccacggccgccccgtcggcgcggatcttcccggccacgtcgatcgcccgcgcgcgggtctcgggagccagcgccgtggtgagcgcggccgacaggctctccacggtcggcacccgcccgtcgtgtgccacgccgatgcccagctcggccacccggccggcgtggtacggctggtcggtcatctggggcaccacgacctggggagcgcccgcccgggtgaccgcggtcgtgatgcccgcgctgccggcgtggacgacggcggccacccggccgaacaacacctggtggttcacctcgccgacggtcaggcagtcgctccggtcgtcgggcggggctaggccggcccagccacgggagacgatcacccggtggccatgggcccggatcgcctcgatggccaccctcgcggcgtcggtgggggcgggcccgctgccgaactccacgtgcaccggtggcgggccggcctccaggaacgcctccacctcggcgggcaggggccgttcgtcgggcatgatccacgcaccggtctgcacgacgtcgaggtccgtccgctgcagcggggccaggaccgggtccgcggccaggaaggggcgatcggtgtagccgtagctgaagatgtcgtccaccggcggcaggccgatcgaggcccgccggctgttgagcgcggcaccgaaccgctggtaggcgccctggttgttgcggtcccacagcacccggttgtcggtcacgtcccgcgcgggctgctcaccgaggggtggcggcggcgggtagtacggcgacggcacatagatggggcagtagaagacgtagacgtaggggatgccgagcttctcggccaccgaccggacggcgaccgccgccgacagcacgccgctcaccaccatcacctcgcacccctcggcggccggcaggacctggtcgagctgcgtggcgatggcctcggcgtcgagccggggcacgtcctcgagcgagggcggcctcttcccgtgcagcttcgcgcgcatcgaggtgccgaccggcaccagcggcaccccggcctcggccagtctctccgcgcagtccggcggggcgcacatccgtgtctccgcgcctagctcacgcagctggaccgccaggcccagcagcggttcgacgtccccgcgtgatccggacgtcgacaacaacacgcgcatgtcgtatccctgttccgtggattctggtgcggatcgatcggaaggccggagcctcaggggtgatgtgtcagccatctcatcccgtcgggtgaggtcaccagcccgccggggaacagtggctgctcgggctcggcgtccgcaccgagcaccgccctcatctgctcctgcccgccctcctgcatcacctgtttgaccacctgtgacttgaacagcggcaccatgctggagtcgtcgccgtcggccatctgatcgacggccgcggcgaactcggcgctgcgctcggcgatccgccctgaggtcgccagcgccgtctcgccggaggacagaccgcccaccaggtccacgaacgactccagctcggtgtactccttgttgttggtgaccttcttggcgtgccagaaatacgactcctcgttcacgttcatctcgtagaacgccagcaggaactcgtagtacacgctgtactcgcggcgatatcgcgcctcgaactcatgcagcgcgatcttctcctcgacgtcaccggccaggacgctgttgatcgaccgggccgccaggaggccgctgtaggtggccaggtgcaccccggaggagaacaccgggtccacgaagcacgcggcatcgcccaccaggatcatcccgggccgccagaacttcgtctggtggtaggagtagtccttgcggacccgcagctgcccgtacttgccggtcgtcacccggcgcgccggcgcgaggtactccgagatcagcgggcactcggcgatcagcgcggccagcgccttctcccgatcgccctggatcttctccgccatctcccggcgcaccaccgcgcccacgctggtcagcgtgtcgctcagcgggatgtaccagaaccagccgctgtcgaaggccacgctcaggatgttgcccgagtacggctccgccagccgcttgccgccctcgaagtaaccgaacagcgccaggctgcggaagaactccgaatagttccgcgtgccaccgacgctggaatacaaccggctcttgttgcccgacgcgtcgatcacgaaacgcgcggacaccgcgtgctcgccgccgtcaggatcgacgtaacgcaggccggtgacccgatcgccgtcctcgatcacctcggtgaccgagcatccctcacgcaccaccacgcccttgcgtctggcgttgccgagcaggatctcgtcgaagcgtgcccgctccacctggtaggcgaaagtcgtcggacccgtgatccgcggagagacggagaaggagaacgtccacggctccggccgcgccccccaccggaaggtgcccccgcgcttcacgggaaaccccgccgccgcgagctcgtccgtcaccccgagcatccggcacaccccgtgcacggtcgagggcagcaacgactcgccgatctgataccgcggaaagacttccttctccaccagcagcacccgatgaccctgcatggccaccagtgtcgccacggtcgaaccgccagggccgccgccggcgaccaccacatcgaactcttccacggacttctcctttttcgttgtggtcatgcgaagtcgcccggcatttcggccgcaggccgctaccaggtgaccggcaactcgtccgggcaatcgatgaacgcgttacggaacttcacttcctcggcggacaccgccaggcgcagcccgggaaaccggcgccacaaactctgatacgccatgcgcagcagcgtcctggctatcgccgcgcctatgcagtaatggatgccgtgcccgaacccgacgtgcgagccgcagtcacggcgcacgtcgaggacgttggcattcggcgtcagcgcctcgtcgcgattcgccatcagaatcgagcacaggacgtaatccccggccttgatcagctggccgtcgacgaccacgtcccggacggcgagccgtggattcggctgctgcacgggcgacaggaaccgcagcagctcaccgaccacccggtcggcctcctcgcgtccggcgaagagagactgtcgctggtcgggatgatccagcagcgcgagaaccccgaagccgatcgaccccgcgacggtttcgacaccacccaggatcagcgccgtgagtacgcccttcagctcctcgtccgtgacatcgtctccgtgctcccgcaccagcatcccgatgaacccctcgtcagggtccttccgctgccggatgatgaggccgttcagataccggttgaacgccgcgctgtcggccgcccgggccttgaacccgcggctgagatcgacgttctgcctgacacgccggatgaactcgatccgatcatcacgcgggatgccgagcagctcacacagcactcctccgccgaccggatcggcgaacagcgcctggacgtccgcgggcggccccgcggcctccagctcgtcgatgcggtcatcgatgaggtcctgcatggcgggctccagccggcggatccggcgggccgtgaactccggggtcagcatcccgcgcagccgcgtgtgctcgggcggatcatagaccgacagctgaccgaccagattcggcggtatgggctctccggcgatcgatggcgccgaactccaccgggggcgcgtcgtgaagttctcgtgatcgccgagtattctgcgcacgacgtcgtatcccaaagcctgccagacatagtcgacacgcagctgagtggccgcgtcaccgcctatccggaccagtgggccatgcgccctgagcgcgaacatgtcctcatgcggatcacagtgcgtccgcatcatgtagttcgccgtcggctgcaggaccggcgcgcccgcatcgatatcgtcatccatacccgggtcgaaactccattcgctgtcgatccgcacgctcggctgatccgatcctggtcgcgggcggaagatatttccagcgtcgtcaaatggacgatgggaacgggaattccgcgatcaccaggcgaccatcagactggtcaatccatacgcgggagtggtcaaccggaacgatggctcccgatcgggatccgcgagcctcagcgtgggaaaacgccgccacagggcggtgtagaccgtgcgcagttcgaggcgggccagagcggctcccaggcaatgatggacgccgtgcccgaacgcgacatgggggacgggctcgcgccggacatcgaggcggccggcatccggcagcagggcagggtcacggttggccatgggcagagagcaggagacggtctctccctccttgatcacctggcccccgatggtgacgtcctccatggcgacccggggcgtcggcgcataggggacggtcaggtagcggatcagctcgtcgaccgcccgatccgccgactggtcgtcgccctgcaacgcggcgatctgctcggggtgtctgagcagggccagcacgccgagcccgatcatgccggagatgttgtcgtcgccggccagcatcacctgaacgcagaagccccgcagctcctcgtccgtggccgtgtcaccgtactcggcgaggacggctccgagcagcccctcgccgggatccttccgctccctggcgatcatggccagcaggtagcgggagaacgccgcgccggcggccgcccgcctcttctggctgcgcgaggcgtcgagatggccgtgacacagctgcatgaacatggcgcggtcgtcccgtggcaccccgatcagctcgcacagcacggcccctggcacctcgtcggcgacgagttcgaccagatccgcaggggggcccgcccgttccagggcgtcgagccgttcggtcacgatctgttcgatgtacggcttcagccgccggatccggcgcagggtgaatcccggggtcagcttctggcggagccgcgtgtgctcaggcgggtcgtagtccatcaggttcccgaccagctcacgcggccggaagttgcctcttccgccgatctcgtcccgttcgttccagcggcgccgggtgctgaaccgccggtgatcgccgagcacctgccgcacgacggtgtaccccgtggccagccaggtggtctccgcgtccgctcctgatccgatggtgatcctcgtcagcgttccggcggcgcgcagttcgtccgccggatccaggtcctgccgccgagtatggaggggccgcgcgccgtcaccactcaacgggaagctcctccacggcgaagggggccggcttccccggtttgaaccgcaggtcctctgccgggaccgccagccgcagcgacgggaaccggcggaccaacgccggcaacgccacttgcgcctggagcctggccagcggtgccccgaggcagaagtggacgccgtgcccgaacgcgaggtgttcggggttgccccgggtgaggtcgaagcggtcctcgcgcgcacgattgcccgccaggatcgagcaggtcaggacatcaccggcgtggatgtcccgtccggccaggcgcgtgtcgaccagtgcggtccggggagagggggtctcgacgatcgacgcgtagcggaacacctcctcggccgcgctgtccgcgagctccggacgctcgcgcagcagcgccatctggtcggggtgggtcacgaggaggagcaccgcgatcgccagctgcgaggccatctgctcgaccgccccgatcatgatcccctcgacgagccccgccagctcctcgtcggtgacctcgcctccgtgctcgcgcacgatgccgccgatcatcccggtgccgggatcgaggcgttcgcgagccgccagtttcctggcgtagtcgacgatgcccaggcccgacacgttccgctgcctggggactcggctctcccggctgtcccggaacatcctcgacagctcggcctggtcgtcacgggggacgccgaggaagtcgcaggcgatcagcgccgggatgggccaggcggcgttcctgacgaagtcgaccggcgaccccatgctctccagatcggccaagcagtcctcgacggtctcttcgacgacaggccgcaggctctcgatccgccgggcggtgttcgccctggtcacggtcctgcgcaaccgggtgtgatcgggcgcgtcgtacgactgcaggattcccggcagccaggcgcgctccgcctcgtcctcgaccgggcgcatcgagctgaaccggttggcgtcggcgagaatctctctgatctcgccatatccggtgacgagccactgcttgtggccgtccagccccggctcggtgtcgtactcgtgcagcggcccgtcctcttgcaggtcgaagagcgccggcacgggatcgagcctcagccgctgatgcggcagcggaaccaccatgattctcctcagcttccggcgttaccagtcgagcagcagtgctttcacgtcgaacggcggcggccccaatctgatctcttgttcgggctcggccagccggagtgcggggaagcggcgtgccagagccgggatcgccgaccggaagatgagctcggccaggggtctgccaagacagtgatggatgccgtgcccgaacgcgacattcggcgctctgtcgcgggtgaggtcgaactgatcgtccgggccggggaagtgccgccggttcgctcccagcaacgagcacgtgacggtgtcgccggccttgacgatgcggtcaccgatgcgcacgtcctccagcgcgatccgcggagtgagcttctcgtcgatggtgagatagcgcaccatctcctccagccagtcaggcacgacgtccggcttgtcccgaagcagcgcgaactgctcggggacctcggccatcagccacgcgcccgccgcgaggaaacgagccacctggtcaccgcccgcgcccatgacgaacgcggccagccccgtcagctccgcgtcggtgatctcgtcaccgtgttcgcgcaccacgacgctgagcatgtcgtcgccgggatcacggcgcgtgcgagccacgacctggcccatgtaggtcatgaacttgttcccggcggctccgcgtctgctggccgcccgctgggaccggctggcgtgcaggctgcgtgacagctccgcctgatcatcacgtggaatgccgaggaagtcgcaggtcgccgtcgtcgcgatggaccacccgaaatgcgggacgaagtccagcgggccaccgatggactcgatggcgtccaggcagtcctcgacgacctgatcgacctgcgggcggaaccgctccatccgccggacggtgaacgcgggcgagaccacccggcgcagtcgcgtgtgctccggcgggtcgtactgggtgatgaagccgggaaagacgattccggccgcggtcccgccgtagagcaacctggagctgaacttgtccgagcccagcacctgccggacctcgtcgaacccggtggcgagccacgcggtccgccctccagggccttcctcggcgcccagctcggtcatcgggccctcggccatgaaggaccgcagctgtggcaccggatcgaaccggtccctccagtgcagctccccgggcaggacgacgttgagctcctcgaacacttccacgtcacaggtccttccgcctcaacggtggtctcaggccggtcggacgggcgctgggcccgtccaaccgtccacatcaagcggctggacaccctcgctcaggcgccggcttccgcgatgagactcttcggccggatgtccgtccagttctcctcgacgtaggcgaggcattcctggcgggtggccacgccgtggacgcgggtccagccaggcggcacctccgcgaacgagggccagagcgagtgctgtccttcgtcgttgacgagcacgaggaaggagccgtcttcgttctcgaacgggttggtcatcgctgtgtcctttcaccgtccggccggggccggagtttctcggcgacgacggccccgatccgggccagcgccgcgggctgcagcatctgcatgtggtcgatctcgatctcgtgaggctcgacggttccggtggtgaggggtcgccagctggcgatggcgtccgcgacgggcagatgggaggggcggttcactgtggcgacgaagagcaggatgtcgcagccgaagctgcgggaggtgtgcagcggcccgacccgggcgaggtgctccatgacctcgtccaggcgcttcctggcgccggcctccgtggccaccgcggcggcgagctgcgcctgctgctcctgctgcctgtcgaagtccgcggcctcctgatcggccgcgtcgccgcgcgggcgccggagcctgcccacgtcggtgggataggcgtcgagcagggcgagcaggccgacctgctccccttgctcctccagcaccctggccatctcctgcgcgatccgcccgcccagtgaccacccgaggaggtggtacggcccggtcggctgcacggcgcggatctgctcgacgtaatcggccgccatctcctcgacgccggccgccagcggctccgtacgagccaggccgcgcgcctgcacgccgtagaccggctggttggggggcaggctccgcagcaggggcgcgtagttccagctcagccctccgctcgcatggacgcagtacagcggcggacggttaccgccggctcgcagcggcagcagcacctcgaagtcgcccgtgccggtcttcaccgagccgctcgatccccgccgctcgccgacgacgaccagggtgcccccggctgtccagcgggcgagcaggcccgtgggcagcatgcgccgccccgtggccccgaaggggcaggccagggtccggtcgcccggctccgcgtccaccgccgcgccggtgaggtagagctcgccgacggcgacggcggggcgcagccggtcgtcgagcacgagcgcgccgagcggaccgccctcggccaggtcggcggccaccgggggcacggcctcggaccacttcgccggcgcctcggccggacgttcgcgttcggcgtcgtccagcaggacgtccaggtcgctgacgcgccgctggggatcctccgccacctgctccaggaagctgaccagccgccgtgccagcgactcggccgtggcctgatcgaagaggtcggcggcatagtggagggtgccctcgatgccgtcctcgtcgcggcgctcggtgagcctgaacgccagatccagctcgatggcctccggcccgacaggttcgacgctggtgcgcagggcgggcagctccgtcgcgtcccacgcgccgaggtcctcctcgtgcacctccaaccccacctggaacacgggatggcgggagagcgagaccggcaggtcgagcagctcgacgatcctggcgaagggcacgtccaggtgctggcgcgcggaccggatcgcctcctgcgcccgggtgacgacctccaggaaggtggggtcgcccgagaggtccgtgcgcagggcgagcggccgggcgaagggcccgatcatcggctccaggtcgatgaggtcgtcgtcccgtggcagcttcgtgccgatcaccaggtcgtggcccgccccgagcctggtcagcagcatggcgagcgcggcatgcacgatctggaacgggtgcgcgccgatcgggtccaccgcctccaccagcctggcgtgcgggccggcgtccagtcgcaacgacaccgtgccggcccgccgcgacgcgacggccgaccgggggcggtcgaacgggagcaccgtctccccgtggatgcccgccagattgtcgcgccagaacaccagctgctcgttgatcaggccgtccgcatcgcgctcgccttcgagcaggcgccgctcccagatcgcgtagtcggcgaactgcagtgtcaggggcgcccgctccggtgcccggccggcacgccgcgcgccgtacgccgccgacaggtcccggaggaacacatccagcgactcgtcatcggcgaggatccggtgcaccatcaggtgcaggacgtgttccccgtcggagagccggaacaggtcaccgcgccacggcacctcccgggtgaggtcgaagaccgactcccgcagctcggtgagcagcccgggcaggctctcctcggtggcgggaaccggcgtcaggtcaaccggcgaggcgtcgtgtacgtgctggtgaacgctctgcgcgtggccggggaaggtcgtccggagaatctcgtgccgcgccgcgacgtcgccgagcgccgcctccagcgcgggcacgtccagccggccgcgcaagcgcagcgcgaccgagacgtgcaggccggcggcctccccgggactcgccagcagccaggcgctgagctgctgggcggtgagcggtacccggcccggccgttcggcgggctccagcgcggggcgtgacttcgcggccagcgcccgggcgacacccgcgggggtggccgcggagaacagctgccggatgggcaggtccgcaccgagctcctcacggatccgcgcgatgagccgcatggccagtgccgaactgccgccgagatcatggaaggcgtcgtcgacgcccacccggtcgacgccgaggatctcggcgaagagcgcgcacagcaccttctcggtctcgctctcctgtgccctgtccggcgcacgtcccacgagatcgggggcgggcagagccaggcgatccaccttgccgttgggcgtgacaggcagcgcgggcagggcgacgaccgccaggggaaccatgtacgcgggcaggaccagggccatctcccgccggatctccgccggccccgcatcggttccgtcggagatgaagtagccgaccaggcgcttctcgcccggctggtcctcccgcgccacgacgaccgcctcgaccacgccgcgctgggccgccagcacggcctccacctcgccgagctccacccggtagccgcggatcttcacctggtcgtcggtgcggccgaggaacaccacctcgccgtcgcggttccagcgcgccaggtcgccggtgcggtacatgcgctcaccaggagaagggtccacggaggccggaacggccacgaaccgctctgccgtcaggcccggcccgccgagatacccgcgggccagcccggtgcccgcgatgtacagctcgcccgccacccccggcgcgaccgggcgcaggaaggcgtccaggatgtagatcctgcggttggtcatgggacggccgatcggcagctcccgcccgacctcctcgccgggctcgatcggcttccacgtcgcgcacagggtggtctcggtcggtccgtacgtgttgcgcacccgcaggccgggcacggcccgccgcaggtgctccacggactgcgccggaaccacgtcaccaccggtcccgacctcgaccaggcccgcgaaacactccggggacgactccgcgagggcccggaaggtaccggcggtgagatggacgaaggtcacgccccgttcgacggcctgtctcatcccgagcgcgtccagcactcccggctcggtgagcacgacccggccacccatggcgagcggcacccacatcgcgtagagcgaagggtcgaagacgtgcgtcgcgtgcatcagcacggcgtcgcccgggccgatccgccagccctcgtcgcccgccaggccggccacggccccatgggggacgcccacccccttcggcaggccggtggagccggaggtgtacatcacgtacgccaggtcgtcggcgctcagccggatctgcggcgccgtggcggcacccgcgtcgatggccgcgcgggtctccggggcgtcgatgacgatcgcgtccgccggcgccacttccctggtggcccgggtgcacaggacggccgagacaccggagtcggcgagcacgaactcgatccgctcggccgggtgctcgacgtccaccgggacgtacgcggcgcccgccttccagctcgccaggaacgcgatcagcaggtcaggcgacctgtccatgaccacgccgacgcggtcgccacggccgatgccacgagcggcgaggtggcgggccagccggttcgccgcctggtcgacctcggcgtaggtcaggtccgcgccgccggcatcggtgatcgccaccgcgtccggcgccgtggccacccgccgtccgaagagatcgagcacggactgccccggcgtggggcccgccgtcgcgttccagtcctccaccaccagggcgcgctcggcttcgctcagcagcgtcagccggccgacgaggacgtcgggctcggcgaccagccgctcaagcacgcgagccagcgcgcccacgaccgattcggcggccgcctcgtcgaagaggccgcggtcgtagtcgaggatgagcggcatctgcgcgccgggcccggtgatcagggtgaacgggtagtgcgaggagttgcgtccgcgccgcaccggtctcaggtccaggccgccgtcctcttcggcccggccgagtccctggcgcgggaagttctcgtagatgacgagcgtgtcgaagaccgccccgggcccgacggcggcctgcatctcctgcaggccgagatgctggtgcgccatgagtgccgactggctgcgctgcagctcggcgagcagctcgacgacccgccggccgccttcgagccggacccgtaccggcagggtgccgagcagctgcccgaccatcgactccacaccggccagctcggcgggccgccccgaggcggtcgcgccgaacaccacgtcggtgcggccggcgagctgcgccaggaccatggcccacgcaccctggacgacggtgttcaaggtgaggccgtggccgcgcgccagccgcgccagcccgtccgtcagctccgcggagagctcgatcaccgcggtgcccatgtccggcacgcgggccggatcggccggggcgaccagcgtgggcgtgtccagtccggcgagctcctgccgccaggccgcccgggccgcctccttgtcctgccggcccagccaggcgaggtagtcccggtaggacacggcggcaggcagcccggacgcgtcgccgcccgccgcgtagatcgcggccagctcgcggtgcaggatcggcatcgaccagccgtccagcaggacatggtgcagggtgtgcacgagccggtggctggccgggccgagccggatgaggtgcagcttcatcagcggtgccgcatcgagggggagccgctcggccagctcgtccgccgccagccggtccacctcgctgtcgaggaggtcgtccggcagcccgtggagatccgtttcacgccaggggatctccgcctcccgcgcgatgacctgcaccatctgcgcgccgctgacatagcggaaataggcccgcagcgcggcatgccggtccacgagcgcctgccacgacgccctcagccgcccggcgtccagcgggccgtcgatgccgtacacggtctgcaccgtgtaggtgtcgggcccgtcgtcgtcgagggcggtgtgatagagcatgccctcctgcagcggcgagaggggccagacgtcttccacgctggagcgcggcttcgcacgagtgtcgtcaatggtcacgatctgctccttatggagtcatccgccggccggtccggcctcgagttggtccagctgatcaggcgaaagatccacgagtgtgccggccgggggctccggttccccggtgccctcgccgtcggccgggagctccttgacgaccgccgccagccgttccgccgtcttctcgtcgaacacctgccacggggtgaggtccagccccttccggcgggccagggccgacagctgcatcgaggtgatcgagtcgccgccgagctcgaagaagctgtcgccggcgcccacctcctccaggccgagcacctccgcgaacagctcgcacaacttcgcctccatggccgagcgcgggtcgcgtcctgacgacgacctcgcgaaatcgggggcccgcagcgcccgatgatcgaccttgccgttcggggtcaggggcatcgtgtccagcgggacgaacgccgccggcctcatgtgctccggcaggcgtccggccgcgctctcgcggagggcggagatcaacgcgccgtcctgcccggcctcggagggagcgccggccacctgctcggccgcggggacgacatacgccaccaggtacttctggcccggaccgtcctcgcgggcgacgaccgccacctgggcgacccccggatggtccgccagcacggcctcgatctcacccggctcgatccgatagccgcgcaccttgacctgcgcgtcggcccggccggtgaagaccagctcaccgtcccgggtccagcgggcccggttgccggtgcggtacatgcgctcaccaggacgggcagggctcaccgaggccggcaccgcgacgaaccgctccgctgtcaggcccggacggccgaggtagccgcgggcgagcccggcccccgccacatagagctcgccggtcacacctggcggcacgggctgcaggaaggcgtcgagcacataggctcgcaggccggtgatcggccggccgatgggcaccacgtcgcgtcccggagacagcggggagctcatcgtcgcgcagacggtcgtctcggtgggcccgtaggcgttgatcatccggcggcccggcgaccagcggtccaccagcgcgggcgggcaggcctcgccggccacgaccagggtctccaggctgtccggcaggtcgtcctcgacggccggcacgctcggcggcacggtcacgtgggtgatgccccaccggcgtaccgcgtcgcccagcgacacccggggcggcatgctctccgcgtcggccagcaccacggtcccgcccgacaacagggccatgcacagctcggagacggcggcgtcgaagccgagagcggcgaactgcaggatccgcgaggcggacgtgacgccgaagcgctcgatctgcgcgctcgccagattgccgagcccggcatgggggacgaggactcccttgggcacgcccgtcgaccccgaggtgtagatcacatacgccccgtcacccgcctccacccggggcagcgcagtgcgcggatcggcggcgagcggcgcgtccagcgccaccaccgcgcccgcgaactcctccgggacggcctgcctggtctcgctcgtgcacagcagcacctccggcgcggaatccgccaggatgaagctgatgcgctcgcgcggataatcgggatccatcgggacgaacaccccgcccgccgaggacaccccgagcagtgccaccaccagctcggccgagcgtcccacgagcacgcccacccgcgtctcacggcgcacgcccagccccaccagcagccgcgccagctcctccgcctcgtccagcagtccgctgtacgacaggctccgggccgcgtccaccaccgccaccgcatccggcgagcgctccacctgccggcggaacagcatcggcaccggctccgcggcgggcggcacgccggtcctgttccactcctccaccaccaggcggcgctgctcaggaccgatcaggcccacgcgcccgaccggcacgcgcggctcggccaccacctgctccagtgcccggaggatcgacgcgagcatctcctcggcctcggcccgatcgaccacgtccggccggtagatgaactcaccgtggacgcggcccgccacggacgcgcgcatggacagcggatagtgccctgtgtcgttcgggatgcccgcgggccgcatgacgagcgcgtcggggccttcgggccgaggcggcgggggcgggtagttctcgaacaccacgatcgtgtcgaacgccgcgcccggaccggcgagctggttgatctcgctcagccccacgtgctggtgcggcatgcacgcgacctgccgttcctgaaggtctgtcagcatgtcgaggaacggctccgcaccggccaggcgagcccggaccggcaacatgttcatgaacaggccgacggcggactccacaccggggatctcgggcgggcgcccggccaccgcggcgccgaagaccacgtcgtcgcgtccggtcagccgggccaggtgcagtgcccagatcccctggaagagcgtgttcgccgtcacgccgtgacgaccggtgaactccaccacgcgccggctcagcgcctcgtcgagttcgaacccgacacgttcgggctccaggggagtggtgatcgtctccggcggcacgacgtgggtcgcctcgtcgagccccgccagctcggcccgccacgcctctcgggcggccgccttgtcctggcgggcgatccaggcgagatagtcgcggtacgacgtcgcggccggaagggcccggccgtcaccgccggactcgtacacggtcagcacgtcctcggtgatcagcggcagggaccagccgtcggccacgatgtgatgcgaggtcaggaccagccggtgccggcgttcgccgaggcgcaccaggtgcagccgcagctgcggcgcccgggtcaggtcgaaccgctcggtgtgcagctgctccgcgaggcggtcgaactccgccagcgcctcgtcctcgggcagccgggacagatcggtctcctgccagtccagcggcacctcgcgagcgatcgcctgcacggccgcgccggacccgagctggtggaagctcgcccgcagggcggggtgccggtcgagcagagcctgccaggaggcgcggaaccgggcgacgtccaacggaccgtcgagggcgagcttgcgcatccccgcgtagacgtccgggccgcgctcgtcggcggcgtggaacagcaggccctcctggagcggagacagcggccagatgtcgagcagggtcggtacggcggcctcgacctccgccacgtcctgctgcgtcagcgagatgagcgggaagtccgacggcgtgtgcccgccggcgccgccgccaccgacgtgtgccgcaaggccggtcagcatggccagccaggcctgcgcgagcgactccgcctcggcctcgccgagcagccgccccgcccaggtcacggtcaggctcagctcaggtcctgccgcaccgtccagcacggccgcgtcgatctccacggcgtgccgcaacgccgtgtcctgctccgccgtgccgccgatggtgcccagcagctgccagggctccggggcgcccgcggaccgggacgggaagcggccgaggtagttgaacccgatctccggcttcggcgccgccgcgagagcctgccccgtcccggcgttgagatagcgcaggatcccgtagccgagcccgccgtcgggcacggcccgcacgttctccttgacctgcttcagcaggtgaccggccgcaccgccacccgcgatcacttcggccggatcgatccctgtcacatccagccggagcggatgcacgtcggtgaaccagccgaccgtccgcgacaggtccagctcgtcgatgggccggcggccgtgaccttcgacgtccaccacgaccgcggtgccgccgcgccagtgggccaccgcgcctgccagcgtcgccagcaacacctcgtggacaccgcagtggaaggcggaggtggcctgctccacgagcacgcccgcccggtcatgcggcagcgtccacgatgtgcgtcccgcggtcgagacggtgtcgcgcgccgggtcgagctcgcccagccgcgatcgcgccccgtcgaggatctccgtccacgtctccagctccgtggcccgtgtcaccgcctgatcggccagcgttcgcgcccagcgccggaacgagacgtcgacggggtcgagcaccggccgccggccggcggccacggcttcgcaggccacctgcaggtccggcagcaggattcgccacgacacgacgtccaccacgagatggtgcgccgccacgacgagccgtcccacccgccctggccccgcgtccacccagaccgcccggatcatcacgccggcgtggggatccagccgtgcggccgcgtcgcgggcgcagcgatccgcgatctcatccacgtcaccggtgccggcctcgacccgttcgaccagcgtcgccgcgtccaccgctccgcggccggccacgaccagccggggctgcgcagccccggtgcggacgatccggctgcgcagcatgtcatgcgcgtcgatgaccgcgcccaatccggccgccagcacgtccaccgacaggtcgtcgggcgcaccggcggtcacccactgggacagggcgccccgggtcatcgcgtcgggatcgcgttcgagcagtgcccggatcaccggcgtcgacatcacctcaccgacgccgtcgtcgaggctcgcccgcgtcgcaccgccgcgttcggcgaccatcgcgatcccggcgggcgtcttgcgctcgaagacgtccttcgcgccgaagacgagctcctcacgtcgcgcgcgggcggccaaccgcatggagaggatcgagtcgccgcccagctcgaagaagctgtcctcggctccggctcgcgccacgcccaggacctcggcgaacagttcgcacaacacccgctcggcctcggtacgcggctcccggccggccgccttcccggtgaactcggggacgggcagcgcggcacggtcgatcttcccgttgggcgtcagcgggacgccgtccagcagcaccaccgccgccggcaccatgaactccggcagccgtcccgcgaggtgctcgcgtaccgcgtcgggatccaggcccgagccctcctccgcggtcacgtaggcgatgagtctcttctcgccgggacggtcctcccgcgccacgacgaccgcctgcgcgacgtgcggaacctcggccagcgccgcctcgatctcccctggctcgacgcggtagccccggatcttcacctgggagtcggcccgcccggcgaacagcagctcgcctcggtccgtccagcgcgccaggtcgccggtgcggtacatccgctcaccggaggccgcggggttcaccgaagcgggcaccgcgatgaaccgctccgaggtcgccgcgggggcgcccaggtaaccgtgtgccaggcccgcgccggcgaggtagagctcaccggtgacgttcggcgccaccggctggaggaaggcatccaggacgtacacctgccggccggccagcggacggccgatgggcaaggtgtcgcccgtttccgtgtgcggctcgatgaggtgccaggtggcgcagagcgtgacctcggtggggccgtacaactcccggacccggacctccgggcatgcccggcgcacccgtgcgacggactcgagcggcaccacgtccccgccggtgaggacctcgcgcagcccgctgaaggagtccggcgactcctccgccagcacccggaaggtccccgccgtcagatggacggtggtcgcgccccgtgcgatcacgtcccgcagccgctgcgcgtcgatcgcgcccggttccgcgaccatcacgcaggctccgctgaccagcggcacccagatctcgagcagcgacgcgtcgaacgcgtgcgacgcgtgcatcaacacgcggtcgccggcgccctgcgaccagcccgggtcgccggccagagccgcggcgctcccatgcggcaccgcgacgcccttcggcaccccggtcgatccggacgtgtacatcacgtaggccacgtcatgcgctccgaccgcgagcggtggcgcctcgtgccgctccgcctcggtcgcgggtgcgtccatgaccaccggctcgatcccgtccgggaccgcgtgccgggtcgctcccgcgcacaccgccaccgacgcgccggcgtcggccagcatccgctcgatgcgctccgccgggtagtccacgttcaccgggacttgcgcggcccccgccttccagaccgcgagcagggcgacgatcaggtccgcgccgcgttccatcagcacgccgacgcggtcgccgcgccgcacgcccctcctggccaggtgccccgccagccggtccgattcccggtcgaggccggcgtaggacagggtgcgtccgtcgccgatgaccgccgtcgcgtccggcgccgcgtcggcctggcgccggaacagctccggcaccgatgaaccgccggccgccgcaccggtcgagttccagcgctccgtcacggagccgcgggtggatcggctggtcacggccaggcggccgacgggaagcgagggctccgccaccatccgggccaggacccgcacgacctgcccggtgatctcggcggccaggtccccgccgatccagtcgggccggtagtccagctggatctgcaggcgcgcgcccgggatgacgctcacggacagcggataggtggtgccggtccgcgtgcggatcgagctgatcgccacgccaccgtcgtcgagaccgtcggcgtccagcgggtagttgacgatcatcaggatcgtgtcgaagatcgagcccggccccgccgccttctggatctccggcagccccaggtgctgatgctccgtcaaggacgactggcgccgctgcaggtcctggagcaggtccagcaccggaacagccccgtcgaggcgcacccggaccggaacggtgttgatgaacatccccaccatccgctcgacatccggcaacgcgtccgccggacgcccggacacgaccgtgccgaacaccacatccgtccgtctcgccagccgcgccagcaccagggcccaggcgccctgcacgaccgtgctcaacgtcagcccatgaccacgggcgaagccggtgagggcgcgggtcgcctcctcggacagccattcggcatgcccgtccggcatcaccggcgccttgcccgcgtcgaggcccaccacggtcggttcgtccagcccggcgagctcggcccgccacgccgatcgtgccgcgtcctcgtcctgacggctcagccacgccacgtagtcccggtaggagggcggcgccggcgagacccgtccgtcggcgtaggcggtcagcatctcgcccagcaggatcggcgtggaccagccgtccacgagcacatggtgcgacgtcaccacgagccggtgccgcgccgcaccgagacggatcagcagcaaccgcagcagcggcgccctgctgacgtcgaaccgctccgcctgatccgccgcgagcaggcgttccacctccgcgtccggctcatcgagccgcgacaggtccgcctcacgccacaggacctcggcctcgcccacgacgacctgcaccgtctcgccggatcccagctggtggaagcccgtccggagcgtctcgtgccggtcgatcaccgactgccacgccgcgtgcagccgttgcgcgtcgagcggcccgtcgaggtccaggatccgctgggtctggtagacgtcgacgccgtcctcgtcgaaggctctctcgaagaggatgccctcctggagcggcgacagtggccagacgtccgtcaggccgggcgccacggcctccagttcgtccacgtcccgctgccgcacctcgaccagctcgaagtcggacggtgtgtgtccgcccgcgccgggagtgtcggcgagagcggcgaggccggccagcgtgtccagccacgcctcgccgagccgctccaccgcggcagggtcgaggtccctgccgtcgatggcgagtctcagccgggggccggccggcgtgtcctgaacgtccgcgccgacctccagggcgtgggactggacgaggtccggcccggccgcctgtccgccgagagccccttcgcacacctgccacgcggtgtcctcggaggcgacgccggaccgtccgagatagttgaatccgatctgggccgacggcagctccgccagccgggcgccggtttcggggttgaggtagcgcaacagcccgtagccgagcccgtcgcccggcaccgctcgcgcctgttccttcacggccttcagcaactccccggccgccgcagctcctggaccgacaccggagacatcgaggcggaccggatgaacgctggtgaaccagcccacggtacgcagcagatcctctccgtcggcggcatggcggccgtggccttccacgtccaccaggatcccggcgtcagcaccgcgccaccgcgccaccgcacccgccaggcccgccagcaggacgtcctgaaccccgcagtggaaggctgccggcacgcgcgccaccaggttgcgcgcttgggcatcggacagtgtccgcgaccacgacgccgactgcccggggtgccgctccagcggcaggtcgccgccttcgagcacgccggcccaatggccggcctcggccacggtgctctcgctgagcgcctgcccggccagccgccgcgcccattgccggtacgacgtcaccgcgggttcgaggacgggggttccgccggagaccgcctcgtcgtaggccgcccgcagatccgacagcaggatcgcccacgagaccgcgtcgacgaccaggtgatgcaccaccaaggccaaccggcccggctcggcgtcgcccgcgtcgacccacacggcccggaccatgatcccttcggacgggtccagcgtgcccgccgccgtcctggcctcgcgctcggcgcgctcagcgaggttcccgttcccggccgccacccgcgtcaccaggccggccgcgtccacggcacccggctcggccaccatcagccgtccgtcgggctccacccgcgtccgtagcagatcgtgcacatccaggacggcctgcagggcggtcaccagcgcgtccggggcgaagccggccggggtgacgacgacccgcgcctgcgcgaaaccggggcgcaccgcgtcatcgccgagggcacgcatcaccggcgtcctcgggatctcgcccacgcccggctccactgaggaggctcgcctccccggggcctgttgagccagcgccgccagccgctcgggcgtgcggtgctcgaacacctgtcggggggtcagcgggataccctggcgccgcgcgcgggcggcgacctgcatcgacgagatcgagtccccgcccagctcgaagaagctgtctgcgaccccgacccgccccgcccccaggacctcggcgaacactccgcacaggatccgctcggcgtcggtggccggctcccggtccaccgccccggcagcgaagtccggctcgggcagggcccggcggtccacctttccgttgccggtcagcggcaacgcgtccagcaccagcaccgcggccggaaccatgaactcgggcagcgtcgcggcgagctgctcgcgtatccgcaccgggtcgaggtccccccctgtttcggcgaccacgtaaccgatcaggcgctcttcccgcgcggacaccacggcctgaccgacacctggaaggccggcgaggaccgcctcgatctcgccgggctccacccggtacccgcggatcttcacctggtcgtcggcacgcccggcgaacgccagctcaccctgatccgtccagcgcgccaggtctccggtccggtacatccgcccaccgggcacgaacggctcggcgacgaaccgctcggccgtcaacgccgggcggcccagatagccctgcgccaccccggccccggcgacgtacagctcgcccgtcacccccgggggcacgggccgcaggaacgcgtcgaggacatagacccgccgccccgcgagcggacgcccgatcggcagcaccggccccgtcggctcgcccggctgcagcagccaccatgtcgcacacagcgtggcctccgtcgggccgtagagatgccgcacgcggacgtccgggcacgcccgccgcacccgttccaccgccgcgagcggcaccgcgtccccaccggtcagcacctcgcgcagcccggcgaccgactccggtgactcctcggccagcacccggaaggtccccgccgtcaggtgagcgcaggtgacaccgccggccacgtacccggccagggcctcgccgtccaccgcgcccggctcggcgagcacgacccgggcgcccgacagcagcggcacccacagctcgaacagcgagatgtcgaacgcgtgcgaggcgtgcatcagcacggcgtcctcgggccccagcccccatcccggctcgccggccagcgccgcgacgttgccgtgcgagaccgcgacgcccttcggcctgcccgtcgatcccgacgtgtacatcacgtacgcgaggtcgtccgcgtgcgcacccgcggagagacgggcgtgctccgccaccgcccgcagcgtgtccgggtcgtccaggacgatcggatcgagcccgccggccggcacggcggcctggcacgctcgctcggtcaccactgccgccggctccgcgtccgcgagcatgaactgcacgcgctccgccgggtaggcgggatccaccggcacgaacgccgctcccgccttccacaccgccagcagcgtcgcgatcaggccgggtgaccggcccatcaccacggccacccgatccccgcgccggacgccccggccgctcaggtagccggcgagcctctccgcgtgctcggccagctcaccgaacgagaccgcccgcttcccctcgacgaccgccacccggtcacggccgcgctccacctggcggtcgaagagatccggagccagctcgcccggcgccacgcggggtgccgcactccatgcgttcaccaccagcgcacgctcggccgcactcgtcacgtcgacctcggccaccgtgaggtcgcccgcgcccgccagctgccgcagaatcccggtgaatcgctccaggatggcgagcgcggcttcccggtcgaagaggtccgtcacatggtcgagattgagcagcatcgactcgccggggacggcgaccagcgtcagcggataatgggcggcttcccggccctggtctattcgaatatcgaaggctgccgcggcatccgatgggcgaagctcacggggaaagttctggaaaacgagcaaggtgtcgaagacggcgccggcgccggccgtcctctgaatatccgccaatcccatgtactggtgggggatgagcgccgactgccgcttctgcaaatccgccaggaattcgatgaccggcgtcgaaccgctcagccgcacgcgtacggggacggtgttgaggaacaaccccaccatcccctcgacgccgggcagatccggcgggcgtgccgagaccgccgcaccgaacaccacgtccgtccggcccgcgagctgggcgagcagcaacgcccacgcgccctgcaccactgtgttcagcgtcagcccatgggtgcgagccagctcgctcagggctcgcgtgaggtcctcgggcagctcgaccgtgatgttctccggcatggcgggcgcccggttcgcatcggcgggcgccaccagcgtcggctcctcgacaccccgcagctccgccgcccatgccgacagcgtgcgctccttgtcctgccggtccagccacaccaggtaatcccggtacgacggcaccgcgggcaggtccagcgggctcccgtcggccgcgtacagcatcgacagctcgtcgagcatgatgggcatcgaccagccatccatgatcgcgtggtggcaggtcatcaccaggcggtggtcgtcgccggcgagacggatcagggtcagccgcagcaacggcgccttcgcgaggtcgaacctccgcgtcctgtcctcctcggccaccgcgcgcacggcctcctcgggctcgctgaggtgggagaggtccaccacccgccacggcagctccacctgcctggcgatgagctgcaccgtctcacctgatttgcgctgccgaaaacaagcccggagagcggcgtgccgcgccaggagcgcctcccatgcggcacgcagtctgtccgcttccaccggaccgttcaggttcaggatccaatggcccacataaaggccgggccagtcgtcgtcataggtcgtgtggaagagcaacccttgctggagtggtgacagcggccagaaatcttcgatccgcgactgagccatggatgaatatctccctcaatcagcaaagcggcccgagagggaatcatccattgatgggtctgacccggacaatctgtccatccgtgactgccgtcaccgatccgggtggggtcgaaggaggccgccgacgcggaacgtggcggcttgcgggcgagcaacatggctacggcgcgccatccacagctggatggcgcgccgtagccaggttcaccgctcgatcgagcgcggcctcactcgaaggaaagccccgcggccggcgtcacccggacggcccggcgtgccgggagaacactggccaggagccccgccagggcggcgacgaggacgacgacggcgagcagtggccaggggacctgcatggtggcgttgtcgagagcctgcttcacgaaggtctcgtaaccgacccaggcgaacccgatgccgatcacggtgccgagcacggcggccaccagggagagcagcacggcctcggcggccagcatccgccgcaactgcctgcgagtgagcccgagcgcgcgcagcagcgcgtgttcgcgaacacgctcgagaacggacaggcccagggtgttggcgatcccgacgagggcgatcgccacggagaagccgagcagcgcgacgatggcccaggtgaggatcatcagcggcgcgttctccgtctcacgggcctccagctggtcgttcacgttcgccccggccgcggccgccaggtcgcccagctcaccgacgagccgcgtcgagtcggcgtcggcggatgcgcggatccagacggcacgcggcgcggcggagtcggtgagccgggccagcgtctccggcgcgacgacggcctgcagcccccagccggtggcgagcgagacctgcagcacggcccgccggtcgccgaccgtgaccctgaccttgtcaccggcccgcaggcgcagctggcggaatgcggactcatcgagcctgagcacgcctggctccacccgggcgaacgacccgccgtcgtgggccacccgctgggcatccggcgcggtgaccaccgggatcggcttgtcgaggccggagaccgtggcgacggcgccgtccaccgcgatggcctgatccaccccggaagtgccacggaccttgtcgaggaagtcggcggagaacggcttgccggtcgagaccagcgcggcgtcgatggggtgctggccgtcgagtctctcgttcagcgcctcggaggtgatggcgacgccggtcaggacggcggtgatcagggtgataccgaccagcagtgaggcggcggtggtggcggtccggcgcgggttgcgcacggcgttcttcgtcgcgagccgcccgatggtgccgagccgcgtaccggtgatctccagcagacgggggatgagcaccggcccgaacaggagcacgccggtgaacaacgaaccgccgccggccagcatgagcaccgtgctgtgccaagccatcgccgacgcgagcaggacgagcccggcgatcaacatgaagacgccgagcaccagccgtgcccgccccgtggctgtacgcgggtcggtcgcggtgtcgggacgcagtgccgccagcgggctcacccgcaccacgcgccggatcggcagccaggccgcgaccagggtggccgtcagcccgatggcgagcccgcccagcagccacggcgcgggcggtgccggggcggcgatcggggtgatcggtgagagggtcttgatcagggcgatgagcccgtagccgagtccggcgccgaccagcacgccggccagcgacgacaggaggccgacgacggccgcctcccggcgtaccgaactcaccacctggcggcgggtcgcaccgacgcagcgcaacagggcgaagtcgcgcatgcgctgggccagcaggatggagaaggtgttcgcgatgaccaggatcgagacgaacacggcgatgccggcgaagagcagcagcagcagtgaccaggtgtccacgccgttctggagctgcgccgtccgggccgcgatctcctgctccggggtctgcaccttcgcggtctcgggcaccggaccgaccgcgccgcgcaccgtcaccgtgtagatgccgagggagggatcgtcggcccagcgcatgagctgcggccaggtgacgtacaccgacgcctgcgccacaggagaaggcgcccgcacgatgccgaccacggtgaagtcggctgccgtggcacgctcacctatccggatgcggtcgccgacggcgacgtcccagttctgggcgtcccacaggtccaccacggcctcgcccttgcgctcggggaaacggcccgaggtgagctgctgccagcgcaggtccttggactcggcgaccggccccacgcccatctcggggtaggaccggtcacccgcgcgcaccgtcagcatggccctgccgagcggtgacgcgttcgcgccatgacgctcgacgagctcgaacgcatcctcgttcgtcagcttggacaccacgtggtcggagttgcggaacggcgccccgaagccggccatgatgccgctctgcgccccggaggtgagcacgccgaccccgacgacgaaggccacggcgacggtgaccgcgatcgccgccgcgacgtacctgcggacatgggtgcgcagcgacgcgagaaagacggtgcgcatcaggcgatccgtccgtcttccagggtgaccacgccgtcggcgtaggccgcggcctcacgctcgtgggtgaccatcacgacggtctggcccagctcgcgggtggatttgtgcaggtaccccaggacctccgccgaggtggtgctgtcgaggtttccggtgggctcgtcggcgaacagcagatccggcccggtgatcagagcccgggcgatggccacccgctgctgctggccgccggacatctcggagggccggtggccgagccggtcggccatgccgagggtttcggcgagcacgtgcacgcgctcggtcgccgcgtcgtcgatgcgccggccgccgagctcgagcgggagcgtgatgttctggaacgccgtgagcatcggcagcaggttgaaagactggaacacgaagccgatgtgctcacggcggaagaccgtgagctcgttgtcgtcgagtgatccgagatcggtgccggccacggtgacagtgccatcgctcgcctgatcgagcccggccaggcagtgcatcaacgtggacttgcccgatccgctcgaccccatgatcgcggtgaacttgccgcgcgggaggtcgaggtcgacgccgcgcagggcatgcacgcgggtttcaccctggccgtacaccttggtcaggtttcgcgcgctggccgccacggtttccagagcggctcgctggccggtcatatagaagcacccttcgattgtgcttgcgtacagtcggcatgcatgagcagaaagccatcattgacggcttcatggcgctattcttcgcgccaaggctggtagtcgtgctggtactccgcaaagcgccacccatcgtagacgagcagtcggccgggcaccaacggttccaaattgaagcagggcgccgtgcggagtaatagtcaagactgtggatgccgagttccttggcgactgtgggaagggtgcttgcaccggacgggcggcattccctggtcagcccccgggtgtccgggccgctggtccggcgttggcgtcgaaggaactgccgccgtacctggggtcgagcacggatgcggaccacgtggcgggcgcggccagcatggccgcgacgccgatggtgagcccggcgctgaccagcgaactacgacggggcctgaccagccgcgccagcgcgagcgcgacgacggcgaccacgccgagcgcgaccgcgccccacatcgcccacggcagaaaagtggggtagaaggaccacaaccagacggcccaggcgagttcggccacgatcgcgagcggaaatatccacgccatcctgcctccgctccgatacgcccgccagaacattacaatgccgattccggacaaagcggctaccggcggcgcgagtacggccacatatgcgctgtgcgggatgacaaagaccgcgctgtagggcagggcgaaggtgagaagccacacgccccacatcaccattccgccgcgtgccgggtcggtacgctcggcccggcgccaccaccacagcccgcacagcagagccatcagcgcgagcggatacagccaaccggacgcgacgccgaggcggccgccgaacagcttgccccagccgcccccatgctcgatgcctatctcggggatgaccatgccagggcgtggccggggcagctgcgtcgatctcttcggaggcgccgggccgatcaccgagcccatgtagttgggcggcagggcgccgggcagattgatgcccaggcgtccgagaccgttgtacccgaacaccatcgcggcggcgctgctgttcgtggtgccgctgatgtagggccggtcggcggccggagtgacgtggtagagcgtgatccacgacagcgacaccacgagcgtcaccactccggcgatccccaggtgctgcagccgacggcgcagtccgatcggcgcgctcaggagataaccgatcgccagggcgggcaggatcatccacgcctgcaacatcttcgcctggaaacccagcccgacccagacgccggcccagaccagcgaccgcagccgtccttccagcacggcccgctgataggagtcgacggcgagcaccaggcacatgaccagcgccccatcggccatgctgtgcccgaacatggacgcggccacgggggtgatggtgaagacggcggcggcgagcagacctggcaccacgcccgcccatcgccgcacgatccggtacatcaccagcaccgagatcacgccctcgatcacctgcggcaaggcaagggcccaggcgtggaagccgaagatcttgaccgagatggcctgcggcacgaaggccccggcgagcttgtcgagcgtgtaggtcgcctgcacgtcgacggtgccgtacaggaacgccttccagttctcggacatgctcttgacggcgtccgagtatctcggtgcgtagtcgaccagcggcaggttccaggcgtagagcaccgctgccgtggccgcgatgcagagcagcgccggccgggcccaccacggctggccgggcggcgagcgccacaccgcccagcgggggaatctgccggcgggtgcggggtcccggcacgcggacggcggagtcatggtgatgtgcgacatgaggaactcccaggcgtttccttcggcagttccctgcctttactcggctgcgtagcgaatgaccggccaggtggtctcgttatatccgccgtccggtgcggcgttcctggcatgctcatcgagtttggcgaacaggttcttgttcggcccgtcgagcaccgacaactgcgtcgcgtagtgcttcatcgcctggaatttccgcgtccgcgcctcctcatcgacgaaactcagctccggagagccgagccggaggccgtcaggcaattcagccaggtcctgggaatacgccgcgtacgggagatcctgccagagtcgcagcggaatacctcgctcgcgtgcggcgagcagcgtggcatcccgcgtggccttgtggtcggggtgtttcccgatggccacacaggtgagaacgagcgtcggatcgcactccgcgatcatggactcgatgtcctccctgatcgcggcgaccaggtcgtggttgttcgccggcgactgctggcggaccatcgagccttcgttgtggtgcagcagccactggccatccggtgaccttcgatagatggcatcgagaaaacggccatgccgatggcccgcaccgagctgatcgagggcggcgatgtcctcatttcgtcggcgcagcggcgcgtcctcggtcggcgacagaccccagcgtgcgtggaatcgctccgccgccggggaataagggggcgccgcgctgccggcgaacaccgtgaagacggttacttttccaccgtcctgctccgcttgggcgaggctggctccgacggagaggacggcatcatccaaatgaggggagattgctaatatccgggttcggtcggcgtcctgcaacatggttgtcagtctggtgtcggccctgccccgttgcaataaagcggaactggacgggacctcgcacgcgtggagaatttccgggcgggccgggcaccacgatgagtcacccggtcacgtcgcagtcacacgttctctgaccagcctgtcaccgtctgctgcgtacaactggtgtcaacgccgacaccgggcaggagaagatgagtgggaagagcgcagccgcacgacctcgcctcggtcgcatcgaacgacgccttcgccgggctcacgtcggcgaccgcggcctctaggatggcgccagccggcgcagcgcgcgccgcatgagggggaagtggatctctaccgagaggccaccctgcgggcgcgcgtgcgcggtcaacgtggcgtcgtgggcgacggcgatggcgcggacgatggacagcccgaggccgtggtggtcgtcggcgcgggtgcggtcgagccgctggaagggctcgaagaggcggtggacctgctcggggggcaccaccgggccggtgttggcgatggagacgacggccttcccggcctcggcccgggtggagagctccacctggccgccaggcacgttgtagcgcatggcgttgtccagaaggttggtgatgagtcgttcgaccagtgccgggtcgcccgtggtgggggcatgagcgatcccggtcaccaatcgggggtgtgggcaaccggcccggagggacttcccgtcggcccaggaatcggttgccgcgccggccgtggggctgttcccgtcagccctccgatcggcactcacgccggccctggggttgttcccgtcagccccggggccggtcccctccatcgtgcggatcgtgtgctcggcgatctccgccagatccaggggctcgcggtgatcgaggccgccctcgctcttggccagcgtcagcagcgattccagcaggcgcccctgctgccggctgaggtcgagcagccgctccatgatcgatcgcatggacggggtgtcggcgtcccgatgcaggaggctctcctccagcaacgcgtgctccagggtgagcggggtgcgcagctcgtgggccgcgttggcgacgaagcgcttctgcgcgtcgagggcgctgtggagacgttccagcagctcgtccaccgtgtcggcgaggttgcgcagctcgtcgcgcgggccgggcagggcgagccgctcgtggacgttgcgggcggagatccgcttgagcgtggtgttcatcgtccgtagcgggcgcagcatcctgcctgccaccagccagccgagcaagaacgagatgaccgtcatcagggccagcgcgatcagcgattggaacagcaggttctccagaatggccgcctgctgctgccgcgcgaaggcgcggaacctgccgccagggtcaccgtccaccagcacgaagggtctggagccgcggaagagcaggtaggtgatggcgagcaggaccacccctgaggcggcgaacaacgcgccgtagacgagcgtgaggcgcagccgtacgctgcggaaggctgttgtcaggcgtcggagacgatgggccactgttccgatggtatgagaccaggaccggccggaggagcccacggcgccgacgatcgcggccccgggccgaggccgagatgccgcgagtcgggcgcctaccgagtcagcgcaccgcgcaccgctgccgtgacctcgggcgccagggtgcgggcgaaggtggtggtgaggtggctgcggtcggagtaggcgatcaggccgccgatgacggggccgcaccgctcgccgcacaccaggtgatcgacactcgccacggagacgaggccggtgtcgtcggcgcgggcggcggcggcgagcggatccggccggagcacgacgccggccgggccgccgcaggagtccagatcgtccgggtgcttggcgatgcagtggggcacgctgtccggcatggccggggtgtcacgcaggacgagcaccggaaggccggcgccggtgaaggcccggagcgtgtcgcggtaggcccgctcggccgcggcctgctggccggctggcgagacgccggcgagcggcacatgcgtacggttggacatgatcaccaggtcgtagccgccgttcacgatggacccgaccgcccacttgttgatcttctggcagttctccgagacccccgcgccttcgaggacgagcggctgatcgacggtgtagcacgccagctgtacgtaggtggtgagctgccagcgctcgctccacagcgccttctccagggccgggacccagtgtcccgcgtgggagttcccgaccagggcgatgcgcctgccggcggcgtcaggtccgtacgtgcacgtgttccgggcgatgaacggttccttgttcacgcacccgtccgcgtacacggcgggcttgtccttcaacgcgacctgaggaggcatcagcaggcccagatcctggcacgccgggtcgcgcacgacgccggcacccaggcatgaccctgcccgcgaggccgcggcctcgaacgcggcactctccgtacgctcggcggcgtcggcgtaggcgacgacgcccgctcccgctcctgcgacgaccaccacgcacgacgcgagcatcgcgaacgtgagcctgcggctgcggaccaggaccgggtgccagcgcagccggtcctcgacgaggtactgcgagagcgcggcgaggaccagggtcagcgcgatcacgcccacggactcgatcacggtcagcgagcggcccagcgcgtacgggaggatcatgatcggcggccaatgccacaggtacaccgcgtaggaggcgttgccgagccactggaccggccgccacgccagcgcccgcccgggtccgccgcgcagaccgtccgcggccgctgcgatcaccaggcaggcccccactgtcggcaccagggcggcggctccggggaaggccgtctcggcgtcgaaccggaccacggcccacccgatcatgcccaggccggcccacgcgagcccggcccggaccgcccgcgcgcgcggcatcgcgcgtacggtgagaaccgcggcgagcagaccgccgagcgcgagctcccagaagcgggtcgtcgacacgaagtacgcggcggccggatcggtcgccgtcttctgcaccgaccaggcgagggacgcggccacgaccgcacccgtgaccaccaccgcgctccacctcgtgaagttctccggagggcgacgcccccgtgccacccgggcggccagccaggccgccgaccccagcagcagcggccagccgaggtagaactgttcctcgatggacagcgaccagtagtgctgcgcgggccagtccggctggtcgacgtcgaggtagttcgcctgcgtgagcgcgagtctcaggttctccacgtacaccgtggcggcgatcacctcacgcgccgccgtccccagcaccgtgagcggcagccagaccaccgacgcggcgagcgtgaccaggagcgcgaggctcgcggccgggatgaggcggcggacgcggcgtgcccagaagtccagcagtctcccgccgccgtgtcccggctgacgcagcaggtggctcgtgatgaggtacccggagatgacgaagaagacgtctacccccacgtacccgccggtcggcccgccgggccacaggtggaacgcgaccaccgccgccaccgcgatcgcccggaggccctgaatgtccgtgcgggactcggagctccggccgccggcgtgctcgcttcgcggcgcacacgacggggcgtgcggcgtcagcccgcatgcgcaggtcggaagggacatctgttcggtgggtgccgggcgctccatggcaactcccgcgtcatcgaggtgctgcgcagccctcgaaggtcgcacccgcggacgagagcctgcttgatcgcaagcgtgctcaacggactcgatgtctacaagccggtccaggtgaacgcttggtcaccaccaccggtgaacgcgtccaagcgcgcgaactgttcagttggacccactcgtggacatcggctccgctcagcacgattgaggtcgctgacttgcgtgcgcgtgtgagaggagtcccgcatggccatagtgtcgccgttcggaggtttgctgaagggcgacggagaggatgatcccgcgccgtccaggatccgcccggggacgttgcgacgagtgctcggatacttccgcccgcacgtcggcaaggtggcgctcttcgttctcgtcaccgcattggattcgatcttcgtcgtcgcgtctccgttgatgttgaaggacctggtggacaagggggttctggggaacgatctggagctcgtcatcctgctggcgtgcctggccgccggcttcgccgtgatgagcacgctgttgcagctcgtgtcggcctacatctccggccggatcgggcagggggtcagttacgacctgcgggttcaggcccttgaccacgtccagcggctgccgatcgcgttcttcacccggacccagacgggcgtgctggtcggccggctgcacacggagctggtcatgacgcagatggcgttcacccagatgctgacggccgccgccagcgcggtcacggtcctgctggtgctggccgagctgttctacctgtcgtggatcgtcgccctcctcacgctggtgctgatcccggtgttcctggtgccctggtcttacgtgggacggcggatgcagcgctacaccagagggctgatggaggagaacgccggcctggccgggctgctgcaggagcggttcaacgtccagggggcgatgctctccaagctcttcggccgtccggccgaggagatggccgagtacgagagcagggccggccggatccgcgggctcgccgtgagcgtcaccctctacggccggatggcccccgccatcttcgcgctgatggccgcgctcgccacggcgctcgtctacggggtcggcggcgggctcgtgctctcgcaggcgttccagctcggcacgctggtcgccctggccaccctgctcgggcggctgttcgggccgatcacccagctggccagcattcaggagaacgcgctcacggtcctggtgagcttcgagcggatcttcgagctgctcgatctgaagccgctgatcgaggaacgccccgacgcggtcgcgctcaaggccggcaaggcctcggacgtccagttcgagaacgtgtcgttccgctaccccagcgcggacgaggtctcgctgccgtcgctggaacagaacgtgcgcaccgggcaggagcgtggtgaagcgacgccggaggtgctgcgcgacgtgagcctgcacgtgccggccggcaccctcaccgcgctcgtgggcccgtccggcgccgggaagagcaccctcacgcacctggtgtcccggctgtacgacccgacctccggaaccgttcgcgtcggcggacacgacctgcgggacctcaccttcgactcgctgcgcgaaacggtgggggtggtcagccaggacacctacctcttccatgacacgattcgggcgaaccttctctacgcccgccccgacgccaccgaggacgagctggtcgaggcgtgccgaggggcgcagatctgggacctgatcgcatccctgccacgcgggctcgacaccgtcgtgggtgatcgcggttatcgcctgtcaggcggggagaagcaacggctggcgatcgcccggctgctgctgaaggcaccctcggtcgtcgttctcgacgaggccaccgcccacctggactcggagtcggaggccgccgtccagcgggcactgacgacagccctgcgcagccgtacctccctggtgatcgcccaccggttgtccacgatccgcgaggccgaccacattctcgtgatcgacgacgggagggtcagggagcgcgggacgcacgaggagttgctggcggaaggcggtctctacgccgacctgtaccacacgcagttcgccaagtcaggcgtcaacgggacccggccgggacagggcgacggggcggagcccgtgcaagaggtggtcggtggaggggaacgatgagcgccggaacgcgggccacaccgacgacggtgctggacctcttcgcccgccaggtgggccgggcacccgatgcggtggctctggtcgacggggaccgggtcctgacctaccggcggctggacgagctcgccggagcgctgtccgggcgcctgatcggccggggtgtcggccggggtgatcgcgtcgcggtgatgatggaccgctcggcggacctggtggtgacgctgctcgccgtgtggcaggcgggggcggcctacgtgccggtggacgccgcccttcccgcccggcgggtggcgttcatggtggcggactccggagcctgcctgatggtgtgctcggaggcgacgcgcgatgcggtaccgcaaggggtcgagtcgatcgcgttgaccggcgagggcggatgcggcacgtcggcggtcacggtggacccgggggatctggcgtacgtgatgtacacgtccggctcgacgggcaccccgaagggggtggccgtcccgcatcggagcgtcgcggagctgacgggaaaccccggctggggggtggagcccggcgaggcggtgctcatgcacgcgccctacaccttcgacgcctccctgttcgagatctgggtgccgctcgtgtcgggcgcccgggtggtgatcgccgcaccgggtgcggtggacgcccggcgcctgcgcgaggcggtcgccgccggggtgacgagggtgcacctgaccgcgggcagcttccgcgcggtggcggaggagtcgccggagtcgttcgcgcacttccgtgaggtgctgaccggtggtgacgtggtgcccgcgtacgcagtgcagaaggtgcgggcggcctgccctcacgtgcggatccggcatctgtacggcccgacggagacgaccctgtgcgcgacgtggcagctgctggagccgggtgacgtcgtggggcccgtcctgccgatcggccgcccgctgccgggccgccgggcctgggtgctcgacgcgtcattgcggccggtggagcccggggtggtcggtgacctgtacctttccggcgccggtctggcggacggctacctggaccgggcggggctgacggcggaacggttcgtggcggatccgtccgcggcggggaggcggatgtatcggacgggggatctggctcagtggaccgcggacggtgagctgctgttcgcgggccgggccgacgaccaggtgaaggtccgcggattccggatcgagccgggcgaggtcgaggccgcgctgaccgctcagccgcacgtccgcgaggccgtggtggtggcgatcgacgggcgcctgatcggttacgtggtggcggacggggacgtggatcccgtactgatgcgccggcggctggcggcgtcgctgccggagtacatgatcccggccgctctggtcacgctggacgcgttgccgctgaccggcagcggcaaggtggacaggagggcgctgcccgagcccgatttcgcgtcggccgcgccgcgccgcgaacccggcaccgagccggagcgcgtcctgtgcgacctgttcgcggagcttctgcaaccggagggaaggggggtaggggtcgatgacggtttcgtcgagctgggcggggactcgatcgtcgcgatccggctggcagcacgtgcgtccagggtggggctgctggtgacgcccgcccagatcttcaaggagaagactccggcacggctggcagccgtcgcgggtgccgtaccggccggcagacccgccgacggcccgctgatcaccctcacggcggaggaggaggcggagctggcgaccgccgtcccgggggccgaggaggtctggccactcgcaccgctccaggaagggctctacttccaggccaccctcgacgacgagggtcacgacatctaccaggcgcaatggatcctggagctggcggggccgctggacgccgcccggctgcgggcctcgtgggaagcggtcttcgcccggcaccccgagcttcgcgtgagcttccaccggcgcgcgtcaggcacgatgctgcaggtcgtcgccgggcacgtcgtcctgccgtggcgagaggtggatctggcggatgcgggcgatatcgacgcggccgtggcggccctgatcagtgaggaacaggagcagcggttcgacctcgccaaggcaccgctgttccggctggtgctggtccgtcacggcgaggaccggcaccgcctgctggtcgtccatcaccacatcctgaccgatggctggtcggtggcggtcatcctcaacgaggtggctgaggcgtacacgaacggcggccgtctcccggaccgcacgggcgcggcctcctaccgggactacctggcctggctggaccggcaggacaaggacgccgcacgtgccgcctggcaggcggagctgtccggcctcgaagggcccgcgccgatcgcgaaggccgccaccacgaccggcgccgggacgggctacgaatatcgcatcgccttcctgacccctgacctccacacgcggctgacggagctggcccgcgaccacgggctgacgctgaacaccctggcacagggcgcatgggcgatggtgctggcgcggctcgcgcggcgcactgacgtggtcttcggcaccacggtcgcctgccgtcccgccgagctccccgaggtggagtcggtgccgggtctcatgatgaacacggttccggtccgggtgccgctgcagggcgcgcaatcggtcgtggacctgctcaccggcctgcaggaacggcaggcggccttgctgccgcaccagcatctggggctgacggagatccagcgggcggcaggacctggcgcgacgttcgacacgctgctggtcttcgagaactacccgcgggacttcgccggccagttcacctacctgggcacgatcgaggggacccactacccgctgaccctcggcatcatcccgggggatcacttcaggatccagctcgtctaccggcgcgggcaggtcggggagagcgtcgccgagtcgatcctgggatggttcaccggcgctctcatgacgatggccgctgatccgcacgggccggtgggccggatcggtgtgggtgaggcccgggccggcggctcggaccgggcgatggcggcgggggagccgctgccggtgctgctacggcgggtggtgaaggaccggccggacgaggtggcggtggtggacggcgacggtgagctgtcgttcggggaattgtgggaacgggcgacggcgctggcggccgagctgagggctcacgggatcgggccggagagccgggtggccgtgatggtgggcaggtcggcgtggtgggcggtcggggtgctgggcgtctgcttggcgggcggcgcgttcatgccggtggatccggcgtatccggctgagcgcgtcaggtggatcctggccgactccgacccacggctggtgctgtgcgcggggacgacgcgggaggcggtgccggaggagttcgcagaccggctggtggtggtcgacgagctggacctcgcggggagcgacgatgcgggcttgccacgggtgagcccggatgacgcggcttatgtgatctatacgtcgggatcgacggggactcccaagggggtcgtcgtctcgcacgcgggcctcgggaatctggcgatggcgcagatcgaccggttcgccgtgtcgccgtcgtcgcgagtcctgcagttcgcggcgctgggcttcgacgcgatggtgtcggagatgttgatggcgctgttgtcgggggcgaggctggtgatggcgccggagccggccctgccaccgcgggtgtcgctggccgaggcgttgcggcggtgggaggtcacgcacgtcacggttccgccgtcggtgctggccaccgccgatgcgctgccggccgggctggagacggtggtggtggcgggggaggcctgcccgccgggcctggccgaacgctggtcggcgggacggcggctggtcaacgcgtacgggccgaccgaggccacggtctgcgcggcgatgagcaggccgttgacgggcagccgggaggtggtcccgatcgggacacccatcgccggcggccgttgctacgtgctggacgcgttcctgcggccgttgccgccggggatcaccggtgagctgtacgtggccgggatcgggttggcgcgcggctatctgggtcgtgcgtcgttgacggctgagcggttcgtggcggatccgttcgtggctggtgagcggatgtatcggacgggggatctggcgtattggacgggtgagggcgagctggtgttcgccgggcgggatgacgaccaggtgaagatccgtgggtatcgggtggagccgggtgaggtggaggcggtgctggcggggcagccgggggtggatcaggcggtggtggtggcgcgtgaggggcggttgctgggttatgtcgtctccggtggtggggtggatccggtgcggttgcgtgagggggtcgcgcgggtgttgccggagtacatggtgccggcggcggtggtggtgctgggtgcggtgccggtgacggcgaacgggaaggtggatcgggaggcgttgccggatccgggcttcggcgggcgggtttcgggccgggagccgcgtacggaggtcgagcgggcgttgtgcgggctcttcgccgaggtgctcgggctgccgggggtgacggcggtggggccggacgacagcttcttcgagctgggcggggactcgatcacttcgatgcagctggcgtcgcgggctcgccgcgaggggatgctcttcggcgcgcgggaggtgttcgagcgcaagacgcctgcggggctcgcggcgatcgtcgatgtgggcggcgagcttgcggcaggtccggccgacggcgtgggggagatcgcgtggacgccgatcatgcgggcgctcggggacgggatcgtggggtcgcggttcgcccagtgggtggtgctgggtgcgccgccggacctacgggcggacgtggtggccgcgggattggcggcggtggtggacacgcacgacgtgttgcggctgcgggtcgtcgatgaccgggcgggccgccggctggcagtgggcgagcgcgggtcggtggacacggccgggctggtcacgcggctcgagtgcggcggccgtccgccggacgaggtcgtggagcgcgcggtgcgggaggccgtggggcggttggacccggtggcgggtgtgatggcgcaggcggtctgggtggatgcggggccggcgcggacggggcggttggtcgtcgtggtgcatcatctggcggtcgacgggatgtcgtggcggatcctggtgcccgacctgcggctggcgtgtgaggcggtggccgaggggcgggatccggtgctggagccggtgtgggggtcgttccggcgctgggcggctctgctggaggagtcggcgctctcgcgggagcgggtcggggagctgcacacgtggcggacgatcgtcgatcaggaggatcggccggtcggccggcggcggctgagcgcaggggatgcggccgggggcgtgcgttcacggtcgtgggtgatgtcgggggatgaggcgtcgctcctggtggggaaggttccggtggcgttccactgcggggtccacgaggtcctgctggcgggcctggcgggagcggtggcgcgctggcacggtgacgacggggtcctggtggatgtggaaggccacgggcgtcatccggccgaggggatggatctgtccaggacggtgggctggttcaccagcatgcatccggtgcgcctggatgtggcggggatcgagctggcggcggtgccggccggtggccgtgcggccgggcagttgctgaaggcggtcaaggagcagtcgcgggcggcgcccggcgacgggctcggttacgggttgctgcgccatctcaatcccgagacgggccccgttctggcggccctgccgtcaccgcagatcgggttcaactacatgggccggttcgtcaccgtcgaccagggcggtgcgcggccgtggcagccggtcggggggatcggcggttcgctggaccccggcatgggcctgccgcatgcgctggaggtcaatgcgatcgtccacgacaggctggcgggcccggagctggtgctcacggtggactggcgggacgacctgctggaggagaccgacatcgaacgactgtgccaggtgtggctggacatgttgtccggattgtcccgccaagcggaggatccttccgcaggcggacacaccgcgtccgacttcgccctactcgacctcgaccaggacgagatcgagggcttcgaagccatagcagcggaactctctggaggccagacatcgtgaacacgccgagcacacccgccggatcggcgcttgaggaagtctggccgctgtcaccgatgcaggaggggatcctctatcacgccgcactcgatgaggcccctgacctctacctcatccagcagtcgcagatcatcgaaggacccttggacaccgagcggttccgcctggcttgggagagcctcctcaaccggcatgcggcgcttcgcgcgtgcttccaccggcggaagtccggtgagtcggtccagctcatcccccgtaaggtgccgctcccgtggtccgagcgcgacctgtccggcctgtccgaggaggacgcgctggccgaggcgagcgtgatcgcggagaaggagcgcgccacgagattcgacccggccaagcctccgctgctgcggcaggtgctgatccggttcggtccggacaagcactgtctggtgacgacgagccatcacctggtcatggacgggtggtcgcgggcgatcctcgagtcggagctcctcgagctctacgccgcgggtggcgccgagccggggctgcggcccgccggctcctaccgggactatctggcctggctggagcggcaggacaaggaggccgcccgcgcggcatggcgtgcggagctggcgggcgccgaccgttcgacactcggcatccccgaagcgtccaggaagacccaggggcagcgggtgcgggaggtgctcggctacgcgccggacttcacctccgctctggtggacttcgcccgccgccatgggctgacgctgaacacgctggtgcagggggcgtgggcgttggtgctggcccggctcacgcgccgtcgtgacgtggtgttcggcgcggtggtctcgggacgtccggcggaggtgcccggcgtggagcaggccgtcgggctgttcatcaacaccgtgccggtgcgcgtccggttggacggcgggcagccggtcatccagctgctgacggagctgcaggagcggcagtccacgctcatctcgcatcagcatctcgggctgcaggagatccagaagctctccggggtgagcttcgacaccgtcgtgtcgttcgagaactacgtcgatccgggggcgggtccgggctccgatcgcgagctgcgcctgagactgaaggagtttcaccagtcggcgccgtacgcgctcctcctcggcatcatgccaggtgagagcctccagaccgacgtggagtaccggcccgagctgctcgacgcccgcgtcgccaaggaggccctccacgggctcgcccgcgtcctcgagcggatgatcgccgagccggagaccgcagtgggccgcctggacgtggtcggtgacgcggggcgcgagctggtggtcgagcggtggaacgagacgggcgacgcgatcggtgcgccgtccgcggtggacctgttccggcgccaggttgcacgggcacccgccgcgacggcggtgacggccggggacctggcctggtcgtacgcggagctcgacgagcggtccggccggctggcgcgggcactgacggaacgcggcgtgcgacgcggcgaccgggtgggcgtggtgctggggcggtcggcagaggtgctggcagcctggctcggagtgtggaaggcaggcgcggcgttcgtgccggtcgacccggactacccggcggaccgggtggcgttcatgctggccgactccgccgtcgcgatggtggtgtgccaggaggcgacctcgggtgtggtgcccccgggctaccagcagctcctggtgaacgacgccgacgacggcgaggccgccctggtcccgatcggggcggacgatctcgcctacgtgatgtacacctccggatcgaccgggaccccgaagggcgtggcgatcccgcacggcggcgtggcggcgctggcgggagatccgggatggggcgtcggacccggcgacgcggtgctgatgcacgccccgcacaccttcgacgcgtcgttgtacgacgtgtgggtgccgctcgtctccggcgcgcgggtcatgatcaccgagccgggggtcgtcgacgcggagcggctcgccgggcatgtggccgacggcctcaccgcggtcaacttcaccgcggggcacttccgcgcgctggcgcaggagtcgccggagtcgttctccgggctgcgcgaggtggcggcgggtggcgacgtggtgccgctcgatgtggtggagcgggtacggcgggcgtgcccgcggctccgggtctggcacacctacggcccgaccgagaccacgctgtgcgcgacgtggaaggcgatcgagcccggtgacgaggtggggccggtgctgcccatcggccgggcactgccgggccggcggctgtacgtgctggacgcgttcctgcggccgttgccgccgggcatcgcgggtgatctctacctcgcaggcgccggagtggcccacggctatctgggtcgtgcgtcgttgacggctgagcggttcgtggcggatccgttcgtggctggtgagcggatgtatcggacgggggatctggcgtattggacgggtgagggcgagctggtgttcgccgggcgggatgacgaccaggtgaagatccgtgggtatcgggtggagccgggtgaggtggaggcggtgctggcggggcagccgggggtggatcaggcggtggtggtggcgcgtgaggggcggttgctgggttatgtcgtctccggtggtggggtggatccggtgcggttgcgtgagggggtcgcgcgggtgttgccggagtacatggtgccggcggcggtggtggtgctgggtgcggtgccggtgacggcgaacgggaaggtggatcgggaggcgttgccggatccgggcttcggcgggcgggtttcgggccgggagccgcgtacggaggtcgagcgggcgttgtgcgggctcttcgccgaggtgctcgggctgccgggggtgacggcggtggggccggacgacagcttcttcgagctgggcggggactccatccattcggtgaagctggcagcgcgggccacgcgtgccggcatgcccttcaccgtggtcgaggtgttcgagcacaagacgcctgcggggctcgcgacgatcgtcgacgtgggcggcgagcccgcggcaggtccggctgatcccccatcggactccgacctgctcggcctggcgcaggacgagatagcggagttcgaggccgaattcgacgacgaacgtcattctctgcgctgatcgaaagcgggcgccgcgcacggtgtgccggcagcctgcgagttgtccaacatcctgtcgtgccaatgacgtatgcccatgagtaggttggctcaatgataagcaaagcaatgcatggaccgattcggcccgcccgcgcggataccctgctggcctcggtaggcgagcgaggcattctgtgtgacttttacgacgagaacgcctcggaaatcttccgtgatttggaggcggacgcgggcggcacggaagaagcccacgggttcgcggcgctcgtccgcccggagtcgggggcgatcctggagctcggggccggaacaggcaggctgacgattccgctcctggagctcggctgggaggtgaccgccctcgaactgtcgaccgcgatgctcaccaccctgcggacgcggctggcggacgcgccggcggacctccgggatcggtgcaccctcgttcacgcggacatgaccgccttcaaactgggagaacgcttcggaacggcgattctcagcccgtccacgatcgacctcctggacgatgccgacagaccagggctgtactcgtcggtccgtgagcatctgcggcccggcgggagattcctgctcggcatggccaaccccgacgcgtccggcaggcaggagccgctggagcgcacccaggagttcacgggcaggagcggccgccgatacgtgctgcacgccaaggtctacccgtcggaggagatccgcgacgtgaccattcatcctgcggatgaatcggcggaccccttcgtcatctgcgtcaatcgcttcagagtcatcaccccggatcagatagcacgagagctggagcaagccggattcgacgtggtcgcgcggaccccactgcccggggtgcgtaatcacgaactggtgctggaagcgcaatggggcagcgtggaagacgcgcattagagccctccggggaaagcgcttgtgtacttttctgcagtcattcgacagtgaggaacagaaatgagtgaggagctcctcttcctccggcccgacaccattatcgaaccgctggccaaccggttctacgcctcgatgtacgcgacggctcccgtcacggccgccatgaatctcgccttccgtaacctgccgatgctggagtcctacctcgcatccccggaatggcatttcgcagccgctcgcgatccgaagttccgcggcggattcttcgtcaacatcgaggagcagcggaagaacgaggtcgaggcgctgctcgctgcgatccggcgcgacagcgcggacgtgctccggttcgccgaggcgatcgcggaggccgagaagatcatccgcgaggaagcgaccggatacgatctcaggccgctctacccgaagctgcctcccgagctgtcgggtctggtggagatcgcctatgacaccggcaacgcggcctcgctgcacttcctggagccgctcatctacaagagcaaggcctacgccgaggactgccagtccgttcagctctccgtggagaccgggatcgagcggccgttcgtgatgagcaccccgcgactgccctcacccgacgtgctcgagctgaacatcccgttccggcatccgggtctggaggagctcttcctgtccaggatccggcccaccaccctggccgccctccgcgaggcgctggagctcggcgacgcggaagcggcgcggctcgccgacctgctggtcccggagccctcgctcgcctccgaccgccatgtcgcggccggagcccggatccgctactgggggcacgcctgcctgctcatgcagacgcccgacgtggccatcatgacggacccgttcatcagcgcggataccgacgcgaccggccgctacacctacaacgacctgcctgaccgcctcgactacgtcctcatcacgcacgggcattccgaccatctggtgcccgagacgctgcttcaactgcgcggccgggtgggcacgttcgtcgtgccgcgaacctcgcgcggcaacctgtgcgatccttcgctggcgctctatctcagaagcttcgggctgcccgcgatcgaggtggacgatttcgatgagatcgagttccccggcgggaagatcgtctccaccccgttcttcggcgagcacgccgatctcgacatccgggccaagtcgacgtattggatcaacctcggtggcaagtcgatctgggtgggcgcggactcctcaggcctcgatccggttctctaccgccatatccgccggcatctcggcgcggtcaacatcgccttcctcgggatggaatgcgatggcgcgccgctgaactggcagtaccagccgttcatcaccaaggcgttgccgaagaagatgagcgacagccgcaagatgtccggctccaacgcggagcaggcaggtgcgatcgtcaccgagctgggcgccgaggaggcgtacatctacgccatgggggaggagagctggctggggcatgtcatggccaccagctacaacgaggactcctaccagctccagcagatcgccgagttcgaggcatggtgttcccgcaagggtgtgaaggccgctcatctgctcgaccagcatgagtggcactggtcgtcatccaggtgatcgcggtggcccgccggtcggccttcgctcaggcgggcagggccgcggtcgcaagcagctgccgaggccgtgctcgccgaggccgtgctcgccgaggccgtgcccgtgctcgcccaggccgtgcccgtgctcgccgacgccgtgctcgtcgaggccggtgccagagggcgcgtcaccggcctctcagcgcaaccggccgcgtgaaccgcccggcggttcggatcgttcgatatcagggccggatcgacaacgcgtggtggaagtggttacgcgggtcgtaggcggccttcaccttgcgcagccgcgggtggttccccttgtagtagaggtcgtgccacggcacgcccgaggtgttcaagcccggatccgcgaggtcgctgtcggggtaattgatgtacgccccatcgctgacgtcgttcggcaccggcaccccgccggtctcggcgtacacatcggcatagagcttgcggacccacgtcagatgcttggcctcgttgccgggattcgcccaaccggtgatgtagttcaccttgagtatcgcgtcgcgctgcggcagggcggtggccgccgggtcgacggtgttcaccttcccgccgtagccgatcagccagacggcgccgtagtcgatcccgtccatgtgggtcatgttctcgtacacggcctgaatctgccggtcggtcagccgcttgcgcaggtagccggctttcgtcttcgacgccgggcccctgcctcctcgccccggcgtcgaggccagccacctctgttcgatcggctcgggcacctcggccggcgggacgccgtcgatcaccgcctcgatgtgcgcgtcgagcagtctccgggcgtccggccgggtggcgtccacctggatgggcatcatgaagccgctctcacccatgcccgggacctcgttcccgatcatgagctgactccacagcccggtgtacggcgagtcgggcccgctgttccgctcgtaccactccccgtggttgcgcagcagccgggagaacgccgcctccgtcatccccgcccagtcgaaggtcaccgtgctcgtgagcaacgtcgcgggcggcttcggcagcagccgctccggattccggccgacgtcctccggcaccctcatccagtacttcgtgaccaccccgaagttcccgccgccaccgccggtgtgcgcccaccacaggtcgtgatgggggtcgtcgcgctcacgggtcgccacgatcacgcgtgccttcccctgtttgttgacgacgacgacctcgaccgcgtacaagtagtcgaccacggagccgaactgccgtgacagcgggccgtacccgcctccgcagatgtgtccgccgacgccgaccccgccgcagaccccacccggtatggtcacgttccagcccaggtagagcttttcgtacacctctgagagcgtgttgcccggctcgatcaggaacgcgttcatcgacgggtcgtacgcgatctccgtcagcagcgacatgtcgatgatgaccttgacgtcggggttgtcgacgaagtcctcgaaacagtgcccaccgctgcggacggcgacccgcttgccggtgcgcaccgtctcctcgacggcatcggccacctgctgggtggagccgaccaggtggatgtagtcgggctcgccgttgaagcggctgttggcgccacgcagcttcaggttgaggtagcgcgggtcgtccggagtcaccttgaccgggccggccggcggtaagcagcgctcgccccgcagctccggtcgcgtggacgaggcgccggccgacgcggcgtccgctccggtgccgccggtcaccaccgccgccgcgcccccggcaagggaggcactcagcaatctacgtcggttcagttttgtcatggcggcgacgttactatcggttcgattcgatcaactcgctgtctgactggacgtaagcgatctcttcacgccgtggccgtacgtggctgtccatcgcctacagatttccgatctctgaaggtacggtcacctgttgaagaacgcgtccgtcagcgcattccaggtgacgccgccgcgaacgcgcgctcacgggaaaactcgccgtcgaggcgggtgacgacggaatcgtggtgtccaaccacggaggccgtcagttggacggtgccgtcctgagccagtttctgccaaacgactgggaacaccatgggacatccacacgatcaagagcccgacggccaaatccgtcctgcggctcagagcccccgccggttggtcgagatgacgagcacctccgggcggcacctgtatcaccgccaggtgcgattctccgatatcgacgcccacggccacgtcaacaatgtgcgtttcctggaatacctggaggacgcctggatcgccctctatctcgacaatgcgggcccgccgcaggaggaccgcgacggattgcccgccgtggggttcgccgtcgtgcgccacgagatcttctatcggcgcccgctcaggttccggcacgggtcggtgcgggtcgagtcgtgggtgaccaaggtgaacagggtgacctgcgagatggccgcgcagatctgctcggacggggaggtgttcgtcgaagcccgctcgatgatcatggggttcgacacgcacaccgccaagccgcggcgcctcaccctgcacgagcgcacctttctcaagcgttacctgcgctgatgtgacttctccattgccggccgcggctccgggcgttggacgattttgaccgccgagatcggccgagcctaccttcacggtgttcgctgcgaccggaaaggtgaattcaatggccgcgtccgaggtcaagcaagtgctccggagcaagctcaggacatgggggtggatgtatcgatgacgaccagcatcgcgtcggcagaagacctttccgtcctcaccggactgagcgagatcaccacgttcgccggcgtggggacagccgtttccgccacgtcctattcgcaagccgagctgctcgaaatcctcgacatacgcgatcccaggatccgatcgctgttcctgaacagcgcgatcgagcggcgttttctcgcgcttccgccccagggccgggacggggagcgcgtggcggaaccgcagggtgatctcctggacaagcacaaaaagctcgccgtcgatatgggatgccgggccctcgagtcctgcctgaagtcggcgggcgcgacgctctcggatgtccgccacctgtgctgcgtcacctcgaccggttttctcacccccggcctgagcgcactcatcatccgcgagctcgggctcgacccgcattgcagccgcgccgacatcgtcggcatgggatgcaacgcggggctgaacgcgctcaacctggtcgcgggctggtccgcggcgcacccgggcgagctcgccgtcgttctgtgcagcgaggcgtgttccgctgcttacgcactggacggcaccatgcgcaccgcggtggtcaacagcctgttcggcgacggatccgccgccctcgccgtcgtctccggtgacgggcgcgctgccggcccgcgcgtcctgaagttcgcgagctacgtcatcaccgacgcgatcgaggcgatgcgctacgactgggaccgcgaccaggaccggttcagcttcttcctcgatccgcagatcccctacgtggtcggcgcgcacgccgagatcgtcgtcgacaagctgctgtccggtacggggctgcgccgcagcgacatcggccattggctggtgcactccggcggcaagaaggtgatcgacgccatcgtcgtcaacctcggcctgagccggcatgacgtccgccacacgaccgctgtgctccgcgactacgggaacctctccagcggctccttcctcttctcctacgaacggctcgccggcgagggcgtgaccaggcccggagactacggggtgctcatgaccatggggcccggctccacgatcgaaacggcgctgatccaatggtgagtggcagtgacatgaacggcgaactggagctgagcctcgacggcacccaggcgctgaccgcgtcggtcgaggagctgaacggcctctgcgaccgcgccgaggaccatcgagcacccggcccggtcatcgtccacgtcaccggcgtgccgcgccttggctggtcgaaggggctgacggtgggcctggtctccaagtgggagcgggtggtgcgccggttcgaacggctcggccggctcaccgtcgccgtggcgtcaggcgactgcgcgggaccctctctcgacctcctcctcgctgccgacgtgcggatcgccgctccggcgacccggctgctgccctcctgggccggcggcgccgcgtggccggggatggccgtctaccggctcacccagcaggccggtacgggcggcatccggcgggccgtgctgctcggggcacccatcgacgccgaccgcgcgctcgccctcaacctgatcgacgaggtgtccgcggacccggcggcgtccctggccggcctggcgggtgccggggacggcgcggagctggcgattcgcaggcagctgatgttcgaggcgagctcaaccactttcgaggacgcgctcggtgctcacctggccgcggtggaccgggccctacgacgggagaccctctcgtgacgacggattggccggcgctgccgcccagggcgccgctcgccctctggaccctgacggcggaggcccagcgagtcgacgacctgctcgccgggctgccggagcctcctgccagaacctccgcccagcgcgatgccgcggcctcggcactcgacaaggtgaggcggatgcgcgcggactacatggaggcgcacgccgaggagatctacggcgagctcacctccggccgcacccggcacctgcgcatcgacgagctcgtacgggccgccgcccgcgcctaccccggcctggtgcccaccgatgagcagatggcggccgagcgcgcgcggccacaggcggagaaggaagggcgcgagatcgatcagggcatcttcctgcgcggggtcctgcgtgccccgaaggcgggcccgcacctgctcgacgccatgctccggcccacccccagggcccttgagctgctccctgaattcatcgagtccggcgaggtgcggatggaggcggtcctgctgcggcgccgtgacggtgtcgcgtacctgaccctgtgccgggacgactgcctcaacgccgaggacgcgcagcaggtggacgacatggagaccgcagtcgacctggcgctgctcgacccccaggtccgggtggggctcctgcggggcggcgagatgagccatccccggtaccgggggcgccgggtgttctgcgcgggcgtcaacctcaagaagctgagctcgggcgacatctccctcgtcgacttcctcctacggcgcgagctgggctacatccacaagatcgttcgcggcgtgtacacggacggttcgtggcactcgaagctgaccgacaagccctggatggcggtcgtcgactccttcgccatcggcggtggggctcagctcctcctggtcttcgaccaggtgctggcggcgtccgactcctacatcagcctgcctgcggcgacggaggggatcattccgggggtcgcgaactaccggctcacccggttcaccgggccacgcgcggcccggcagatgatcctcggcgggcggcggatccgggcggacgagccggacgcacggttgatgatcgacgaggtcgtcccgccggaggagatggacgcggcgatcgatcgcgcactggcccgcctcgacggagatgcggtgccggccaaccggcgcatgctgaacctggccgaggagccgcccgaggcgttcggccggtacctggccgagttcgccctgcagcaggcactgcgcatctacggcagggacgtcatcggcaaggtcggcaggttcgcagcgggatcggcatgagcgagcctcgcgtgcgctacgagaagaaggaacacgtcgcccatgtgacgatgaaccggccccacgtgctgaacgcgatggatcgccggatgcacgaggagctcgccgagatctgggacgacgtcgaggccgacgacgacgtcaggacggtcgtcctgaccggtgcgggaacgcgggccttctccgtcggccaggacctcaaggaacgcgcgctgctggacgaggcgggcacgcaggcctcgacgttcggcagccgggggcaggcaggtcatccccggctgaccgaccgcttcaccttgtccaagccggtggtcgcccgggtgcacggctacgcgctgggtggcggcttcgagctggtgctcgcctgcgacctcgtcatcgcctccgaggaggcggtgttcggcctgccggaggtccggctcggcctgatccccggggcgggaggcgtgttccggctgccgcggcagctgccgcagaaggtggcgatgggccatctgctgaccgggcgccggatggatgcggccacggcgttccggtacggattggtgaacgaggtcgtaccgcttgatgagctggatcggtgcgtggccggatggaccgacgacctcgtacgcgccgctccgctgtctgttcgcgcgatcaaggaggccgccatgcggtcgctcgacattcccctggaggaggcgttcaccacgtcctacccatgggaagagcgtcgtcggcgtagcggcgatgcgatcgagggcgtccgggcgttcgtcgagaagagggacccggtctggacgtcgagatgatccccccgcacacgttgctggtcttcttcgttcaggctgcggccctcctgctgctcgcgttgctcctgggccgcctggccgtacggctgggcctggcggcggtcgtcggcgaactgtgtgccggcgtcatcctcggcccctccgtgctggggcaggtcgcgcccggggcggagcagtggctgtttccctcgccgtcgtcacacatgctggacgccgtcgggcagctcggcgtgttgttgctgatcggcttgacgggcgcgcatctggatctgcggctgatccggcggcagggcgccacggcggtgcgggtgagcgccttcgggttggtcgtgccgatggccctcggcatcggcgccggcctgttgctgccggccgagttccgcgggaccggcggctcggccgtcttcgccctgttcctgggggtgacgatgtgtgtcagctcgatccccgtgatcgccaagacgctgatggacatgaacctgctccatcgcaacgtcggccagctcacgctgaccgccggcatgatcgacgacgccttcgggtgggtgctgctttcggtggtgacggcgatggccaccgccggagccggtgcggggaccgtggtgctgtcgatcgcgtcgctgctcggggtgatcgtcttcagcgtcgtcatcggcaggccggcggtccgggtggcgttgcggacgacggaggatcagggggtgatcgccggccaggtcgtggtgctggtgctcgcggccgcggccgggacgcatgcgctgggcctcgaaccgatcttcggggccttcgtcgccgggctgctggtgagcacggccatgccgaatccggtcagactggcaccgctgcgcacggtgacgctcggggtgctggctcccctctatttcgccaccatgggcctgcgcgtcgatctcacggccctggcgcggccggaggtgctcgccgtggggctgctggtcctggccctggcgatcatcggcaagttcctgggcgccttcctgggcgcctggaccagccggctcagccgatgggaggccttggcgctgggggcggggatgaacgcccgtggcgtcatccagatgatcgtggcgacggtcggcctgcggctgggggtgatcactgacgagatcttcacgatcatcatcgtggtggcggtgatcacctctctgctcgccccgccactcctgcgcctggccatgaccaggatcgaggccaccgccgaggaggaggcccgcctcctcgcctacgggctgcgccccggcgaggcccgggaagacgtacggtgacgacggctcgggatcgtcgtgcccgacgacaaggccggcagccggacggtggtggccggtgccggctcagccacagtgggccggggtcgcgatgcccagccgcgcgtgcaggtgcgcccacagagcagcctgctcgtgccccaggaagaagtggcctccgggcaggacgtggcaggagaactcccgagccgtcaactcggcccatcgcgcgaccgcgtcgagccgtacgacgggatcgtccgcaccggtgaacgccgtgatcggcaccgtcaggggcggcccaggcgtgtggcggtaggactggacgagctggaagtcgttgcgcacgtaggggagggcgaacgcccggaactccgcgctcgcgagcgcctcggcatcggtgccgcccaacaggcgcagcctgtcgatgagcgcctcctcggaggccggcgccacccgatgcgcgagacggccacggtcgtgcgcggccacacctccggagacgaagagatgagccggcgggataccggacccggtgagaagccgcgccgtctcgtaagcgatcaaactgcccatactgtgcccgaacagcgccaccggccggtcgaggagcggcctcagctcacgccccaccgactccgcgagccggtgggcatcaccgacgaggggttcgtgcaaccggtcggcgcggcccggatactgaaccgcgtgcacttctatctccggcgcggccagccggtgccaattccggtagaagaccgccgaaccgcccgcgtgcggaaaacagatcagccgcatcgtggcgagcggccgcctgtcgaaacaccgaaaccaggtggacatgtagcctcgcttcggcctcatatcatggtcttgggtcaatcctggtgacctgactatatgcctgcaccgccataaagtatgtccgtccactcatcggcgggcatgcggcacgagtctgcccaggtcgcacttgacgcctggtcggcaaagggaaaacccttgcttccatggactcccacgttctcgcccatcaattgagcaaggaaacgctgcacggatcgctgatggacccggccatcgagtcgatgaatctactgaacgagattgccggcaactaccccgacgccatttccatggccgcgggccggccgtacgaggagttcttcgacgtcggcctcatccacgactatctggaggcctaccgcgaccatctccgcaacgaccggcggatggatgacgccgggatcagccgcatgcttttccaatacgggaccacgaaggggatcatctccgaccttgtcgcccggcacctcgccgaggacgagaacatcgaggccgacccggcctccgtggtcatcactgtgggcttccaggaggccatgttcctggtgcttcgcgcgctgcgagcgaacgagcgggacgtcctgctcgcccccacgcccacctacgtcggcctgaccggagcggcgctgctcaccgacacccctgtctggccggtccagtccaccgacaacggcatcgacctcgaccaccttgagcaccaactgaaacgcgcccaggaccagggcgcccgggtccgggcctgctacgtgaccccgaacttcgccaaccccaccggcaccagcatggacctgcccgcccgccatcgcctcctggaggtcgccgcggcccacggcatcctgatcctggaggacaacgcgtacggactcctcggccaggaccgcctccccacgctgaagtccctcgaccatgcggcgaccgtcgtctacctcggctccttcgccaagaccggcatgcccggcgcccgggtcggctacgtcgtggcggaccagcacgtagcggggggcggctcgctcgccgacgagctcgcgaagctcaagggcatgctcaccgtgaacacctcgcccatcgcccaggcggtgatcgccggcaagctgctgcgccacgacttcagcctggcccgggccaacgcccgcgagaccgccatctaccagcgcaacctccacctcacgctggacgaactcacccgccggctcggcgccgtcccgggagtcacctggaacgcgccgacgggcgggttcttcatcaccgtcaccgtgcccttcgtcgtggatgacgagctgttggaacacgctgcccgcgatcatggcgttttgttcacgccgatgcatcacttctatggtgggaaggatgggttcaaccagcttcggctgtcgatcagcctgctcaacccgcaactgatcgaggagggtgtctcccggcttgccgggctcgtcaccgcatgtctcccctgaaccatgcctggggccttgagtcggacggccgggttgcgtgcggccgggatgaaggtcaaccacaagcgggtggtgcgcgagcacggcctcgccgggcggtggccagcgaccaaggcctcgacaaacgccatcgccgaccctcccgagggaggatcggcggttgaagatctgtgtgccccctgcaggattcgaacctgcgcacccggctccggaggccggtgctctatcccctgagctaaggggg

SEQ ID NO: 2 - protein, 366 aa, Nonomuria
MHESPVCLAEYEEIAAKVLPADVRDFIDGGSGREQTLRANRAAFDRVFLVPRVLQDVSACSTRATLLGHPATMPVAVAPVAYHRLVHPDGELATARAARDAGVPFTVSTLSSVPVEDVTALGGHVWFQLYCLREHAATLGLIRRAEDAGCRALMLTLDVPWMGRRPRDIRNRFRLPPHVRPVHLTANSGTEAHRGASGGSALAAHTAMELSAAVDWSYLETLRAASGLPLVVKGILHPEDARRAADLGIDGIVVSNHGGRQLDGAVASLDALPGVAESVGGRCEIMLDGGVRSGADVLKALALGASGVLVGRPVIWGLAADGERGVRTVLGLLGAEIEDGLGLAGCGDVAAAQALRTTRPGAGFVS

SEQ ID NO: 3 - protein, 356 aa, Nonomuria
MESLPPLAVDYVEMYVADLKVATLPWTDEYRFAVVGTANASDHRSVALRQGRITLVLTQATSDGHPASAYVRTHGDGVADIALRTPDVDVVFTHAVAAGARPVRSPSRHPGPGPACSAAIGGFGDVVHTLVQRDPGDDPGLPVGFSEAPSAAESGADAAELLDIDHFAVCLPTGDLDIITDFYVATLGFSETFKERIEVGTQAMESKVVQSASGAVTLTLIEPDPMAEAGQIDMFLERHAGAGVQHVAFSSSDAVHAVNTLSERGVRFLSTPGSYYDLLESRIQIRGHTVDQLRATGLLADEDHGGQLFQIFTASTHPRETLFFEVIERQGARTFGGANIKALYEAVEVARSQQRA

SEQ ID NO: 4 - protein, 867 aa, Nonomuria
MLFGRDRELKSLTRLLDSTAAGRGGVAVITGPVVGGKTAILHELGMRSIAAGIRLVTARCTPAEQSLDWGVADQILGRGAAERLTARRGGDAVEDVCVSLFQMAEANPILLTIDDVDLADDPSLLAILSMTPLLTDTRMMIAVTICQDRPPAPLPHVAESLLRLPGIELVELPLLPRPAVRQFATEHLGAETADQLADDLYRFSGGSPLLVRALIEDQEAGAPGLVVGDSFMSAVAACVHGCEPEAVRVAEAVAVLGEHATPDAVGELVGIAPPAATRSMGMLERAGLLAGGRFRHEAGRLAVLGRMTSYGRMEILRRAAEILHRRGGPPSAVATRLLEAGWSGEEWAFDVLVEAGRQAFDEGDFVAVMKCLRLALASGWGTPRRLDVKVMLAAAEWRVDPAVAARHVPDLLDATRSGALRGSHGMELFRQLLWYGRFADAAELIDRLRPSVADRDADASLIAMCHVHPVLLDRLPRSARGSMGQTVEDARRILRQAEPTDEAMDSIISALMALLLGGVSEVAASCETLLKEPGVTKAPTWKAIISAIRAETAWRKGDLAGAEAHAQEALTILQPSGWGVAIGAPLSTLLHAQTAMGHLDEAKATVAVPMPRETAETAFGIGYELARAHYHLVTEQPRAAFAGFLACGQAVQRWGSSLSDVVPWRLGAARACLQLGWRRRAADLVTAQIAHTSSGDLRTYGVALRLHAQLSKPAQRQRLLMQSVDALEAAQDRYQLALSLCDLAGTPQLKGGKDEARAYWVRAQELARECNAKPLMRRLAAQHDHGETAPLSGAERRVAVLAARGHTNREIAEALYITRSTVEQHLTRIYRKLHVQTRGDLGNLFAADIADKATATAGREPREAVRL

SEQ ID NO: 5 - protein, 321 aa, Nonomuria
MDPTGVDIATLPVVEIELSRLSSVYSPRTSGEDPEHVETLLSAQGELPPILVHRPTMRVIDGLHRLKVARVRGETTISVRLIDGTESDAFVLAVEANVRHGLPLSLADRKRAAVRIIGTHPQWSDRRVASATGISAGTVADLRRRRGQGGDEARIGRDGRIRPVDSSEGRRLAAELIRSHPDLSLRQVAKQVGISPETVRDVRGRLEHGESPIPDGSRRLRTKPELLRRAEQDFGHVDGRDRQAVLERLKADPALRLTETGRILLRMLSLHSIDGQEWERILRGVPPHWGTVVARCARDHAQIWAAFADRLEGRATDLAAG

SEQ ID NO: 6 - protein, 369 aa, Nonomuria
MTLERTLIVGTGLIGTSAALALREKGVAVYLSDVDAHAVRLARALGAGQEWTGQRVDLALIAVPPPSVGQRLADLQQRRAARAYTDVTSVKVDPIADAERLGCDLTSYVPGHPLAGRERSGPAAARADLFLGRPWALCPRPETGADAVRLARELVSMCGAEPYTVSAGEHDTAVALVSHAPHVAASAVAARLRDGDDVALALAGQGLRDVTRIAAGDPLLWRMILAANALPVAGVLERIAADLAAAASALRSGDLDDVTDLLRRGVDGHGRIPDKHGGPARDYTVIQVVLQDRPGELARLFNAAGLADVNIEDIRLEHSAGLPVGVVEVSVRPEDTGRLTEALRFHGWHVPPVPDGNSRIDRTRAMVSD

SEQ ID NO: 7 - protein, 217 aa, Nonomuria
MRVLVVEDQVDLADSVARVLRREGMAVDVSHDGDDAQERLSVIDYDVVVLDRDIPGVHGDELCAEIAVDDRRTRVLMLTASGTTADRVAGLSLGADDYLPKPFAFAELVARIRALGRRAHPPAPPILVHGDLRLDPAQRVAIRGGMRLPLTTKELAVLEHLLTARGRVVSAEELLERVWDEQADPFTTTVKATINRLRSKLGQPPVIETVPREGYRI

SEQ ID NO: 8 - protein, 196 aa, Nonomuria
MRRSEGDDEPRTLPPRARDRVYTAVTRVLAVLLLPVAFVRQPGRARELACGWALRMRFPAEDLTGLTDGARAAFTAARAEALWRHGQLVGLTSGYRDPRVQQRMFEEEVRRSGSVAAARMFVAPPAESNHVKGMALDVRPHEGARWLEAHGARYDLYRIYDNEWWHFEHRPECGGTPPRRLPHPGAAWASRNGGRV

SEQ ID NO: 9 - protein, 319 aa, Nonomuria
MDAESVRRQLRLGENATAWLSRLEELGPPPEPVRLPQGDEARDLLHRLEVPAPDVEEIVAATPGPDRDPALWWLLERAHHELVRHMGDYKVKVRGGPTLPYETGAAARYFHVYVFLATLPALRRFHATRDIPEATTWETLTQLGESVAIHRRKYGEGGTNMPWWLTLLVRGLVYRLGRLQYNLAVAKDGTPVLGLHIPEVGGPLIPDIYYDSLRRARPFFERHFPEHGARAATGTSWLLDPQLAEYLAEDSHILQLRRGWTLLDSEPQDGDDAILEFVFRYNGQPLEELPQRSTLEKAVVTHLLAGRHWYQRSGRIELP

SEQ ID NO: 10 - protein, 408 aa, Nonomuria
MRVLLSTSGSRGDVEPLLGLAVQLRELGAETRMCAPPDCAERLAEAGVPLVPVGTSMRAKLHGKRPPSLEDVPRLDAEAIATQLDQVLPAAEGCEVMVVSGVLSAAVAVRSVAEKLGIPYVYVFYCPIYVPSPYYPPPPPLGEQPARDVTDNRVLWDRNNQGAYQRFGAALNSRRASIGLPPVDDIFSYGYTDRPFLAADPVLAPLQRTDLDVVQTGAWIMPDERPLPAEVEAFLEAGPPPVHVEFGSGPAPTDAARVAIEAIRAHGHRVIVSRGWAGLAPPDDRSDCLTVGEVNHQVLFGRVAAVVHAGSAGITTAVTRAGAPQVVVPQMTDQPYHAGRVAELGIGVAHDGRVPTVESLSAALTTALAPETRARAIDVAGKIRADGAAVAAKLLLDTAAGAGRNRTE

SEQ ID NO: 11 - protein, 489 aa, Nonomuria
MEEFDVVVAGGGPGGSTVATLVAMQGHRVLLVEKEVFPRYQIGESLLPSTVHGVCRMLGVTDELAAAGFPVKRGGTFRWGARPEPWTFSFSVSPRITGPTTFAYQVERARFDEILLGNARRKGVVVREGCSVTEVIEDGDRVTGLRYVDPDGGEHAVSARFVIDASGNKSRLYSSVGGTRNYSEFFRSLALFGYFEGGKRLAEPYSGNILSVAFDSGWFWYIPLSDTLTSVGAVVRREMAEKIQGDREKALAALIAECPLISEYLAPARRVTTGKYGQLRVRKDYSYHQTKFWRPGMILVGDAACFVDPVFSSGVHLATYSGLLAARSINSVLAGDVEEKIALHEFEARYRREYSVYYEFLLAFYEMNVNEESYFWHAKKVTNNKEYTELESFVDLVGGLSSGETALATSGRIAERSAEFAAAVDQMADGDDSSMVPLFKSQVVKQVMQEGGQEQMRAVLGADAEPEQPLFPGGLVTSPDGMRWLTHHP

SEQ ID NO: 12 - protein, 420 aa, Nonomuria
MRIDSEWSFDPGMDDDIDAGAPVLQPTANYMMRTHCDPHEDMFALRAHGPLVRIGGDAATQLRVDYVWQALGYDVVRRILGDHENFTTRPRWSSAPSIAGEPIPPNLVGQLSVYDPPEHTRLRGMLTPEFTARRIRRLEPAMQDLIDDRIDELEAAGPPADVQALFADPVGGGVLCELLGIPRDDRIEFIRRVRQNVDLSRGFKARAADSAAFNRYLNGLIIRQRKDPDEGFIGMLVREHGDDVTDEELKGVLTALILGGVETVAGSIGFGVLALLDHPDQRQSLFAGREEADRVVGELLRFLSPVQQPNPRLAVRDVVVDGQLIKAGDYVLCSILMANRDEALTPNANVLDVRRDCGSHVGFGHGIHYCIGAAIARTLLRMAYQSLWRRFPGLRLAVSAEEVKFRNAFIDCPDELPVTW

SEQ ID NO: 13 - protein, 398 aa, Nonomuria
MSGDGARPLHTRRQDLDPADELRAAGTLTRITIGSGADAETTWLATGYTVVRQVLGDHRRFSTRRRWNERDEIGGRGNFRPRELVGNLMDYDPPEHTRLRQKLTPGFTLRRIRRLKPYIEQIVTERLDALERAGPPADLVELVADEVPGAVLCELIGVPRDDRAMFMQLCHGHLDASRSQKRRAAAGAAFSRYLLAMIARERKDPGEGLLGAVLAEYGDTATDEELRGFCVQVMLAGDDNISGMIGLGVLALLRHPEQIAALQGDDQSADRAVDELIRYLTVPYAPTPRVAMEDVTIGGQVIKEGETVSCSLPMANRDPALLPDAGRLDVRREPVPHVAFGHGVHHCLGAALARLELRTVYTALWRRFPTLRLADPDREPSFRLTTPAYGLTSLMVAW

SEQ ID NO: 14 - protein, 384 aa, Nonomuria
MVVPLPHQRLRLDPVPALFDLQEDGPLHEYDTEPGLDGHKQWLVTGYGEIREILADANRFSSMRPVEDEAERAWLPGILQSYDAPDHTRLRRTVTRANTARRIESLRPVVEETVEDCLADLESMGSPVDFVRNAAWPIPALIACDFLGVPRDDQAELSRMFRDSRESRVPRQRNVSGLGIVDYARKLAARERLDPGTGMIGGIVREHGGEVTDEELAGLVEGIMIGAVEQMASQLAIAVLLLVTHPDQMALLRERPELADSAAEEVFRYASIVETPSPRTALVDTRLAGRDIHAGDVLTCSILAGNRAREDRFDLTRGNPEHLAFGHGVHFCLGAPLARLQAQVALPALVRRFPSLRLAVPAEDLRFKPGKPAPFAVEELPVEW

SEQ ID NO: 15 - protein, 393 aa, Nonomuria
MEVFEELNVVLPGELHWRDRFDPVPQLRSFMAEGPMTELGAEEGPGGRTAWLATGFDEVRQVLGSDKFSSRLLYGGTAAGIVFPGFITQYDPPEHTRLRRVVSPAFTVRRMERFRPQVDQVVEDCLDAIESIGGPLDFVPHFGWSIATTATCDFLGIPRDDQAELSRSLHASRSQRAASRRGAAGNKFMTYMGQVVARTRRDPGDDMLSVVVREHGDEITDAELTGLAAFVMGAGGDQVARFLAAGAWLMAEVPEQFALLRDKPDVVPDWLEEMVRYLTIDEKLTPRIALEDVRIGDRIVKAGDTVTCSLLGANRRHFPGPDDQFDLTRDRAPNVAFGHGIHHCLGRPLAELIFRSAIPALARRFPALRLAEPEQEIRLGPPPFDVKALLLDW

SEQ ID NO: 16 - protein, 69 aa, Nonomuria
MTNPFENEDGSFLVLVNDEGQHSLWPSFAEVPPGWTRVHGVATRQECLAYVEENWTDIRPKSLIAEAGA

SEQ ID NO: 17 - protein, 1863 aa, Nonomuria
MTIDDTRAKPRSSVEDVWPLSPLQEGMLYHTALDDDGPDTYTVQTVYGIDGPLDAGRLRASWQALVDRHAALRAYFRYVSGAQMVQVIAREAEIPWRETDLHGLPDDLLDSEVDRLAADELAERLPLDAAPLMKLHLIRLGPASHRLVHTLHHVLLDGWSMPILHRELAAIYAAGGDASGLPAAVSYRDYLAWLGRQDKEAARAAWRQELAGLDTPTLVAPADPARVPDMGTAVIELSAELTDGLARLARGHGLTLNTVVQGAWAMVLAQLAGRTDVVFGATASGRPAELAGVESMVGQLLGTLPVRVRLEGGRRVVELLAELQRSQSALMAHQHLGLQEMQAAVGPGAVFDTLVIYENFPRQGLGRAEEDGGLDLRPVRRGRNSSHYPFTLITGPGAQMPLILDYDRGLFDEAAAESVVGALARVLERLVAEPDVLVGRLTLLSEAERALVVEDWNATAGPTPGQSVLDLFGRRVATAPDAVAITDAGGADLTYAEVDQAANRLARHLAARGIGRGDRVGVVMDRSPDLLIAFLASWKAGAAYVPVDVEHPAERIEFVLADSGVSAVLCTRATREVAPADAIVIDAPETRAAIDAGAATAPQIRLSADDLAYVMYTSGSTGLPKGVGVPHGAVAGLAGDEGWRIGPGDAVLMHATHVFDPSLYAMWVPLAMGGRVVLTEPGVLDALGMRQAVERGVTFVHLTAGTFRALAESSPECFAGLVEVGTGGDVVPAQSVEHLRRAVPGLRVRNTYGPTETTLCATWKPIEPGEEVGRELPIGRPMTNRRIYILDAFLRPVAPGVAGELYIAGTGLARGYLGGPGLTAERFVAVPASVDPSPGERMYRTGDLARWNRDGEVVFLGRTDDQVKIRGYRVELGEVEAVLAAQRGVVEAVVVAREDQPGEKRLVGYFISDGTDAGPAEIRREMALVLPAYMVPLAVVALPALPVTPNGKVDRLALPAPDLVGRAPDRAQESETEKVLCALFAEILGVDRVGVDDAFHDLGGSSALAMRLIARIREELGADLPIRQLFSAATPAGVARALAAKSRPALEPAERPGRVPLTAQQLSAWLLASPGEAAGLHVSVALRLRGRLDVPALEAALGDVAARHEILRTTFPGHAQSVHQHVHDASPVDLTPVPATEESLPGLLTELRESVFDLTREVPWRGDLFRLSDGEHVLHLMVHRILADDESLDVFLRDLSAAYGARRAGRAPERAPLTLQFADYAIWERRLLEGERDADGLINEQLVFWRDNLAGIHGETVLPFDRPRSAVASRRAGTVSLRLDAGPHARLVEAVDPIGAHPFQIVHAALAMLLTRLGAGHDLVIGTKLPRDDDLIDLEPMIGPFARPLALRTDLSGDPTFLEVVTRAQEAIRSARQHLDVPFARIVELLDLPVSLSRHPVFQVGLEVHEEDLGAWDATELPALRTSVEPVGPEAIELDLAFRLTERRDEDGIEGTLHYAADLFDQATAESLARRLVSFLEQVAEDPQRRVSDLDVLLDDAERERPAEAPAKWSEAVPPVAADLAEGGPLGALVLDDRLRPAVAVGELYLTGAAVDAEPGDRTLACPFGATGRRMLPTGLLARWTAGGTLVVVGERRGSSGSVKTGTGDFEVLLPLRAGGNRPPLYCVHASGGLSWNYAPLLRSLPPNQPVYGVQARGLARTEPLAAGVEEMAADYVEQIRAVQPTGPYHLLGWSLGGRIAQEMARVLEEQGEQVGLLALLDAYPTDVGRLRRPRGDAADQEAADFDRQQEQQAQLAAAVATEAGARKRLDEVMEHLARVGPLHTSRSFGCDILLFVATVNRPSHLPVADAIASWRPLTTGTVEPHEIEIDHMQMLQPAALARIGAVVAEKLRPRPDGERTQR

SEQ ID NO: 18 - protein, 4083 aa, Nonomuria
MAQSRIEDFWPLSPLQQGLLFHTTYDDDWPGLYVGHWILNLNGPVEADRLRAAWEALLARHAALRACFRQRKSGETVQLIARQVELPWRVVDLSHLSEPEEAVRAVAEEDRTRRFDLAKAPLLRLTLIRLAGDDHRLVMTCHHAIMDGWSMPIMLDELSMLYAADGSPLDLPAVPSYRDYLVWLDRQDKERTLSAWAAELRGVEEPTLVAPADANRAPAMPENITVELPEDLTRALSELARTHGLTLNTVVQGAWALLLAQLAGRTDVVFGAAVSARPPDLPGVEGMVGLFLNTVPVRVRLSGSTPVIEFLADLQKRQSALIPHQYMGLADIQRTAGAGAVFDTLLVFQNFPRELRPSDAAAAFDIRIDQGREAAHYPLTLVAVPGESMLLNLDHVTDLFDREAALAILERFTGILRQLAGAGDLTVAEVDVTSAAERALVVNAWSAAPRVAPGELAPDLFDRQVERGRDRVAVVEGKRAVSFGELAEHAERLAGYLSGRGVRRGDRVAVVMGRSPGLIATLLAVWKAGAAFVPVDPAYPAERVQFMLADAEPAAVVTERACQAAVPAGGLDPIVLDDPDTLRAVAEHARLSAGAHADDLAYVMYTSGSTGRPKGVAVSHGNVAALAGEPGWGLGPEDAVLMHASHAFDISLFELWVPLLSGARVVLAEPGAVDGEALAGYVAGGVTCAHLTAGTFRVLAEESPESVAGLREVLTGGDAVPLAAVERVRRACPDVRVRHLYGPTEATLCATWWLLQPGEPTGPVLPIGRPLAGRRVYVLDAFLRPVPPGVTGELYVAGAGVAQGYLGRPALTAERFVAEPFVPGGRMYRTGDLARWTDQGELAFAGRADDQVKIRGYRVEPGEIEAVLAGLPGVGQAVVSAREERLIGYVVAETGGDLDPVRIREQLAATLPEFMVPAAVLVLDALPLTGNGKVDRRALPEPDFAAGAVDREPATDAERILCGVFAEVLGAGRVGVADSFFELGGDSISSMQVAARARRQGIPLTPRQVFEHRTPERLAALAQQAPGRRASSVEPGVGEIPRTPVMRALGDDAVRPGFAQARVVVTPAGFAPDALVTALQAVLDVHDLLRTRVEPDGRLMVAEPGAVDAAGLVTRVAAGNGNLAERAEREARTAAGTLDPSEGIMVRAVWVDAGDAEPGRLALVVHHLVVDAVSWAILLSDLRAAYDEAVSGGTPVLEPAVTSYRQWARRLAGQALSESTVAEAGHWAGVLEGGDLPLERHPGQSASWSRTLSDAQARNLVARVPAAFHCGVQDVLLAGLAGAVARWRGADAGILVDVEGHGRHAADGEDLLRTVGWFTSVHPVRLDVSGVGPGAAAAGELLKAVKEQARAVPGDGLGYGLLRYLNPETGARLAELPSAQIGFNYLGRSGVASEDTAWQVCEGALGGQAAGPDLVQSHALEVGADVQDTPAGPRLRLAIDGRDLDPAAVERLGEAWLDTLAGLAALADTPGAGGHTPSDFELVEVRQRDVDELEAVAPGLTDVWPLSPLQEGILFERAFDEDGVDVYQTQRILDLDGPLDAQRLHAAWQSVIDRHETLRTGFHQLGSGETVQVVVGEAEVLWREADLSRLDEPDAEVERLLAADQAERFDVSRAPLLRLLLIRLGAARHRLVVTSHHVLVDGWSTPILLGEMLTAYADGRVSPAPPSYRDYVAWLSRQDEDAARSAWRAELAGLDEPTVVGLDAGKAPVMPDGHAEWLSEEATRALTGFARGHGLTLSTVVQGAWALVLARLARRTDVVFGTVVSGRPADALPDVERMVGMFINTVPVRVRLDGAVPVLDLLQDLQRRQSSLTEHQHLGLPEIQKAAGPGSIFDTILMIVNYPLDADGLDDGGVAISSIRTRTGTTYPLSVSVIPGARLQIQLDYRPDWIGGDLAAEITGQVVRVLARMVAEPSLPVGRLAVTSRSTRGSVTERWNSTGAAAGGSSVPELFRRQADAAPDATAVIGDGRTLSYAGLDRESDRLAGHLARRGVRRGDRVGVLMERGADLIVALLAVWKAGAAQVPVNVDYPAERIERMLADAGASVAVCAGATRHAVPDGIEPVVMDAPATEAERHEAPPLAVGAHDVAYVMYTSGSTGVPKGVAVPHGSAAALAGDPGWSQGAGDRVLMHASHAFDASLLEIWVPLVSGACVMVAEPGAIDAQRLRDVIARGATTVHLTAGTFRVLAEESPDSFSGLREVLTGGDVVPLESVARVRRACPEVRVRELYGPTEVTLCATWHLIEPHTETGDTLPIGRPLAGRQVYVLDAFLQPVAPNVTGELYLAGAGLAHGYLGAPAATSERFIAVPASVNPAASGERMYRTGDLARWTDRGELLFAGRADSQVKIRGYRVEPGEIEAALAEVPHVAQAVVVAREDRPGEKRLIAYVTAEEGSGLDPDAVREHLAGRLPEFMVPAAVVLLDGVPLTPNGKIDRAALPVPEFTGKAAGREPRTEAERVLCELFAEVLGVARAGAEDSFFELGGDSILSMRLAARARREELVFGAKDVFERKTPAGIAMVAERGGATRASLDDGVGEVMSTPVIRALLERDPDAMTRGALSQWVTAGAPDDLSVDVLAAGLGAVIDAHDMLRSRIVRTGAAQPRLVVAGRGAVDAATLVERVEAGTGDVDEIADRCARDAAARLDPHAGVMIRAVWVDAGPGRVGRLVVAAHHLVVDVVSWRILLPDLQVACEAVAAGRRPVLDPVDVSFRRWARTLADQAVTRATELETWTEILDGARSRLGELDPARDTVSTAGRTSWTLPHDRAGVLVEQATSAFHCGVHEVLLATLAGAVAHWRGGTAVVVDVEGHGRRPIDELDLSRTVGWFTDVHPLRLDVTGIDPAEVIAGGGAAGHLLKQVKENVRAVPDGGLGYGILRYLNAGTGQALAAAPKPEIGFNYLGRFPSRSAGAPEPWQLLGTIGGTAEQDTALRHAVEIDAAVLDGAAGPELSLTVTWAGRLLGEAEAESLAQAWLAMLTGLAAHVGGGGAGGHTPSDFPLISLTQQDVAEVEAAVPTLLDIWPLSPLQEGLLFHAADERGPDVYAGMRKLALDGPLDVARFRASWQALLDRHPALRASFHQLGSGAAVQAIAREVPLDWQETDLSRLPEDEALAEFDRLAEQLHTERFDLTRAPQLRLHLVRLGERRHRLVLTSHHIVADGWSLPLITEDVLTVYESGGDGRALPAATSYRDYLAWIARQDKAAAREAWRAELAGLDEATHVVPPETITTPLEPERVGFELDEALSRRVVEFTGRHGVTANTLFQGIWALHLARLTGRDDVVFGAAVAGRPPEIPGVESAVGLFMNMLPVRARLAGAEPFLDMLTDLQERQVACMPHQHVGLSEINQLAGPGAAFDTIVVFENYPPPPPRPEGPDALVMRPAGIPNDTGHYPLSMRASVAGRVHGEFIYRPDVVDRAEAEEMLASILRALEQVVAEPRVPVGRVGLIGPEQRRLVVEEWNRTGVPPAAEPVPMLFRRQVERSPDAVAVVDAARSLSYSGLLDEAEELARLLVGLGVRRETRVGVLVGRSAELVVALLGVSSAGGVFVPMDPDYPRERISFILADSAPEVLLCTSETRQAVPEEFAGAVVALDAPLAADPRTALPRVEAGDGAYVIYTSGSTGVPKGVLVPHAGLGNLASAQIERFGVTSASRILQFAALGFDAAVSELCMALLSGGTVVLADAESMPPRVSLGDAVRRWGITHVTVPPSVPAVEDDLPDSLETLVVAGEACPPALVDRWSPGRRMINAYGPTETTVCATMSSPLSPGRDVVPIGRPITGLRAYVLDAFLQPVPPGVTGELYVAGAGLARGYLGRPGLTAERFVAVPASVSPARPGERMYRTGNRARWTRDGELVFTGRADAQVKVRGYRIEPGEIEAVLADHPGVAQVAVVAREDGPGQKYLVAYVVPAAEQVAGAPSEAGQDGALISALRESAAGRLPEHMRPAAFVPLDTMPLTPNGKVDHRALRAPDFARSSSGRDPRSAMEAKLCELFAEVLGLEEVGAGDSFFELGGDSITSMQLSALARRKGLDLTPWQVFDEKTAERLAAVVKELPADGEGTGEPEPPAGTLVDLSPDQLDQLEAGPAGG

SEQ ID NO: 19 - protein, 753 aa, Nonomuria
MAGFGAPFRNSDHVVSKLTNEDAFELVERHGANASPLGRAMLTVRAGDRSYPEMGVGPVAESKDLRWQQLTSGRFPERKGEAVVDLWDAQNWDVAVGDRIRIGERATAADFTVVGIVRAPSPVAQASVYVTWPQLMRWADDPSLGIYTVTVRGAVGPVPETAKVQTPEQEIAARTAQLQNGVDTWSLLLLLFAGIAVFVSILVIANTFSILLAQRMRDFALLRCVGATRRQVVSSVRREAAVVGLLSSLAGVLVGAGLGYGLIALIKTLSPITPIAAPAPPAPWLLGGLAIGLTATLVAAWLPIRRVVRVSPLAALRPDTATDPRTATGRARLVLGVFMLIAGLVLLASAMAWHSTVLMLAGGGSLFTGVLLFGPVLIPRLLEITGTRLGTIGRLATKNAVRNPRRTATTAASLLVGITLITAVLTGVAITSEALNERLDGQHPIDAALVSTGKPFSADFLDKVRGTSGVDQAIAVDGAVATVSGLDKPIPVVTAPDAQRVAHDGGSFARVEPGVLRLDESAFRQLRLRAGDKVRVTVGDRRAVLQVSLATGWGLQAVVAPETLARLTDSAAPRAVWIRASADADSTRLVGELGDLAAAAGANVNDQLEARETENAPLMILTWAIVALLGFSVAIALVGIANTLGLSVLERVREHALLRALGLTRRQLRRMLAAEAVLLSLVAAVLGTVIGIGFAWVGYETFVKQALDNATMQVPWPLLAVVVLVAALAGLLASVLPARRAVRVTPAAGLSFE

SEQ ID NO: 20 - protein, 232 aa, Nonomuria
MTGQRAALETVAASARNLTKVYGQGETRVHALRGVDLDLPRGKFTAIMGSSGSGKSTLMHCLAGLDQASDGTVTVAGTDLGSLDDNELTVFRREHIGFVFQSFNLLPMLTAFQNITLPLELGGRRIDDAATERVHVLAETLGMADRLGHRPSEMSGGQQQRVAIARALITGPDLLFADEPTGNLDSTTSAEVLGYLHKSTRELGQTVVMVTHEREAAAYADGVVTLEDGRIA

SEQ ID NO: 21 - protein, 535 aa, Nonomuria
MSHITMTPPSACRDPAPAGRFPRWAVWRSPPGQPWWARPALLCIAATAAVLYAWNLPLVDYAPRYSDAVKSMSENWKAFLYGTVDVQATYTLDKLAGAFVPQAISVKIFGFHAWALALPQVIEGVISVLVMYRIVRRWAGVVPGLLAAAVFTITPVAASMFGHSMADGALVMCLVLAVDSYQRAVLEGRLRSLVWAGVWVGLGFQAKMLQAWMILPALAIGYLLSAPIGLRRRLQHLGIAGVVTLVVSLSWITLYHVTPAADRPYISGTTNSSAAAMVFGYNGLGRLGINLPGALPPNYMGSVIGPAPPKRSTQLPRPRPGMVIPEIGIEHGGGWGKLFGGRLGVASGWLYPLALMALLCGLWWWRRAERTDPARGGMVMWGVWLLTFALPYSAVFVIPHSAYVAVLAPPVAALSGIGIVMFWRAYRSGGRMAWIFPLAIVAELAWAVWLWSFYPTFLPWAMWGAVALGVVAVVALALARLVRPRRSSLVSAGLTIGVAAMLAAPATWSASVLDPRYGGSSFDANAGPAARTPGG

SEQ ID NO: 22 - protein, 270 aa, Nonomuria
MLQDADRTRILAISPHLDDAVLSVGASLAQAEQDGGKVTVFTVFAGSAAPPYSPAAERFHARWGLSPTEDAPLRRRNEDIAALDQLGAGHRHGRFLDAIYRRSPDGQWLLHHNEGSMVRQQSPANNHDLVAAIREDIESMIAECDPTLVLTCVAIGKHPDHKATRDATLLAARERGIPLRLWQDLPYAAYSQDLAELPDGLRLGSPELSFVDEEARTRKFQAMKHYATQLSVLDGPNKNLFAKLDEHARNAAPDGGYNETTWPVIRYAAE

SEQ ID NO: 23 - protein, 420 aa, Nonomuria
MAHRLRRLTTAFRSVRLRLTLVYGALFAASGVVLLAITYLLFRGSRPFVLVDGDPGGRFRAFARQQQAAILENLLFQSLIALALMTVISFLLGWLVAGRMLRPLRTMNTTLKRISARNVHERLALPGPRDELRNLADTVDELLERLHSALDAQKRFVANAAHELRTPLTLEHALLEESLLHRDADTPSMRSIMERLLDLSRQQGRLLESLLTLAKSEGGLDHREPLDLAEIAEHTIRTMEGTGPGADGNNPRAGVSADRRADGNSPTAGAATDSWADGKSLRAGCPHPRLVTGIAHAPTTGDPALVERLITNLLDNAMRYNVPGGQVELSTRAEAGKAVVSIANTGPVVPPEQVHRLFEPFQRLDRTRADDHHGLGLSIVRAIAVAHDATLTAHARPQGGLSVEIHFPLMRRALRRLAPS

SEQ ID NO: 24 - protein, 709 aa, Nonomuria
MSLPTCACGLTPHAPSCAPRSEHAGGRSSESRTDIQGLRAIAVAAVVAFHLWPGGPTGGYVGVDVFFVISGYLITSHLLRQPGHGGGRLLDFWARRVRRLIPAASLALLVTLAASVVWLPLTVLGTAAREVIAATVYVENLRLALTQANYLDVDQPDWPAQHYWSLSIEEQFYLGWPLLLGSAAWLAARVARGRRPPENFTRWSAVVVTGAVVAASLAWSVQKTATDPAAAYFVSTTRFWELALGGLLAAVLTVRAMPRARAVRAGLAWAGLGMIGWAVVRFDAETAFPGAAALVPTVGACLVIAAAADGLRGGPGRALAWRPVQWLGNASYAVYLWHWPPIMILPYALGRSLTVIESVGVIALTLVLAALSQYLVEDRLRWHPVLVRSRRLTFAMLASCVVVVAGAGAGVVAYADAAERTESAAFEAAASRAGSCLGAGVVRDPACQDLGLLMPPQVALKDKPAVYADGCVNKEPFIARNTCTYGPDAAGRRIALVGNSHAGHWVPALEKALWSERWQLTTYVQLACYTVDQPLVLEGAGVSENCQKINKWAVGSIVNGGYDLVIMSNRTHVPLAGVSPAGQQAAAERAYRDTLRAFTGAGLPVLVLRDTPAMPDSVPHCIAKHPDDLDSCGGPAGVVLRPDPLAAAARADDTGLVSVASVDHLVCGERCGPVIGGLIAYSDRSHLTTTFARTLAPEVTAAVRGALTR

SEQ ID NO: 25 - protein, 648 aa, Nonomuria
MAIVSPFGGLLKGDGEDDPAPSRIRPGTLRRVLGYFRPHVGKVALFVLVTALDSIFVVASPLMLKDLVDKGVLGNDLELVILLACLAAGFAVMSTLLQLVSAYISGRIGQGVSYDLRVQALDHVQRLPIAFFTRTQTGVLVGRLHTELVMTQMAFTQMLTAAASAVTVLLVLAELFYLSWIVALLTLVLIPVFLVPWSYVGRRMQRYTRGLMEENAGLAGLLQERFNVQGAMLSKLFGRPAEEMAEYESRAGRIRGLAVSVTLYGRMAPAIFALMAALATALVYGVGGGLVLSQAFQLGTLVALATLLGRLFGPITQLASIQENALTVLVSFERIFELLDLKPLIEERPDAVALKAGKASDVQFENVSFRYPSADEVSLPSLEQNVRTGQERGEATPEVLRDVSLHVPAGTLTALVGPSGAGKSTLTHLVSRLYDPTSGTVRVGGHDLRDLTFDSLRETVGVVSQDTYLFHDTIRANLLYARPDATEDELVEACRGAQIWDLIASLPRGLDTVVGDRGYRLSGGEKQRLAIARLLLKAPSVVVLDEATAHLDSESEAAVQRALTTALRSRTSLVIAHRLSTIREADHILVIDDGRVRERGTHEELLAEGGLYADLYHTQFAKSGVNGTRPGQGDGAEPVQEVVGGGER

SEQ ID NO: 26 - protein, 2097 aa, Nonomuria
MSAGTRATPTTVLDLFARQVGRAPDAVALVDGDRVLTYRRLDELAGALSGRLIGRGVGRGDRVAVMMDRSADLVVTLLAVWQAGAAYVPVDAALPARRVAFMVADSGACLMVCSEATRDAVPQGVESIALTGEGGCGTSAVTVDPGDLAYVMYTSGSTGTPKGVAVPHRSVAELTGNPGWGVEPGEAVLMHAPYTFDASLFEIWVPLVSGARVVIAAPGAVDARRLREAVAAGVTRVHLTAGSFRAVAEESPESFAHFREVLTGGDVVPAYAVQKVRAACPHVRIRHLYGPTETTLCATWQLLEPGDVVGPVLPIGRPLPGRRAWVLDASLRPVEPGVVGDLYLSGAGLADGYLDRAGLTAERFVADPSAAGRRMYRTGDLAQWTADGELLFAGRADDQVKVRGFRIEPGEVEAALTAQPHVREAVVVAIDGRLIGYVVADGDVDPVLMRRRLAASLPEYMIPAALVTLDALPLTGSGKVDRRALPEPDFASAAPRREPGTEPERVLCDLFAELLQPEGRGVGVDDGFVELGGDSIVAIRLAARASRVGLLVTPAQIFKEKTPARLAAVAGAVPAGRPADGPLITLTAEEEAELATAVPGAEEVWPLAPLQEGLYFQATLDDEGHDIYQAQWILELAGPLDAARLRASWEAVFARHPELRVSFHRRASGTMLQVVAGHVVLPWREVDLADAGDIDAAVAALISEEQEQRFDLAKAPLFRLVLVRHGEDRHRLLVVHHHILTDGWSVAVILNEVAEAYTNGGRLPDRTGAASYRDYLAWLDRQDKDAARAAWQAELSGLEGPAPIAKAATTTGAGTGYEYRIAFLTPDLHTRLTELARDHGLTLNTLAQGAWAMVLARLARRTDVVFGTTVACRPAELPEVESVPGLMMNTVPVRVPLQGAQSVVDLLTGLQERQAALLPHQHLGLTEIQRAAGPGATFDTLLVFENYPRDFAGQFTYLGTIEGTHYPLTLGIIPGDHFRIQLVYRRGQVGESVAESILGWFTGALMTMAADPHGPVGRIGVGEARAGGSDRAMAAGEPLPVLLRRVVKDRPDEVAVVDGDGELSFGELWERATALAAELRAHGIGPESRVAVMVGRSAWWAVGVLGVCLAGGAFMPVDPAYPAERVRWILADSDPRLVLCAGTTREAVPEEFADRLVVVDELDLAGSDDAGLPRVSPDDAAYVIYTSGSTGTPKGVVVSHAGLGNLAMAQIDRFAVSPSSRVLQFAALGFDAMVSEMLMALLSGARLVMAPEPALPPRVSLAEALRRWEVTHVTVPPSVLATADALPAGLETVVVAGEACPPGLAERWSAGRRLVNAYGPTEATVCAAMSRPLTGSREVVPIGTPIAGGRCYVLDAFLRPLPPGITGELYVAGIGLARGYLGRASLTAERFVADPFVAGERMYRTGDLAYWTGEGELVFAGRDDDQVKIRGYRVEPGEVEAVLAGQPGVDQAVVVAREGRLLGYVVSGGGVDPVRLREGVARVLPEYMVPAAVVVLGAVPVTANGKVDREALPDPGFGGRVSGREPRTEVERALCGLFAEVLGLPGVTAVGPDDSFFELGGDSITSMQLASRARREGMLFGAREVFERKTPAGLAAIVDVGGELAAGPADGVGEIAWTPIMRALGDGIVGSRFAQWVVLGAPPDLRADVVAAGLAAVVDTHDVLRLRVVDDRAGRRLAVGERGSVDTAGLVTRLECGGRPPDEVVERAVREAVGRLDPVAGVMAQAVWVDAGPARTGRLVVVVHHLAVDGMSWRILVPDLRLACEAVAEGRDPVLEPVWGSFRRWAALLEESALSRERVGELHTWRTIVDQEDRPVGRRRLSAGDAAGGVRSRSWVMSGDEASLLVGKVPVAFHCGVHEVLLAGLAGAVARWHGDDGVLVDVEGHGRHPAEGMDLSRTVGWFTSMHPVRLDVAGIELAAVPAGGRAAGQLLKAVKEQSRAAPGDGLGYGLLRHLNPETGPVLAALPSPQIGFNYMGRFVTVDQGGARPWQPVGGIGGSLDPGMGLPHALEVNAIVHDRLAGPELVLTVDWRDDLLEETDIERLCQVWLDMLSGLSRQAEDPSAGGHTASDFALLDLDQDEIEGFEAIAAELSGGQTS

SEQ ID NO: 27 - protein, 1063 aa, Nonomuria
MNTPSTPAGSALEEVWPLSPMQEGILYHAALDEAPDLYLIQQSQIIEGPLDTERFRLAWESLLNRHAALRACFHRRKSGESVQLIPRKVPLPWSERDLSGLSEEDALAEASVIAEKERATRFDPAKPPLLRQVLIRFGPDKHCLVTTSHHLVMDGWSRAILESELLELYAAGGAEPGLRPAGSYRDYLAWLERQDKEAARAAWRAELAGADRSTLGIPEASRKTQGQRVREVLGYAPDFTSALVDFARRHGLTLNTLVQGAWALVLARLTRRRDVVFGAVVSGRPAEVPGVEQAVGLFINTVPVRVRLDGGQPVIQLLTELQERQSTLISHQHLGLQEIQKLSGVSFDTVVSFENYVDPGAGPGSDRELRLRLKEFHQSAPYALLLGIMPGESLQTDVEYRPELLDARVAKEALHGLARVLERMIAEPETAVGRLDVVGDAGRELVVERWNETGDAIGAPSAVDLFRRQVARAPAATAVTAGDLAWSYAELDERSGRLARALTERGVRRGDRVGVVLGRSAEVLAAWLGVWKAGAAFVPVDPDYPADRVAFMLADSAVAMVVCQEATSGVVPPGYQQLLVNDADDGEAALVPIGADDLAYVMYTSGSTGTPKGVAIPHGGVAALAGDPGWGVGPGDAVLMHAPHTFDASLYDVWVPLVSGARVMITEPGVVDAERLAGHVADGLTAVNFTAGHFRALAQESPESFSGLREVAAGGDVVPLDVVERVRRACPRLRVWHTYGPTETTLCATWKAIEPGDEVGPVLPIGRALPGRRLYVLDAFLRPLPPGIAGDLYLAGAGVAHGYLGRASLTAERFVADPFVAGERMYRTGDLAYWTGEGELVFAGRDDDQVKIRGYRVEPGEVEAVLAGQPGVDQAVVVAREGRLLGYVVSGGGVDPVRLREGVARVLPEYMVPAAVVVLGAVPVTANGKVDREALPDPGFGGRVSGREPRTEVERALCGLFAEVLGLPGVTAVGPDDSFFELGGDSIHSVKLAARATRAGMPFTVVEVFEHKTPAGLATIVDVGGEPAAGPADPPSDSDLLGLAQDEIAEFEAEFDDERHSLR

SEQ ID NO: 28 - protein, 277 aa, Nonomuria
MISKAMHGPIRPARADTLLASVGERGILCDFYDENASEIFRDLEADAGGTEEAHGFAALVRPESGAILELGAGTGRLTIPLLELGWEVTALELSTAMLTTLRTRLADAPADLRDRCTLVHADMTAFKLGERFGTAILSPSTIDLLDDADRPGLYSSVREHLRPGGRFLLGMANPDASGRQEPLERTQEFTGRSGRRYVLHAKVYPSEEIRDVTIHPADESADPFVICVNRFRVITPDQIARELEQAGFDVVARTPLPGVRNHELVLEAQWGSVEDAH

SEQ ID NO: 29 - protein, 531 aa, Nonomuria
MSEELLFLRPDTIIEPLANRFYASMYATAPVTAAMNLAFRNLPMLESYLASPEWHFAAARDPKFRGGFFVNIEEQRKNEVEALLAAIRRDSADVLRFAEAIAEAEKIIREEATGYDLRPLYPKLPPELSGLVEIAYDTGNAASLHFLEPLIYKSKAYAEDCQSVQLSVETGIERPFVMSTPRLPSPDVLELNIPFRHPGLEELFLSRIRPTTLAALREALELGDAEAARLADLLVPEPSLASDRHVAAGARIRYWGHACLLMQTPDVAIMTDPFISADTDATGRYTYNDLPDRLDYVLITHGHSDHLVPETLLQLRGRVGTFVVPRTSRGNLCDPSLALYLRSFGLPAIEVDDFDEIEFPGGKIVSTPFFGEHADLDIRAKSTYWINLGGKSIWVGADSSGLDPVLYRHIRRHLGAVNIAFLGMECDGAPLNWQYQPFITKALPKKMSDSRKMSGSNAEQAGAIVTELGAEEAYIYAMGEESWLGHVMATSYNEDSYQLQQIAEFEAWCSRKGVKAAHLLDQHEWHWSSSR

SEQ ID NO: 30 - protein, 523 aa, Nonomuria
MTGGTGADAASAGASSTRPELRGERCLPPAGPVKVTPDDPRYLNLKLRGANSRFNGEPDYIHLVGSTQQVADAVEETVRTGKRVAVRSGGHCFEDFVDNPDVKVIIDMSLLTEIAYDPSMNAFLIEPGNTLSEVYEKLYLGWNVTIPGGVCGGVGVGGHICGGGYGPLSRQFGSVVDYLYAVEVVVVNKQGKARVIVATRERDDPHHDLWWAHTGGGGGNFGVVTKYWMRVPEDVGRNPERLLPKPPATLLTSTVTFDWAGMTEAAFSRLLRNHGEWYERNSGPDSPYTGLWSQLMIGNEVPGMGESGFMMPIQVDATRPDARRLLDAHIEAVIDGVPPAEVPEPIEQRWLASTPGRGGRGPASKTKAGYLRKRLTDRQIQAVYENMTHMDGIDYGAVWLIGYGGKVNTVDPAATALPQRDAILKVNYITGWANPGNEAKHLTWVRKLYADVYAETGGVPVPNDVSDGAYINYPDSDLADPGLNTSGVPWHDLYYKGNHPRLRKVKAAYDPRNHFHHALSIRP

SEQ ID NO: 31 - protein, 141 aa, Nonomuria
MTSTSGRHLYHRQVRFSDIDAHGHVNNVRFLEYLEDAWIALYLDNAGPPQEDRDGLPAVGFAVVRHEIFYRRPLRFRHGSVRVESWVTKVNRVTCEMAAQICSDGEVFVEARSMIMGFDTHTAKPRRLTLHERTFLKRYLR

SEQ ID NO: 32 - protein, 372 aa, Nonomuria
MGVDVSMTTSIASAEDLSVLTGLSEITTFAGVGTAVSATSYSQAELLEILDIRDPRIRSLFLNSAIERRFLALPPQGRDGERVAEPQGDLLDKHKKLAVDMGCRALESCLKSAGATLSDVRHLCCVTSTGFLTPGLSALIIRELGLDPHCSRADIVGMGCNAGLNALNLVAGWSAAHPGELAVVLCSEACSAAYALDGTMRTAVVNSLFGDGSAALAVVSGDGRAAGPRVLKFASYVITDAIEAMRYDWDRDQDRFSFFLDPQIPYVVGAHAEIVVDKLLSGTGLRRSDIGHWLVHSGGKKVIDAIVVNLGLSRHDVRHTTAVLRDYGNLSSGSFLFSYERLAGEGVTRPGDYGVLMTMGPGSTIETALIQW

SEQ ID NO: 33 - protein, 213 aa, Nonomuria
MNGELELSLDGTQALTASVEELNGLCDRAEDHRAPGPVIVHVTGVPRLGWSKGLTVGLVSKWERVVRRFERLGRLTVAVASGDCAGPSLDLLLAADVRIAAPATRLLPSWAGGAAWPGMAVYRLTQQAGTGGIRRAVLLGAPIDADRALALNLIDEVSADPAASLAGLAGAGDGAELAIRRQLMFEASSTTFEDALGAHLAAVDRALRRETLS

SEQ ID NO: 34 - protein, 434 aa, Nonomuria
MTTDWPALPPRAPLALWTLTAEAQRVDDLLAGLPEPPARTSAQRDAAASALDKVRRMRADYMEAHAEEIYGELTSGRTRHLRIDELVRAAARAYPGLVPTDEQMAAERARPQAEKEGREIDQGIFLRGVLRAPKAGPHLLDAMLRPTPRALELLPEFIESGEVRMEAVLLRRRDGVAYLTLCRDDCLNAEDAQQVDDMETAVDLALLDPQVRVGLLRGGEMSHPRYRGRRVFCAGVNLKKLSSGDISLVDFLLRRELGYIHKIVRGVYTDGSWHSKLTDKPWMAVVDSFAIGGGAQLLLVFDQVLAASDSYISLPAATEGIIPGVANYRLTRFTGPRAARQMILGGRRIRADEPDARLMIDEVVPPEEMDAAIDRALARLDGDAVPANRRMLNLAEEPPEAFGRYLAEFALQQALRIYGRDVIGKVGRFAAGSA

SEQ ID NO: 35 - protein, 265 aa, Nonomuria
MSEPRVRYEKKEHVAHVTMNRPHVLNAMDRRMHEELAEIWDDVEADDDVRTVVLTGAGTRAFSVGQDLKERALLDEAGTQASTFGSRGQAGHPRLTDRFTLSKPVVARVHGYALGGGFELVLACDLVIASEEAVFGLPEVRLGLIPGAGGVFRLPRQLPQKVAMGHLLTGRRMDAATAFRYGLVNEVVPLDELDRCVAGWTDDLVRAAPLSVRAIKEAAMRSLDIPLEEAFTTSYPWEERRRRSGDAIEGVRAFVEKRDPVWTSR

SEQ ID NO: 36 - protein, 428 aa, Nonomuria
MIPPHTLLVFFVQAAALLLLALLLGRLAVRLGLAAVVGELCAGVILGPSVLGQVAPGAEQWLFPSPSSHMLDAVGQLGVLLLIGLTGAHLDLRLIRRQGATAVRVSAFGLVVPMALGIGAGLLLPAEFRGTGGSAVFALFLGVTMCVSSIPVIAKTLMDMNLLHRNVGQLTLTAGMIDDAFGWVLLSVVTAMATAGAGAGTVVLSIASLLGVIVFSVVIGRPAVRVALRTTEDQGVIAGQVVVLVLAAAAGTHALGLEPIFGAFVAGLLVSTAMPNPVRLAPLRTVTLGVLAPLYFATMGLRVDLTALARPEVLAVGLLVLALAIIGKFLGAFLGAWTSRLSRWEALALGAGMNARGVIQMIVATVGLRLGVITDEIFTIIIVVAVITSLLAPPLLRLAMTRIEATAEEEARLLAYGLRPGEAREDVR

SEQ ID NO: 37 - protein, 251 aa, Nonomuria
MSTWFRCFDRRPLATMRLICFPHAGGSAVFYRNWHRLAAPEIEVHAVQYPGRADRLHEPLVGDAHRLAESVGRELRPLLDRPVALFGHSMGSLIAYETARLLTGSGIPPAHLFVSGGVAAHDRGRLAHRVAPASEEALIDRLRLLGGTDAEALASAEFRAFALPYVRNDFQLVQSYRHTPGPPLTVPITAFTGADDPVVRLDAVARWAELTAREFSCHVLPGGHFFLGHEQAALWAHLHARLGIATPAHCG

SEQ ID NO: 38 - protein, 428 aa, Nonomuria
MDSHVLAHQLSKETLHGSLMDPAIESMNLLNEIAGNYPDAISMAAGRPYEEFFDVGLIHDYLEAYRDHLRNDRRMDDAGISRMLFQYGTTKGIISDLVARHLAEDENIEADPASVVITVGFQEAMFLVLRALRANERDVLLAPTPTYVGLTGAALLTDTPVWPVQSTDNGIDLDHLEHQLKRAQDQGARVRACYVTPNFANPTGTSMDLPARHRLLEVAAAHGILILEDNAYGLLGQDRLPTLKSLDHAATVVYLGSFAKTGMPGARVGYVVADQHVAGGGSLADELAKLKGMLTVNTSPIAQAVIAGKLLRHDFSLARANARETAIYQRNLHLTLDELTRRLGAVPGVTWNAPTGGFFITVTVPFVVDDELLEHAARDHGVLFTPMHHFYGGKDGFNQLRLSISLLNPQLIEEGVSRLAGLVTACLP

SEQ ID NO: 39 - DNA, 18 bp, Artificial
  PCR primer
atgcgcgtgttgatctcg

SEQ ID NO: 40 - DNA, 18 bp, Artificial
  PCR primer
cggctgaccgcggcgaac

SEQ ID NO: 41 - DNA, 20 bp, Artificial
  PCR primer
cgtgggggtggatgtatcga

SEQ ID NO: 42 - DNA, 17 bp, Artificial
  PCR primer
tcaccattggatcagcg

SEQ ID NO: 43 - DNA, 18 bp, Artificial
  PCR primer
tcaggagacgaaccccgc

SEQ ID NO: 44 - DNA, 18 bp, Artificial
  PRC primer
gtgcacgaaagtcccgtc

SEQ ID NO: 45 - DNA, 18 bp, Artificial
  PCR primer
atggactcccacgttctc

SEQ ID NO: 46 - DNA, 18 bp, Artificial
  PCR primer
tcaggggagacatgcggt

SEQ ID NO: 47 - DNA, 29 bp, Artificial
  PCR primer
ttttgaattctcaggcgatccgtccgtct

SEQ ID NO: 48 - DNA, 31 bp, Artificial
  PCR primer
tttttctagagcccggacacccgggggctga

SEQ ID NO: 49 - DNA, 31 bp, Artificial
  PCR primer
tttttctagaagtcatggtgatgtgcgacat

SEQ ID NO: 50 - DNA, 30 bp, Artificial
  PCR primer
ttttaagcttatgttgcaggacgccgaccg

(19) United States
(12) Patent Application Publication
(10) Pub. No.: US 2008/0145892 A1
(43) Pub. Date: Jun. 19, 2008

(54) Genes and Proteins For the Biosynthesis of the Glycopeptide Antibiotic A40926

(75) Inventors: Stefano Donadio, Malnate (IT); Margherita Sosio, Solaro (IT); Fabrizio Beltrametti, Seveso (IT)

Correspondence Address: PFIZER INC., PATENT DEPARTMENT, MS8260-1611, EASTERN POINT ROAD, GROTON, CT, 06340, US

(73) Assignee: Pfizer, King of Prussia, PA (US)

(21) Appl. No.: 10/532,567

(22) PCT Filed: Oct. 15, 2003
(86) PCT No.: PCT/EP2003/011398

§ 371 Date: Apr. 22, 2005

(30) Foreign Application Priority Data
Oct. 23, 2002 (EP) 02023597.4

(51) Int. Cl. C12P 21/02; C07H 21/04; C12N 1/00; C07K 14/37; C12N 15/09

(52) U.S. Cl. 435/69.1; 536/23.74; 435/320.1; 435/243; 530/371

(57) ABSTRACT

The present invention relates to the field of antibiotics, and more specifically to the isolation of nucleic acid molecules that code for the biosynthetic pathway of the glycopeptide antibiotic A40926. Disclosed are the functions of the gene products involved in A40926 production. The present invention provides biosynthetic genes that code for A40926 production, the encoded polypeptides, the recombinant vectors comprising the nucleic acid sequences that encode said polypeptides, the host cells transformed with said vectors and methods for producing glycopeptide antibiotics using said transformed host cells, including methods for producing A40926, a precursor thereof, a derivative thereof or a modified glycopeptide different from A 40926 or a precursor thereof.

### BACKGROUND OF THE INVENTION

Actinomycetes are well known for their ability to produce structurally diverse and biologically active secondary metabolites, many of which have found commercial application (e.g. antibiotics). Important metabolites are not only produced by Streptomyces spp. (studied in most detail) but also by lesser known genera of actinomycetes: e.g. rifamycins, teicoplanin and erythromycin are currently produced industrially by Amycolatopsis, Actinoplanes and Saccharopolyspora species, respectively. The genetic elements governing the biosynthesis of secondary metabolites are organized in gene clusters, which contain all the genes required for synthesis of the metabolites, regulation and resistance.

Many different secondary metabolites share a common biosynthetic route, where similar enzymes intervene. This has been thoroughly documented for polyketides (Katz and McDaniel 1999), non-ribosomally synthesized peptides (Marahiel 1997) and deoxysugars (Rodriguez et al. 2000). However, despite this similarity, the organization of the gene cluster involved in the synthesis of a particular secondary metabolite in a given microorganism cannot be defined a priori. In fact, the synthesis of very similar secondary metabolites may be governed by differently organized clusters, especially when the corresponding producer strains do not belong to the same genus. Example of this sort can be found among the macrolide antibiotics (Katz and McDaniel 1999). Furthermore, the identification of a desired cluster within a producer strain is complicated in actinomycetes by the occurrence of multiple clusters specifying enzymes for the same pathway. This has been shown for polyketides (e.g. Ruan et al. 1997) and peptides (e.g. Sosio et al. 2000a), and confirmed by genome sequencing (Omura et al. 2001; Bentley et al. 2002). Consequently, one cannot know a priori the organization, nucleotide sequence, or extent of identity of a new cluster as compared to those already known.

Glycopeptides, also known as dalbaheptides because of their mechanism of action (Parenti and Cavalleri 1999), are an important class of antibiotics, interfering with cross-linking of the bacterial cell wall, with vancomycin and teicoplanin currently in clinical use. They are often last choice antibiotics in treating life-threatening infections. On the other hand, the emergence of resistance to glycopeptides among enterococci and the fear that this high-level resistance may eventually become widespread in methicillin-resistant Staphylococcus aureus has prompted the search for second-generation drugs of this class. Promising results have been obtained with the development of semi-synthetic derivatives with improved activity, expanded antibacterial spectrum or better pharmacokinetics (Malabarba and Ciabatti 2001).

Therefore, there exists the potential and the utility to obtain improved glycopeptides by manipulation of occurring natural compounds. However, glycopeptides are structurally complex molecules and their accessibility to chemistry is limited to a few positions in the molecule. For example, while the sugars can be easily removed chemically from a glycopeptide, generating the corresponding aglycone, the regioselective attachment of a different sugar to a particular position by chemical means is extremely difficult. It has been shown that the extent of chlorination in glycopeptides influences antibiotic activity. Similarly, the chemical dechlorination of aromatic rings in glycopeptides can be easily achieved, while the selected halogenation of desired rings in the structure is relatively complex. As a final example, glycopeptides of the teicoplanin family contain an acyl chain linked to the glucosamine attached to the arylamino acid at position 4, while compounds of the vancomycin class do not. Acylation and deacylation of glycopeptides has been reported either chemically or by biotransformation (Lancini and Cavalleri 1997), but it usually results in overall low yields. In light of the above, it would be desirable to have genes and enzymes useful for redirecting these steps in glycopeptide formation, in order to obtain derivatives that are hard or impossible to make by chemical means. This is particularly relevant, since it has been shown that the extent of chlorination influences the biological activity of glycopeptides, as well as that improved derivatives can be obtained by altering the glycosylation or acylation pattern of glycopeptides (Malabarba and Ciabatti 2001). One of the major limitations for chemistry is to change the type or order of amino acids present in the peptide backbone. Chemically, it has been shown to be possible to intervene only on amino acids 1 and 3 with relatively low yield (Malabarba et al. 1997). General methods for the design of novel glycopeptide derivatives directly by fermentation processes with precisely engineered strains would thus be highly desirable.

An attractive alternative would be to generate improved antibiotics by engineering of biosynthetic processes for naturally occurring glycopeptides. Examples of this sort have been reported. Indeed, it has been possible to selectively glycosylate glycopeptide aglycons both in vitro and in vivo after the expression of glycosyltransferases from the vancomycin and chloroeremomycin gene clusters (Solenberg et al. 1997; Loosey et al. 2001). However, none of the enzymes described so far is able to attach a glucosamine residue at desired positions. Similarly, inactivation of selected genes in the balhimycin producer A. mediterranei has led to the obtainment of balhimycin derivatives (Pelzer et al. 1999). However, no such experiments have been described for strains producing glycopeptides of the teicoplanin family.

The antibiotic A40926 belongs to the teicoplanin family of glycopeptides (Parenti and Cavalleri 1989). It consists of a complex of closely related molecules, whose core structure can be reconducted to a heptapeptide skeleton with a rigid scaffold determined by ether bonds between amino acids 1-3, 2-4 and 4-6, and a C—C bond between amino acids 5-7. In addition two sugar residues and two chlorine atoms are present on the molecule. The structure of the components of A40926 complex is represented by the formula shown below, wherein R represents [C₉-C₁₂] alkyl with the factors A₁(R=n-decyl), factor B₀ (R=9-methyldecyl) and factor B₁ (R=n-undecyl) being the main components.

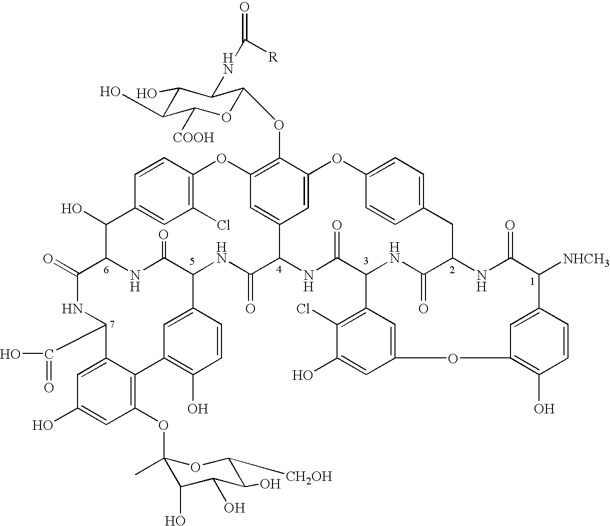

The producer strain, formerly known as Actinomadura sp. ATCC39727, has been recently reclassified as Nonomuria sp. ATCC39727 (Zhang et al. 1998). Besides showing an intrinsic antibacterial activity, A40926 is also the precursor of the semi-synthetic glycopeptide dalbavancin (formerly known as BI397 or MDL 62397; Malabarba and Ciabatti 2001). Therefore, additional tools for manipulating the structure of A40926 and for increasing its yield would be highly desirable. However, there are no examples of clusters described from other members of the genus Nonomruia. Therefore, the genes required for and regulating the formation of A40926 in Nonomuria can also be useful in optimizing the production process.

Recently, gene clusters involved in the formation of the glycopeptides chloroeremomycin (van Wageningen et al. 1998), balhimycin (Pelzer et al. 1999), complestatin (Chiu et al. 2001) and A47934 (Pootoolal et al. 2002) have been described. These clusters, designated cep, bad, com and sta, respectively, were obtained from Amycolatopsis orientalis, Amycolatopsis mediterranei, Streptomyces lavendulae and Streptomyces toyocaensis, respectively. These clusters have provided several genes useful for manipulating glycopeptide pathways. However, certain steps cannot be performed with the described clusters. For example, the available gene clusters do not encode functions capable of changing the oxidation state of sugars, of attaching a fatty acid chain, or of providing a chlorine atom at the aromatic moiety of amino acid 3. All these functions are also described in the present invention.

The design of industrial processes for antibiotic production has been relatively successful, resulting in large size fermentations with antibiotic titers reaching levels of several grams per liter. This has been achieved largely by following empirical, trial and error approaches, and lacks a rational basis. Development of new processes and improvement of current technology thus remains time consuming and may result in bacterial cultures that are unstable, perform inconsistently and accumulate unwanted by-products. In recent years, rational methods have been applied successfully to increase the level of antibiotic produced by Streptomyces spp., which have often involved the manipulation of key regulatory elements present within the gene cluster of interest or the overexpression of rate-limiting steps in the pathway. Therefore, the genes encoding such cluster-associated regulators or limiting steps in the synthesis can be effective tools for yield improvement. However, the cluster-associated regulators so far identified in actinomycetes belong to several different protein families (Chater and Bibb 1997). Even within one family, there is considerable variation in sequence identity. Therefore, the existence, nature, number and sequence of cluster-associated regulators cannot be predicted by comparison to other cluster, even those specifying a related antibiotic. As an example, the tylosin gene cluster encodes four distinct regulators, while none has been found in the cluster specifying the related macrolide antibiotic erythromycin (Bate et al. 1999). Similarly, the nature and reason for a rate-limiting step in a biosynthetic pathway cannot be established a priori.

### SUMMARY OF THE INVENTION

The present invention provides a set of isolated polynucleotide molecules required for the biosynthesis of the glycopeptide A40926 in microorganisms. In one form of the invention, polynucleotide molecules are selected from the contiguous DNA sequence (SEQ ID NO: 1), which represents the dbv gene cluster as isolated from Nonomuria sp. ATCC39727 and consists of 37 ORFs encoding the polypeptides required for A40926 formation. The amino acid sequences of the polypeptide encoded by said 37 ORFs are provided in SEQ ID NOS: 2 to 38.

The present invention provides an isolated nucleic acid comprising a nucleotide sequence selected from a group consisting of:
a) the dbv gene cluster encoding the polypeptides required for the synthesis of A40926 (SEQ ID NO: 1);
b) a nucleotide sequence encoding the same polypeptides encoded by the dbv gene cluster (SEQ ID NO. 1), other than the nucleotide sequence of the dbv gene cluster itself;
c) any nucleotide sequence of dbv ORFs 1 to 37, encoding the polypeptides of SEQ ID NOS: 2 to 38;
d) a nucleotide sequence encoding the same polypeptide encoded by any of dbv ORFs 1 to 37 (SEQ ID NOS: 2 to 38), other than the nucleotide sequence of said ORF.

A further object of this invention is to provide an isolated nucleic acid comprising a nucleotide sequence selected from the group consisting of:
e) a nucleotide sequence of any of dbv ORFs 3 to 4, 6 to 10, 18 to 20, 22 to 23, 29 to 30, and 36, encoding the polypeptides specified in SEQ ID NOS: 4 to 5, 7 to 11, 19 to 21, 23 to 24, 30 to 31, and 37;
f) a nucleotide sequence encoding the same polypeptide encoded by any of dbv ORFs 3 to 4, 6 to 10, 18 to 20, 22 to 23, 29 to 30, and 36 (SEQ ID NOS: 4 to 5, 7 to 11, 19 to 21, 23 to 24, 30 to 31, and 37) other than the nucleotide sequence of said dbv ORF;
g) a nucleotide sequence encoding a polypeptide that is at least 80%, preferably 86%; more preferably 90%, most preferably 95% or more, identical in amino acid sequence to a polypeptide encoded by any of dbv ORFs 3, 6 to 9, 18 to 20, 22 to 23, 29 to 30, and 36 (SEQ ID NOS: 4, 7 to 10, 19 to 21, 23 to 24, 30 to 31, and 37);
h) a nucleotide sequence encoding a polypeptide that is at least 87%, preferably 90%; more preferably 95% or more, identical in amino acid sequence to a polypeptide encoded by any of dbv ORFs 4 and 10 (SEQ ID NOS: 5 and 11).
   In one embodiment the isolated nucleic acids of this invention comprise combinations of ORFs selected from ORFs 1 to 37 (SEQ ID NOS: 2 to 38), which encode polypeptides required for the synthesis of 4-hydroxyphenylglycine (HPG) residues of A40926. In another embodiment, the nucleic acid comprises combinations of ORFs selected from ORFs 1 to 37 (SEQ ID NOS: 2 to 38), which encode the polypeptides required for the synthesis of 3,5-dihydroxyphenylglycine (DPG) residues of A40926. In yet another embodiment, the nucleic acid comprises combinations of ORFs selected from ORFs 1 to 37 (SEQ ID NOS: 2 to 38), which encode the polypeptides required for the synthesis of the heptapeptide skeleton of A40926. According to another embodiment, in a nucleic acid of this invention, combinations of ORFs selected from ORFs 1 to 37 (SEQ ID NOS: 2 to 38) are provided which encode a polypeptide required for the chlorination of the aromatic residues of amino acids 3 and 6 of A40926. In yet another embodiment, nucleic acid comprising combinations of ORFs selected from ORFs 1 to 37 (SEQ ID NOS: 2 to 38) are provided, which encode a polypeptide required for the β-hydroxylation of the tyrosine residue of aminoacid 6 of A40926. In yet another embodiment, nucleic acid comprising combinations of ORFs selected from ORFs 1 to 37 (SEQ ID NOS: 2 to 38) are provided, which encode polypeptides required for the cross-linking of the aromatic residues of amino acids at positions 2 and 4, 4 and 6, 1 and 3, and 5 and 7 of A40926. According to another embodiment, in the nucleic acid of this invention, combinations of ORFs selected from ORFs 1 to 37 (SEQ ID NOS: 2 to 38) are provided which encode the polypeptides required for the addition and formation of the N-acylglucuronamine residue. In yet another embodiment, nucleic acids are provided which comprise combinations of ORFs selected from ORFs 1 to 37 (SEQ ID NOS: 2 to 38), encoding a polypeptide required for the attachment of the mannosyl residue. In yet another embodiment, nucleic acids are provided which comprise combinations of ORFs selected from ORFs 1 to 37 (SEQ ID NOS: 2 to 38), encoding a polypeptide required for the N-methylation of A40926. According to yet another embodiment, nucleic acids are provided which comprise combinations of ORFs selected from ORFs 1 to 37 (SEQ ID NOS: 2 to 38), encoding polypeptides required for the export of and resistance to A40926. In yet another embodiment, nucleic acids are provided which comprise combinations of ORFs selected from ORFs 1 to 37 (SEQ ID NOS: 2 to 38), encoding polypeptides required for regulating the expression of the dbv gene cluster. In yet another embodiment, nucleic acids are provided which comprise one or more DNA segments selected from SEQ ID NO: 1, enhancing the expression level of an ORF selected from ORFs 1 through 37 (SEQ ID NOS: 2 to 38).

Those skilled in the art understand that the present invention, having provided the nucleotide sequences encoding polypeptides of the A40926 biosynthetic pathway, also provides nucleotides encoding fragments derived from such polypeptides. In addition, those skilled in the art understand that, since the genetic code is degenerate, the same polypeptides specified in SEQ ID NOS: 2 to 38 can be encoded by natural or artificial variants of ORFs 1 to 37, i.e. by nucleotide sequences other than the genomic nucleotide sequences specified by ORFs 1 to 37 but which encode the same polypeptides. Furthermore, it is also understood that naturally occurring or artificially manufactured variants can occur of the polypeptides specified in SEQ ID NOS: 2 to 38, said variants having the same function(s) as the above mentioned original polypeptides but containing addition, deletion or substitution of amino acid not essential for folding or catalytic function, or conservative substitution of essential amino acids.

Those skilled in the art understand also that, having provided the nucleotide sequence of the entire cluster required for A40926 biosynthesis, the present invention also provides nucleotide sequences required for the expression of the genes present in said cluster. Such regulatory sequences include but are not limited to promoter and enhancer sequences, antisense sequences, transcription terminator and antiterminator sequences. These sequences are useful for regulating the expression of the genes present in the dbv gene cluster. Cells carrying said nucleotide sequences, alone or fused to other nucleotide sequences, fall also within the scope of the present invention.

In one aspect, the present invention provides isolated nucleic acids comprising nucleotide sequences encoding the ORF9 polypeptide (SEQ ID NO: 10), or naturally occurring variants or derivatives of said polypeptide, useful for the attachment of an N-acyl-glucosamine residue to the core structure of a glycopeptide antibiotic precursor. In another aspect, the present invention provides nucleic acids comprising nucleotide sequences encoding the ORF23 polypeptide (SEQ ID NO: 24), or naturally occurring variants or derivatives of said polypeptide, useful for the attachment of fatty acid residues to the core structure of a glycopeptide antibiotic precursor. In yet another aspect, the present invention provides a nucleic acid comprising nucleotide sequences encoding the ORF29 polypeptide (SEQ ID NO: 30), or naturally occurring variants or derivatives of said polypeptide, useful for the oxidation of sugar moieties attached to a glycopeptide antibiotic precursor. In another aspect, the present invention provides nucleic acids comprising nucleotide sequences encoding the ORF10 polypeptide (SEQ ID NO: 11), or naturally occurring variants or derivatives of said polypeptide, useful for the chlorination of b-hydroxytyrosine and DPG residues in a core glycopeptide antibiotic precursor. In another aspect, the present invention provides nucleic acids comprising nucleotide sequences encoding the ORF20 polypeptide (SEQ ID NO: 21), or naturally occurring variants or derivatives of said polypeptide, useful for the attachment of mannosyl residues to the core structure of a glycopeptide antibiotic precursor.

In another aspect, the present invention provides nucleic acids comprising nucleotide sequences encoding the polypeptides encoded by ORFs 7, 18, 19, 24 and 35 (SEQ ID NOS: 8, 19, 20, 25 and 36), or naturally or artificially occurring variants or derivatives of said polypeptides, useful for export out of the cells of a glycopeptide antibiotic or a glycopeptide antibiotic precursor and conferring resistance. In another aspect, the present invention provides nucleic acids comprising nucleotide sequences encoding the ORF7 polypeptide (SEQ ID NO: 8), or naturally or artificially occurring variants or derivatives of said polypeptide, useful for conferring resistance to the producing strain to a glycopeptide antibiotic or a glycopeptide antibiotic precursor. In another aspect, the present invention provides nucleic acids comprising nucleotide sequences encoding the ORFs 3, 4, 6, 22 and 36 polypeptide (SEQ ID NOS: 4, 5, 7, 23 and 37), or naturally or artificially occurring variants or derivatives of said polypeptides, useful for increasing the yield of a glycopeptide antibiotic precursor.

In one embodiment, the present invention provides a glycopeptide producing strain carrying extra copies of the nucleotide sequences specifying at least one ORF selected from any of ORFs 1 through 37 (SEQ ID NOS: 2 to 38). In one preferred embodiment, such glycopeptide producing strain is any strain belonging to the order Actinomycetales. In yet another preferred embodiment, such glycopeptide producing strain is a member of the genus Nonomuria. In one further aspect, the present invention provides a Nonomuria strain containing one or more variations in the nucleotide sequence specified in SEQ ID NO: 1, such variation resulting in an increased or decreased expression of one or more of ORFs 1 through 37 (SEQ ID NOS: 2 to 38).

In one preferred embodiment, the present invention provides nucleic acids comprising a nucleotide sequence specified by SEQ ID NO: 1, or a portion thereof, carried on one or more vectors, useful for the production of A40926, one or more of its precursors or a derivative thereof by another cell. In one preferred embodiment, said nucleotide sequence or portion thereof is carried on a single vector. In yet another preferred embodiment, such vector is a bacterial artificial chromosome. In yet another aspect, said bacterial artificial chromosome is an ESAC vector (as described in WO99/63674). In another preferred embodiment, the present invention provides a recombinant actinomycete strain other than Nonomuria sp. ATCC 39727 containing the gene cluster specified by SEQ ID NO: 1, said gene cluster being carried in an ESAC vector which is integrated into the chromosome of said recombinant actinomycete strain.

In one aspect, the present invention provides a method for increasing the production of A40926, said method comprising the following steps: (1) transforming with a recombinant DNA vector a microorganism that produces A40926 or a A40926 precursor by means of a biosynthetic pathway, said vector comprising a DNA sequence, chosen from any of ORFs 1 through 37 (SEQ ID NO: 2 through 38), that codes for an activity that is rate limiting in said pathway; (2) culturing said microorganism transformed with said vector under conditions suitable for cell growth, expression of said gene and production of said antibiotic or antibiotic precursor.

In another aspect, the present invention provides a method for producing derivatives of A40926, said method comprising the following steps: (1) cloning in a suitable vector a segment chosen from the nucleotide sequence defined by SEQ ID NO:1, said segment containing at least a portion of one of ORFs 1 through 37 (SEQ ID NO: 2 through 38), said ORF encoding a polypeptide that catalyzes a biosynthetic step that one wishes to bypass; (2) inactivating said ORF by removing or replacing one or more codons that specify for amino acids that are essential for the activity of said polypeptide; (3) transforming with said recombinant DNA vector a microorganism that produces A40926 or a A40926 precursor by means of a biosynthetic pathway; (4) screening the resulting transformants for those where said DNA sequence has been replaced by the mutated copy, thus creating a disrupted gene; and (5) culturing said mutant cells under conditions suitable for cell growth, expression of said pathway and production of said pathway analogue.

In yet another aspect, the present invention provides a method for producing novel glycopeptides, said method comprising the following steps: (1) transforming with a recombinant DNA vector a microorganism that produces a glycopeptide or a glycopeptide precursor different from A40926 or a precursor thereof by means of a biosynthetic pathway, said vector comprising one or more ORFs, chosen among ORFs 1 through 37 (SEQ ID NOS: 2 through 38), coding for the expression of one or more polypeptide(s) that modifies) said glycopeptide or glycopeptide precursor; (2) culturing said microorganism transformed with said vector under conditions suitable for cell growth, expression of said gene and production of said antibiotic or antibiotic precursor.

### Examples of Microorganisms that Produce a Glycopeptide or a Glycopeptide Precursor Suitable for Carrying Out this Method are Strains Belonging to the Genera Streptomyces, Amycolatopsis, Actinoplanes, Nonomuria and the Like.

In yet another aspect, the present invention provides a further method for producing novel glycopeptides, said method comprising the following steps: (1) transforming with a recombinant DNA vector a microorganism, said vector comprising one or more ORFs, chosen among ORFs 1 through 37 (SEQ ID NOS: 2 through 38), coding for one or more polypeptide(s) that modifies(y) a glycopeptide or glycopeptide precursor (active polypeptide(s)), and said microorganism being selected among those that do not produce glycopeptides or glycopeptide precursors and that can efficiently express the introduced ORF(s); (2) preparing a cell extract or cell fraction of said microorganism under conditions suitable for the presence of active polypeptide(s), said cell extract or cell fraction containing at least said active polypeptide(s); (3) adding a glycopeptide or glycopeptide precursor to said cell extract or cell fraction, and incubating said mixture under conditions where said active polypeptide(s) can modify said glycopeptide or glycopeptide precursor.

### Examples of Microorganisms Suitable for Carrying Out this Method are Strains Belonging to the Species Streptomyces lividans, Streptomyces coelicolor, Escherichia coli, Bacillus subtilis and the Like

A further aspect of this invention includes an isolated polypeptide comprising a polypeptide sequence involved in the biosynthetic pathway of A40926 selected from
a) an ORF polypeptide encoded by any of dbv ORFs 1 to 37 (SEQ ID NOS: 2 through 38) or a polypeptide which is, identical in amino acid sequence to a polypeptide encoded by any of dbv ORFs 1 to 37 (SEQ ID NOS: 2 through 38), preferably by any one of the dbv ORFs 3 to 4, 6 to 10, 18 to 20, 22 to 23, 29 to 30 (SEQ ID NOS: 4 to 5, 7 to 11, 19 to 21, 23 to 24, 30 to 31 and 37);
b) a polypeptide which is at least 80% preferably 86%, more preferably 90%, most preferably 95% or more, identical in amino acid sequence to a polypeptide encoded by any of dbv ORFs 3, 6 to 9, 18 to 20, 22 to 23, 29 to 30 and 36 (SEQ ID NOS: 4, 7 to 10, 19 to 21, 23 to 24, 30 to 31 and 37); and
c) a polypeptide which is at least 87%, preferably 90%, more preferably 95% or more, identical in amino acid sequence to a polypeptide encoded by any of the dbv ORFs 4 and 10 (SEQ ID NOS: 5 to 11).

### DEFINITIONS

The term “isolated nucleic acid” refers to a DNA molecule, either as genomic DNA or a complementary DNA (cDNA), which can be single or double stranded, of natural and synthetic origin. This term refers also to an RNA molecule, of natural or synthetic origin.

The term “nucleotide sequence” refers to full length or partial length sequences of ORFs and intergenic regions as disclosed herein. Any one of the nucleotide sequences of the invention as shown in the sequence listing is (a) a coding sequence, (b) an RNA molecule derived from transcription of (a), (c) a coding sequence which uses the degeneracy of the genetic code to encode an identical polypeptide, or (d) an intergenic region, containing promoters, enhancers, terminator and antiterminator sequences.

The terms “gene cluster”, “cluster” and “biosynthesis cluster” all designate a contiguous segment of a microorganism's genome that contains all the genes required for the synthesis of a secondary metabolite.

The term “dbv” refers to a genetic element responsible for A40926 biosynthesis in Nonomuria sp. ATCC39727.

The term “ORF” refers to a genomic nucleotide sequence that encodes one polypeptide. In the context of the present invention, the term ORF is synonymous with “gene”.

The term “ORF polypeptide” refers to a polypeptide encoded by an ORF.

The term “dbv ORF” refers to an ORF comprised within the dbv gene cluster.

The term “NRPS” refers to a non-ribosomal peptide synthetase which is a complex of enzymatic activities responsible for the incorporation of amino acids into an oligopeptide skeleton of a secondary metabolite. A functional NRPS is one that catalyzes the incorporation of one or more amino acid into an oligopeptide.

The term “NRPS module”, or “module”, refers to a segment of a NRPS that directs the activation, incorporation and possible modification of one amino acid into an oligopeptide.

The term “NRPS gene” refers to a gene that encodes an NRPS.

The term “secondary metabolite” refers to a bioactive substance produced by a microorganism through the expression of a set of genes specified by a gene cluster.

The term “production host” is a microorganism where the formation of a secondary metabolite is directed by a gene cluster derived from a donor organism.

The term “ESAC” identifies an “Escherichia coli-Streptomyces Artificial Chromosome”, i.e. a recombinant vector that carries and maintains large DNA inserts in an Escherichia coli host and that can be introduced and maintained in an actinomycete production host. Examples of ESACs are given in WO99/67374.

### BRIEF DESCRIPTION OF THE DRAWINGS

FIG. 1. Isolated DNA segments derived from the chromosome of Nonomuria sp. ATCC39727. The thick line denotes the segment described in SEQ ID NO: 1. The cosmids carrying said isolated DNA segments are designated 11A5, 7F3, 7E9, 1B1, 7A2, 11B9 and 7C7.

FIG. 2. Genetic organization of the dbv cluster. Each ORF is represented by an arrow, and numbered as in Table 1. The orientation is the same as in FIG. 1. Numbers on the scale bars indicate sequence coordinates (in kb).

### DETAILED DESCRIPTION OF THE INVENTION

A. The dbv Genes from Nonomuria

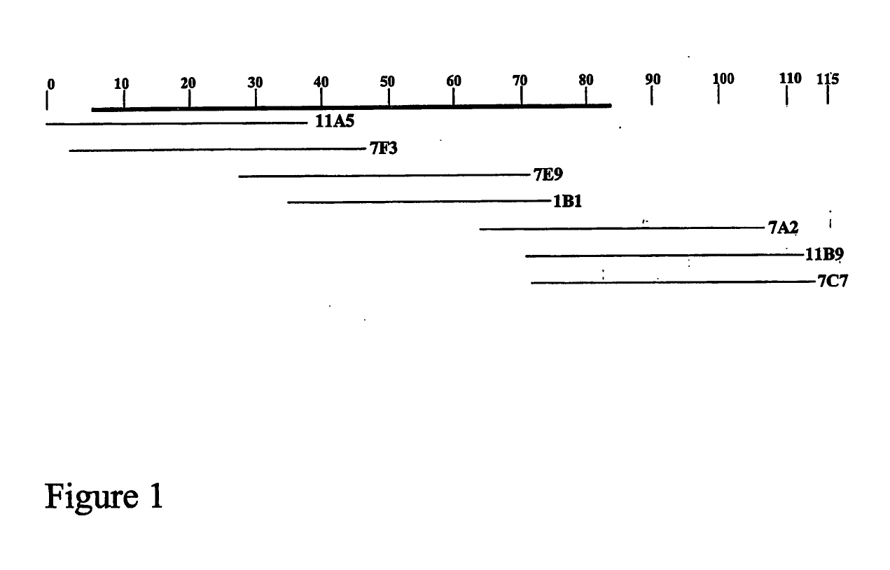

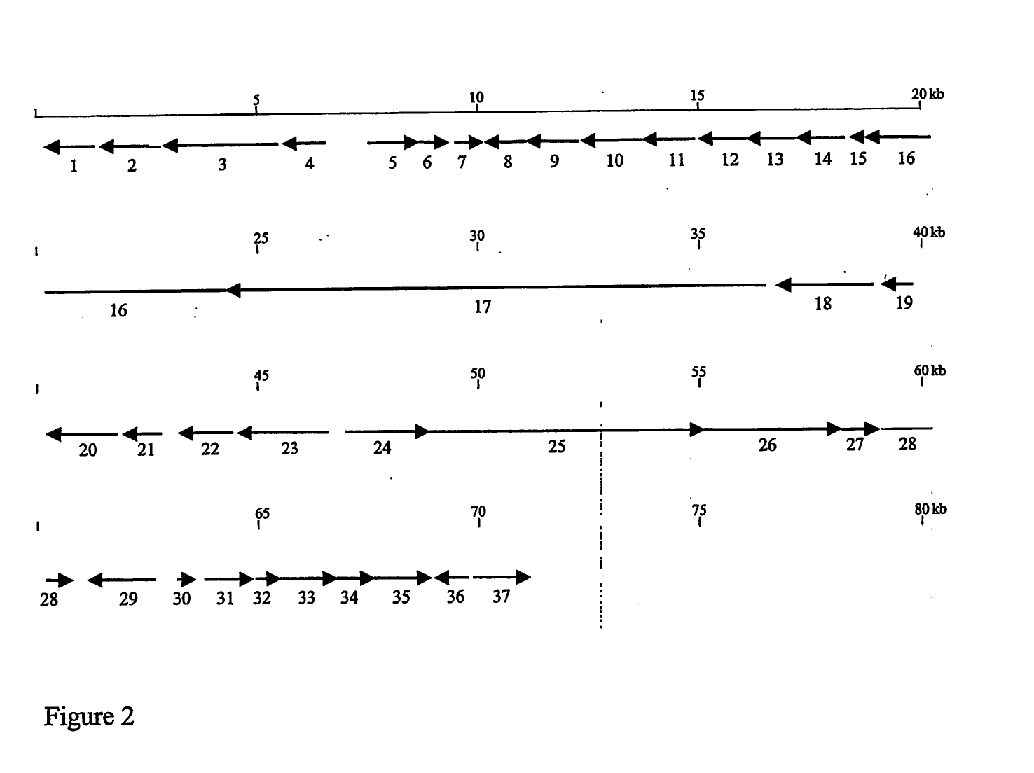

A40926 is a complex of closely related glycopeptide antibiotics produced by Nonomuria sp. ATCC39727. The present invention provides nucleic acid sequences and characterization of the gene cluster for the biosynthesis of A40926. The physical organization of the A40926 gene cluster, together with flanking DNA sequences, is reported in FIG. 1, which illustrates the physical map of a 90-kb genomic segment from the genome of Nonomuria sp. ATCC39727, together with a set of cosmids defining such segment. The genetic organization of the DNA segment governing A40926 biosynthesis, designated as the dbv cluster, is shown in FIG. 2 and its nucleotide sequence is reported as SEQ ID NO: 1.

The precise boundary of the cluster can be established by comparison with other glycopeptide clusters and from the functions of its gene products. Therefore, on the left end (FIG. 1) the dbv cluster is delimited by dbv ORF1, encoding the enzyme HmoS (SEQ ID No: 2), involved in the synthesis of HPG. On the right side, the dbv cluster is delimited by a remnant of an attL site, similar to the 3′-end of a tRNA gene, spanning nucleotides 71065 to 71138 of SEQ ID NO: 1. The dbv cluster spans approximately 71,100 base pairs and contains 37 ORFs, designated dbv ORF1 through dbv ORF37. The contiguous nucleotide sequence of SEQ ID NO: 1 (71138 base pairs) encodes the 37 deduced proteins listed in SEQ ID NOS: 2 to 38. ORF1 (SEQ ID NO: 2) represents 366 amino acids deduced from translating SEQ ID NO: 1 from nucleotides 1140 to 40 on the complementary strand. ORF2 (SEQ ID NO: 3) represents 356 amino acids deduced from translating SEQ ID NO: 1 from nucleotides 2329 to 1259 on the complementary strand. ORF3 (SEQ ID NO: 4) represents 867 amino acids deduced from translating SEQ ID NO: 1 from nucleotides 5161 to 2558 on the complementary strand. ORF4 (SEQ ID NO. 5) represents 321 amino acids deduced from translating SEQ ID NO: 1 from nucleotides 6231 to 5266 on the complementary strand. ORF5 (SEQ ID NO: 6) represents 369 amino acids deduced from translating SEQ ID NO: 1 from nucleotides 7183 to 8292. ORF6 (SEQ ID NO: 7) represents 217 amino acids deduced from translating SEQ ID NO: 1 from nucleotides 8320 to 8973. ORF7 (SEQ ID NO: 8) represents 196 amino acids deduced from translating SEQ ID NO: 1 from nucleotides 9069 to 9659. ORF8 (SEQ ID NO: 9) represents 319 amino acids deduced from translating SEQ ID NO: 1 from nucleotides 10667 to 9708 on the complementary strand. ORF9 (SEQ ID NO: 10) represents 408 amino acids deduced from translating SEQ ID NO: 1 from nucleotides 11896 to 10670 on the complementary strand. ORF10 (SEQ ID NO: 11) represents 489 amino acids deduced from translating SEQ ID NO: 1 from nucleotides 13419 to 11950 on the complementary strand. ORF11 (SEQ ID NO: 12) represents 420 amino acids deduced from translating SEQ ID NO: 1 from nucleotides 14741 to 13479 on the complementary strand. ORF12 (SEQ ID NO: 13) represents 398 amino acids deduced from translating SEQ ID NO: 1 from nucleotides 16019 to 14823 on the complementary strand. ORF13 (SEQ ID NO: 14) represents 384 amino acids deduced from translating SEQ ID NO: 1 from nucleotides 17163 to 16009 on the complementary strand. ORF14 (SEQ ID NO: 15) represents 393 amino acids deduced from translating SEQ ID NO: 1 from nucleotides 18366 to 17185 on the complementary strand. ORF15 (SEQ ID NO: 16) represents 69 amino acids deduced from translating SEQ ID NO: 1 from nucleotides 18671 to 18462 on the complementary strand. ORF16 (SEQ ID NO: 17) represents 1863 amino acids deduced from translating SEQ ID NO: 1 from nucleotides 24259 to 18668 on the complementary strand. ORF17 (SEQ ID NO: 18) represents 4083 amino acids deduced from translating SEQ ID NO: 1 from nucleotides 36529 to 24278 on the complementary strand. ORF18 (SEQ ID NO: 19) represents 753 amino acids deduced from translating SEQ ID NO: 1 from nucleotides 39021 to 36760 on the complementary strand. ORF19 (SEQ ID NO: 20) represents 232 amino acids deduced from translating SEQ ID NO: 1 from nucleotides 39851 to 39152 on the complementary strand. ORF20 (SEQ ID NO: 21) represents 535 amino acids deduced from translating SEQ ID NO: 1 from nucleotides 41732 to 40125 on the complementary strand. ORF21 (SEQ ID NO: 22) represents 270 amino acids deduced from translating SEQ ID NO: 1 from nucleotides 42584 to 41772 on the complementary strand. ORF22 (SEQ ID NO: 23) represents 420 amino acids deduced from translating SEQ ID NO: 1 from nucleotides 44130 to 42868 on the complementary strand. ORF23 (SEQ ID NO: 24) represents 709 amino acids deduced from translating SEQ ID NO: 1 from nucleotides 46355 to 44226 on the complementary strand. ORF24 (SEQ ID NO: 25) represents 648 amino acids deduced from translating SEQ ID NO: 1 from nucleotides 46632 to 48578. ORF25 (SEQ ID NO: 26) represents 2097 amino acids deduced from translating SEQ ID NO: 1 from nucleotides 48575 to 54868. ORF26 (SEQ ID NO: 27) represents 1063 amino acids deduced from translating SEQ ID NO: 1 from nucleotides 54865 to 58056. ORF27 (SEQ ID NO: 28) represents 277 amino acids deduced from translating SEQ ID NO: 1 from nucleotides 58152 to 58985. ORF28 (SEQ ID NO: 29) represents 531 amino acids deduced from translating SEQ ID NO: 1 from nucleotides 59046 to 60641. ORF29 (SEQ ID NO: 30) represents 523 amino acids deduced from translating SEQ ID NO: 1 from nucleotides 62445 to 60874 on the complementary strand. ORF30 (SEQ ID NO: 31) represents 141 amino acids deduced from translating SEQ ID NO: 1 from nucleotides 62887 to 63312. ORF31 (SEQ ID NO: 32) represents 372 amino acids deduced from translating SEQ ID NO: 1 from nucleotides 63469 to 64587. ORF32 (SEQ ID NO: 33) represents 213 amino acids deduced from translating SEQ ID NO: 1 from nucleotides 64599 to 65240. ORF33 (SEQ ID NO: 34) represents 434 amino acids deduced from translating SEQ ID NO: 1 from nucleotides 65237 to 66541. ORF34 (SEQ ID NO: 35) represents 265 amino acids deduced from translating SEQ ID NO: 1 from nucleotides 66538 to 67335. ORF35 (SEQ ID NO: 36) represents 428 amino acids deduced from translating SEQ ID NO: 1 from nucleotides 67332 to 68618. ORF36 (SEQ ID NO: 37) represents 251 amino acids deduced from translating SEQ ID NO: 1 from nucleotides 69423 to 68685 on the complementary strand. ORF37 (SEQ ID NO: 38) represents 428 amino acids deduced from translating SEQ ID NO: 1 from nucleotides 69608 to 70894.

The dbv cluster presents an organization that substantially differs from those of other glycopeptide clusters. A comparison among the five bal, cep, com, sta and dbv clusters is summarized in TABLE 1

TABLE 1

| dbv cluster | | | glycopeptide clusters^{a} | | | | | GeneBank^{b} | | | |
|---|---|---|---|---|---|---|---|---|---|---|---|
| dbv | size | | | | | | Best | | proba- | | |
| ORF | (Da) | Proposed function^{c} | bal | cep | com | sta | match^{d} | entry^{e} | bility^{f} | Source, function^{g} | CD^{h} |
| ORF1 | 38146 | p-hydroxymandelate | + | + | + | + | sta, 65% | | | | |
| | | oxidase | | | | | | | | | |
| ORF2 | 37922 | p-hydroxymandelate | + | + | + | + | sta, 65% | | | | |
| | | synthetase | | | | | | | | | |
| ORF3 | 93001 | Regulator | | | | | | T03225 | 9e−90 | S. hygroscopicus, | |
| | | | | | | | | | | positive regulator | |
| ORF4 | 35408 | Regulator | | + | + | + | cep, 81% | | | | |
| ORF5 | 38817 | prephenate | | + | + | + | cep, 82% | | | | |
| | | dehydrogenase | | | | | | | | | |
| ORF6 | 23902 | response regulator | | | * | * | com, 66% | Q03756 | 9e−84 | S. coelicolor, | |
| | | | | | | | | | | response regulator | |
| | | | | | | | | | | CutR | |
| ORF7 | 22157 | Carboxypeptidase | | | | | | S77033 | 8e−04 | Synechocystis sp., | VanY-type |
| | | | | | | | | | | unknown | carboxy- |
| | | | | | | | | | | | peptidase |
| ORF8 | 36364 | Unknown | | | | | | no | | | |
| | | | | | | | | matches | | | |
| ORF9 | 42916 | Glycosyltransferase | + | + | | | cep, 69% | | | | |
| ORF10 | 53813 | Halogenase | + | + | + | + | sta, 86% | | | | |
| ORF11 | 46610 | cross-linking aa 5-7 | + | + | | + | sta, 76% | | | | |
| ORF12 | 44026 | cross-linking aa 4-6 | + | + | + | + | cep, 84% | | | | |
| ORF13 | 42515 | cross-linking aa 1-3 | | | | + | sta, 75% | | | | |
| ORF14 | 43603 | cross-linking aa 2-4 | + | + | + | + | bal, 73% | | | | |
| ORF15 | 7714 | Unknown | + | + | + | + | cep, 88% | | | | |
| ORF16 | 200022 | NRPS, module 7 | + | + | + | + | cep, 78% | | | | |
| ORF17 | 433671 | NRPS, modules 4-6 | + | + | + | + | sta, 76% | | | | |
| ORF18 | 79133 | ABC transporter | | | | | | CAB | 4e−58 | S. coelicolor, ABC | |
| | | | | | | | | 89462.1 | | transporter | |
| ORF19 | 24733 | ABC transporter | | | | | | CAB | 3e−67 | S. coelicolor, ABC | |
| | | | | | | | | 89461.1 | | transporter | |
| ORF20 | 57418 | mannosyltransferase | | | | | | CAC | 6e−59 | S. coelicolor, | protein |
| | | | | | | | | 32663.1 | | unknown | mannosyl |
| | | | | | | | | | | | transferase |
| ORF21 | 29785 | Unknown | + | + | | | bal, 60% | | | | |
| ORF22 | 45887 | transmembrane | | | * | * | com, 54% | T30222 | 2e−49 | S. hygroscopicus, | |
| | | histidine kinase | | | | | | | | sensor protein | |
| | | | | | | | | | | kinase | |
| ORF23 | 74849 | Acyltransferase | | | | | | NP_103545.1 | 1e−58 | M. loti, probable | acyl- |
| | | | | | | | | | | acyltransferase | transferase |
| | | | | | | | | | | | 3 family |
| ORF24 | 69894 | ABC transporter | | + | + | + | sta, 76% | | | | |
| ORF25 | 221820 | NRPS, modules 1-2 | + | + | + | + | sta, 74% | | | | |
| ORF26 | 113832 | NRPS, module 3 | + | + | + | + | sta, 74% | | | | |
| ORF27 | 30307 | Methyltransferase | + | + | | | cep, 58% | | | | |
| ORF28 | 59291 | β-hydroxylase | | | | + | sta, 81% | | | | |
| ORF29 | 56959 | hexose oxidase | | | | | | NP_630371.1 | e−126 | S. coelicolor, | |
| | | | | | | | | | | putative secreted | |
| | | | | | | | | | | oxidoreductase | |
| ORF30 | 16502 | Unknown | | | | | | NP_626911.1 | 2e−18 | S. coelicolor, | 4-hydroxy |
| | | | | | | | | | | hypothetical | benzoyl-CoA |
| | | | | | | | | | | | thioesterase |
| ORF31 | 39458 | 3,5-dihydroxyphenyl | + | + | | + | bal, 89% | | | | |
| | | acetyl-CoA synthase | | | | | | | | | |
| ORF32 | 22178 | enhances activity of | + | + | | + | bal, 77% | | | | |
| | | 3,5-dihydroxyphenyl | | | | | | | | | |
| | | acetyl-CoA synthase | | | | | | | | | |
| ORF33 | 47840 | 3,5-dihydroxyphenyl | + | + | | + | bal, 82% | | | | |
| | | acetyl-CoA oxigenase | | | | | | | | | |
| ORF34 | 29396 | enhances activity of | + | + | | + | cep, 88% | | | | |
| | | 3,5-dihydroxyphenyl | | | | | | | | | |
| | | acetyl-CoA synthase | | | | | | | | | |
| ORF35 | 44118 | integral membrane | + | + | + | + | bal, 60% | | | | |
| | | ion transporter | | | | | | | | | |
| ORF36 | 26133 | type II thioesterase | | | | | | AAG | 2e−25 | A. mediterranei | |
| | | | | | | | | 52991.1 | | RifR, thioesterase | |
| ORF37 | 46605 | Aminotransferase | + | + | + | + | cep, 79% | | | | |

^{a}The + sign indicates the presence of an ortholog in other described glycopeptide gene clusters
^{b}When no orthologs are present in other glycopeptide gene clusters, the results on Blast searches in GeneBank are reported
^{c}Proposed function of the dbv ORF on the basis of the combined results from the presence in other glycopeptide clusters and Blast searches in GeneBank
^{d}This column reports the percent sequence identity of the best match from other glycopeptide gene clusters and the clusters it originates
^{e}Accession number of the GeneBank entry with the highest score
^{f} Probability score obtained from Blast searches
^{g}Organism and proposed function of the GeneBank entry from the previous column. Abbreviations are: S., Streptomyces; M., Mesorhizobium; A., Amycolatopsis
^{h}Conserved domains reported by Blast searches
* Present in other glycopeptide clusters, but sequences with higher identity present elsewhere in the database

Indeed, the genes encoding the seven modules of NRPS are organized as two divergently transcribed regions, separated by a 12-kb segment (FIG. 2). This contrasts with the organizations of the bal, cep, com and sta clusters, where the seven modules of NRPS genes are present in a compact region and translated all in the same direction. Furthermore, while in the bal, cep, com and sta clusters all ORFs except one are transcribed in the same direction, only 22 of the 37 dbv ORFs are transcribed in one direction, while the remaining 15 are transcribed in the opposite direction. This indicates a transcriptional complexity of the dbv cluster.

The dbv cluster is also characterized by the presence of several ORFs that do not find homologs in the bal, cep, com and sta clusters. These include dbv ORFs 3, 6 through 8, 18 through 20, 22, 23, 29, 30 and 36 (SEQ ID NOS: 4, 7 through 9, 19 through 21, 23, 24, 30, 31 and 37). A comparison among the five bal, cep, com, sta and dbv clusters is summarized in Table 1. In conclusion, the genetic organization of the dbv cluster as described herein is substantially different from those of other clusters involved in the synthesis of other glycopeptides. It therefore represents the first example of a cluster with such a genetic organization.

### B. Roles of the dbv Genes

The present invention discloses, in particular, the DNA sequence encoding the NRPS responsible for the synthesis of the heptapeptide precursor of A40926. The dbv NRPS consists of four polypeptides, each containing between 1 and 3 modules. These are designated dbv ORF16, ORF17, ORF25 and ORF26 (SEQ ID NOS: 17, 18, 26 and 27). Peptide synthesis by NRPSs is carried out by modular systems, where a loading module is followed by a series of elongating modules. In NRPSs, each elongating module is characterized by the presence of at least three domains: an adenylation (A) domain, responsible for substrate recognition and activation; a thiolation (T) domain, which covalently binds as thioesters amino acids and elongating peptides; and a condensation (C) domain, which catalyzes peptide bond formation. In addition to these core domains, the last module contains a thioesterase (Te) domain, which hydrolyzes the ester bond linking the completed peptide to the NRPS. Some modules convert an L-amino acid into the D-form through the action of an epimerization (E) domain. The dbv NRPS consists of seven modules, for a total of seven A domains, seven T domains, six C domains, three E domains and one Te domain. Specifically, dbv ORF26 (SEQ ID NO: 27) encodes NRPS modules 1 and 2, specifies the sequence of domains A-T-C-A-E-T and is required for the incorporation of a HPG and a Tyr residue (first two amino acids) in the heptapeptide core of A40926; dbv ORF25 (SEQ ID NO: 26) encodes NRPS module 3, specifies the sequence of domains C-A-T and is responsible for incorporating a DPG residue; dbv ORF17 (SEQ ID NO: 18) encodes NRPS modules 4 through 6, specifies the sequence of domains C-A-E-T-C-A-E-T-C-A-T and is responsible for incorporating two HPG and a Tyr residue in the A40926 heptapeptide core; and dbv ORF16 (SEQ ID NO: 17) encodes NRPS module 7, specifies the sequence of domains C-A-T-C*-T-Te (C* denotes an a typical condensation domain of unknown function) and is required for incorporation of the last DPG residue and in the release of the heptapeptide precursor of A40926.

Other genes present in the dbv cluster represent novel genetic elements useful for increasing production of A40926 or for synthesizing novel metabolites. Among these, dbv ORF9 (SEQ ID NO: 10) encodes the glycosyltransferase that attaches an N-acyl-glucosamine residue to the phenolic hydroxyl of the HPG residue at position 4 in the heptapeptide (Formula I). This gene can be cloned and expressed in a heterologous host to yield an active enzyme capable of attaching an N-acyl-glucosamine residue to other glycopeptide aglycones. Alternatively, dbv ORF9 can be inactivated in the producing strain, resulting in the formation of the A40926 aglycone. While this aglycone can be obtained by chemical means (Malabarba and Ciabatti 2001), it may be desirable to produce it through a single fermentation process, without the need for chemical intervention.

Yet other preferred nucleic acid molecules of the present invention include dbv ORF10 (SEQ ID NO: 11) that encodes a halogenase, responsible for the addition of chorine atoms at amino acid 3 and amino acid 6 of A40926. dbv ORF10 represents a novel genetic element, different from the halogenase genes present in the cep, com, sta and bal clusters. In fact, the A40926 chlorination pattern is rather unique among these glycopeptides. This gene can be cloned and expressed in a heterologous host to yield an active enzyme capable of chlorinating aromatic residues 3 and 6 of glycopeptides.

Yet other preferred nucleic acid molecules of the present invention include dbv ORF23 (SEQ ID NO: 24) that encodes an acyltransferase, responsible for N-acylation with a fatty acid of the glucosamine residue at amino acid 4. dbv ORF23 represents a novel genetic element, absent from the cep, com, sta and bal clusters. This gene can be cloned and expressed in a heterologous host to yield an active enzyme capable of N-acylating sugar moieties of different glycopeptides.

Yet other preferred nucleic acid molecules of the present invention include dbv ORF29 (SEQ ID NO: 30) that encodes a hexose oxidase, responsible for the oxidation to amino glucuronic acid of the D-glucosamine residue attached to amino acid 4 in A40926. dbv ORF29 represents a novel genetic element, absent from the cep, com, sta and bal clusters. This gene can be cloned and expressed in, a heterologous host to yield an active enzyme capable of oxidizing D-glucosamine residues attached to a glycopeptide.

Yet other preferred nucleic acid molecules of the present invention include dbv ORF36 (SEQ ID NO: 37) that encodes a thioesterase, responsible for hydrolyzing aberrant intermediate peptides from the NRPS. Similarly to other thioesterases present as a polypeptide distinct from the NRPS (Kotowska et al. 2002), the product of dbv ORF36 is responsible for maintaining an efficient NRPS for A40926 biosynthesis, by hydrolyzing all those thioesters on the NRPS that are not processed further into heptapeptides. It thus represents a novel genetic element, absent from the cep, sta, com and bal clusters. This gene can be cloned and expressed in another glycopeptide producer strain to increase the yield of product formed. Host strains include but are not limited to strains belonging to the order Actinomycetales, to the families Streptosporangiaceae, Micromonosporaceae, Pseudonocardiaceae and Streptomycetaceae, to the genera Nonomureae, Actinoplanes, Amycolatopsis, Streptomyces and the like.

Yet other preferred nucleic acid molecules of the present invention include dbv ORF20 (SEQ ID NO: 21) that encodes a mannosyltransferase, responsible for attaching a mannosyl residue to amino acid 7. It thus represents a novel genetic element, absent from the cep, sta, com and bal clusters. This gene can be cloned and expressed in another glycopeptide producer strain to yield glycopeptides carrying a mannosyl residue attached to amino acid 7. Alternatively, dbv ORF20 can be inactivated in the producing strain, resulting in the formation of demannosyl-A40926. While this compound an be obtained by other means (Lancini and Cavalleri 1997), it may be desirable to produce it through a single fermentation process.

The dbv cluster also includes a number of genes responsible for the synthesis of the non-proteinogenic amino acids HPG and DPG. For the synthesis of the former, the products of dbv ORFs 1, 2, 5 and 37 (SEQ ID NOS: 2, 3, 6 and 38) are required. Synthesis of DPG requires the participation of dbv ORFs 31 to 34 (SEQ ID NOS: 32 to 35), in addition to ORF37 (SEQ ID NO: 38). Their roles are summarized in Table 1. Since HPG and DPG are non-proteinogenic amino acids, synthesis of the heptapeptide by the NRPS depends on their availability. Consequently, the activity of these enzymes is a limiting step in glycopeptide biosynthesis. Increased yield of glycopeptides can thus be obtained by increasing the expression of these ORFs. These genes can be overexpressed, individually or in any combination of them, in the A40926 producing strain to increase the yield of A40926.

The dbv cluster also includes a number of genes responsible for exporting glycopeptide intermediates or finished products out of the cytoplasm and for conferring resistance to the producer cell. These genes include dbv ORFs 7, 18 to 19, 24 and 35 (SEQ ID NOS: 8, 19 to 20, 25 and 36). dbv ORF7 encodes a carboxypeptidase responsible for removing the terminal D-alanine moiety from the growing peptidoglycan. It represents a novel genetic element, absent from the cep, com, sta and bal clusters. dbv ORFs 18 to 19 and 24 encode transporters of the ABC class (van Veen and Konings 1998), responsible for the ATP-dependent excretion of A40926 or its intermediates. dbv ORF35 encodes an Na/K ion-antiporter, responsible for exporting A40926 or its intermediates against a proton gradient. These genes can be cloned and expressed, either individually or in any combination of them, in another glycopeptide producer strain to increase the yield of product formed. Host strains include but are not limited to strains belonging to the order Actinomycetales, to the families Streptosporangiaceae, Micromonosporaceae, Pseudonocardiaceae and Streptomycetaceae, to the genera Nonomureae, Actinoplanes, Amycolatopsis, Streptomyces and the like. Alternatively, these genes can be overexpressed, individually or in any combination of them, in the A40926 producing strain to increase the yield of A40926.

The dbv cluster also includes a number of regulatory genes, responsible or activating, directly or indirectly, the expression of biosynthetic and resistance genes during A40926 production. These genes include dbv ORFs 3, 4, 6 and 22 (SEQ ID NOS: 4, 5, 7 and 23). dbv ORF3 is highly related to HygR, a positive regulator present in a gene cluster from Streptomyces hygroscopicus (Ruan et al. 1997). It represents a novel genetic element, absent from the cep, com, bal and sta clusters. dbv ORF4 is highly related to similar regulators present in other glycopeptide clusters. dbv ORFs 6 and 22 together encode a two-component signal transduction system. These four genes can be cloned and expressed, either individually or in any combination of them, in another glycopeptide producer strain to increase the yield of product formed. Host strains include but are not limited to strains belonging to the order Actinomycetales, to the families Streptosporangiaceae, Micromonosporaceae, Pseudonocardiaceae and Streptomycetaceae, to the genera Nonomureae, Actinoplanes, Amycolatopsis, Streptomyces and the like. Alternatively, these genes can be overexpressed, individually or in any combination of them, in the A40926 producing strain to increase the yield of A40926.

### C. Uses of the dbv Cluster

The present invention provides also nucleic acids for the expression of the entire A40926 molecule, any of its precursors or a derivative thereof. Such nucleic acids include isolated gene cluster(s) comprising ORFs encoding polypeptides sufficient to direct the assembly of A40926. In one example, the entire dbv cluster (SEQ ID NO: 1) can be introduced into a suitable vector and used to transform a desired production host. In one aspect, this DNA segment is introduced into a suitable vector capable of carrying large DNA segments. Examples of such vectors include but are not limited to Bacterial Artificial Chromosome (BAC) vectors or specialized derivatives such as ESAC vectors (Shizuya et al. 1992; Ioannou et al. 1994; Sosio et al. 2000b). In another aspect, the dbv cluster is cloned as two separate segments into two distinct vectors, which can be compatible in the desired production host. In yet another aspect, the dbv cluster can be subdivided into three segments, each cloned into a separate, compatible vector. Examples of the use of one-, two- or three-vector systems have been described in the literature (e.g. Xue et al. 1999).

Once the dbv cluster has been suitably cloned into one or more vectors, it can be introduced into a number of suitable production hosts, where production of glycopeptide antibiotics might occur with greater efficiency than in the native host. Preferred host cells are those of species or strains that can efficiently express actinomycetes genes. Such hosts include but are not limited to Actinomycetales, Streptosporangiaceae, Micromonosporaceae, Pseudonocardiaceae and Streptomycetaceae, Nonomuraea, Actinoplanes, Amycolatopsis and Streptomyces and the like. Alternatively, a second copy of the dbv cluster, cloned into one or more suitable vectors, can be introduced the A40926 producing strain, where the second copy of dbv genes will increase the yield of A40926.

The transfer of the producing capability to a well characterized host can substantially improve several portions of the process of lead optimization and development: the titer of the natural product in the producing strain can be more effectively increased; the purification of the natural product can be carried out in a known background of possible interfering activities; the composition of the complex can be more effectively controlled; altered derivatives of the natural product can be more effectively produced through manipulation of the fermentation conditions or by pathway engineering.

Alternatively, the biosynthetic gene cluster can be modified, inserted into a host cell and used to synthesize or chemically modify a wide variety of metabolites: for example the open reading frames can be re-ordered, modified and combined with other glycopeptide biosynthesis gene cluster.

Using the information provided herein, cloning and expression of A40926 nucleic acids can be accomplished using routine and well known methods.

In another possible use, selected ORFs from the dbv gene cluster are isolated and inactivated by the use of routine molecular biology techniques. The mutated ORF, cloned in a suitable vector containing DNA segments that flank said ORF in the Nonomuria sp. ATCC39727 chromosome, is introduced into said Nonomuria strain, where two double cross-over events of homologous recombination result in the inactivation of said ORF in the producer strain. This procedure is useful for the production of precursors or derivatives of A40926 in an efficient manner.

In another possible use, selected ORFs from the dbv gene cluster are isolated and placed under the control of a desirable promoter. The engineered ORF, cloned in a suitable vector, is then introduced into Nonomuria sp. ATCC 39727, either by replacing the original ORF as described above, or as an additional copy of said ORF. This procedure is useful for increasing or decreasing the expression level of ORFs that are critical for production of the A40926 molecule, precursors or derivatives thereof.

### EXAMPLES

The following examples serve to illustrate the principles and methodologies through which the A40926 gene cluster is identified and the principles and methodologies through which all the dbv genes are identified and analyzed. These examples serve to illustrate the principles and methodologies of the present invention, but are not meant to limit its scope.

### General Methods

Unless otherwise indicated, bacterial strains and cloning vectors can all be obtained from public collections or commercial sources. Standard procedures are used for molecular biology (e.g. Sambrook et al. 1989; Kieser et al. 2000). Nonomuria was grown in HT agar (Kieser et al. 2000) and in Rare3 medium (10 g/l glucose, 4 g/l yeast extract, 10 g/1 malt extract, 2 g/l peptone, 2 g/l MgCl₂, 0.5% glycerol). Glycopeptides are isolated following published procedures (Lancini and Cavalleri, 1997). Sequence analyses are performed using the programs from the Wisconsin package, version 9.1 (Accelrys). Database searches are performed at with Blast or Fasta programs at public sites (http://www.ncbi.nlm.nih.gov/blast/index.html and http://www.ebi.ac.uk/fasta33).

### Example 1

### Isolation of A40926 Biosynthesis Genes

A genomic library is made with DNA from Nonomuria ATCC39727 in the cosmid vector Supercos (Stratagene, La Jolla, Calif. 92037). Total DNA from Nonomuria ATCC39727 was partially digested with Sau3AI in order to optimize fragment sizes in the 40 kb range. The partially digested DNA was treated with alkaline phosphatase and ligated to Supercos previously digested with BamHI. The ligation mixture was packaged in vitro and used to transfect E. coli XL1Blue cells. The resulting cosmid library was screened by hybridization with two probes obtained from PCR amplification of segments from the bal cluster using A. mediterranei DSM 5908 genomic DNA as template. These probes were: bgtfA, obtained from amplification with oligos 5′-ATGCGCGTGTTGATCTCG-3′ (SEQ ID NO: 39) and 5′-CGGCTGACCGCGGCGAAC-3′ (SEQ ID NO: 40); and dpgA, obtained from amplification with oligos 5′-CGTGGGGGTG GATGTATCGA-3′ (SEQ ID NO: 41) and 5′-TCACCATTGGATCAGCG-3′ (SEQ ID NO: 42). All oligos were designed from the sequence deposited in GenBank with accession No. Y16952. Further hybridization was performed with the oligonucleotide Pep8 (Sosio et al. 2000a). The cosmids positive to one or more of these probes were isolated and physically mapped with restriction enzymes. From such experiments, the cosmids reported in FIG. 1 were identified. The segment thus identified from the genome of Nonomuria sp. ATCC39727 contains the dbv gene cluster responsible for the synthesis of the antibiotic A40926.

The above example serves to illustrate the principle and methodologies through which the dbv cluster can be isolated. It will occur to those skilled in the art that the dbv cluster can be cloned in a variety of vectors. However, those skilled in the art understand that, given the 72-kb size of the dbv cluster, preferred vectors are those capable of carrying large inserts, such as lambda, cosmid and BAC vectors. Those skilled in the art understand that other probes can be used to identify the dbv cluster from such a library. From the sequence reported in SEQ ID NO: 1, any fragment can be PCR-amplified from Nonomuria sp. ATCC39727 DNA and used to screen a library made with such DNA. One or more clones from said library can be identified that includes any segment covered by SEQ ID NO: 1. Furthermore, it is also possible to identify the dbv cluster through the use of heterologous probes, such as those derived from the cep, bal, com and sta cluster, using the information provided in Table 1. Alternatively, other gene clusters directing the synthesis of secondary metabolites contain genes sufficiently related to the dbv genes as to allow heterologous hybridizations. All these variations fall within the scope of the present invention.

### Example 2

### Sequence Analysis of A40926 Gene Cluster

The dbv cluster, identified as described under Example 1, was sequenced by the shotgun approach. The sequence of the dbv cluster is provided herein as SEQ ID NO: 1. The resulting DNA sequence was analyzed with Codonpreference [GCG, (Genetic Computer group, Madison, Wis. 53711) version 9.1] to identify likely coding sequences. Next, each coding sequence identified in this way was analyzed by comparison against the bal, cep, com and sta clusters using the program Tfasta (GCG, version 9.1). Coding sequences not identifying matches in any of these clusters were then searched against GenBank, employing the programs Blast, or against SwissProt, using Fasta. Finally, the exact start codon for each ORF was established by multiple alignment of related sequences with the program Pileup (GCG, version 9.1) or by searching for an upstream ribosomal binding site. In total, 37 ORFs, denominated dbvORF1 through dbv ORF37, are identified. The results of these analyses are summarized in Table 1, and provided herein in the sequence listing as SEQ ID No: 2 through SEQ ID No: 38. Details are given below.

### 2A. Synthesis of Specialized Amino Acids HPG and DPG

Seven proteins encoded by the dbv cluster participate in the synthesis of the specialized amino acids HPG and DPG. Namely, ORF1 and ORF2 (SEQ ID NOS: 2 and 3) are involved in the synthesis of the HPG residues required for A40926 formation and they encode the p-hydroxymandelate oxidase and the p-hydroxymandelate synthetase, respectively. Homologs of these ORFs are found in other glycopeptide clusters (Table 1) and their roles have been established experimentally (Li et al. 2001; Hubbard et al. 2000). ORFs 31 to 34 (SEQ ID NOS: 32 to 35) are involved in the synthesis of the DPG residues required for A40926 formation. Homologs of these ORFs are found in other glycopeptide clusters that direct the synthesis of heptapeptide containing DPG residues (Table 1) and the involvement of the corresponding gene products has been determined experimentally (Pfeifer et al. 2001; Chen et al. 2001). ORF37 (SEQ ID NO: 38) encodes the amino transferase required for the transamination of both p-hydroxyphenylglyoxylate and 3,5-dihydroxyphenylglyoxylate, to yield HPG and DPG, respectively. Its role has been experimentally established (Pfeifer et al. 2001; Hubbard et al. 2000), and it utilizes preferentially tyrosine as an amino donor (Hubbard et al. 2000). This reaction results in the formation of p-hydroxyphenylpyruvate, which can then be converted into p-hydroxymandelate by the action of the gene product of ORF2 (SEQ ID NO: 3).

Other ORFs participating indirectly in the synthesis of HPG and DPG are also found in the dbv cluster, namely ORF5 and ORF 30 (SEQ ID NOS: 6 and 31). ORF5 (SEQ ID NO: 6) encodes a prephenate dehydrogenase that participates in the synthesis of p-hydroxyphenylpyruvate, the substrate for the product of ORF2 (SEQ ID NO: 3). This ORF therefore encodes the enzyme that primes the cycle converting tyrosine into HPG. The expression level of this ORF is therefore important in supplying adequate levels of HPG for A40926 formation. ORF30 (SEQ ID NO: 31) encodes a polypeptide highly similar to hypothetical polypeptides of unknown function identified from bacterial genome sequences, with the best matches being represented by NP_{—}626911.1 from S. coelicolor (Table 1). However, all these proteins display the conserved domain typical of 4-hydroxybenzoyl-CoA thioesterases (Benning et al. 1998). Thus, the product of ORF30 (SEQ ID No: 31) is likely to facilitate the release of DPG or one of its precursors during synthesis of this small polyketide. ORF30 (SEQ ID NO: 31) is unique to the dbv cluster (Table 1).

### 2B. Synthesis of the Heptapeptide Precursor of A40926

Four proteins, encoded by ORFs 16, 17, 25 and 26 (SEQ ID NOS: 17, 18, 26 and 27) are involved in the synthesis of the heptapeptide core of A40926. All of these show significant similarity to other NRPS. Based on alignments with other NRPS systems, the proposed domain composition and specificities of the proteins encoded by these four ORFs are reported in Table 2.

TABLE 2
Domain composition and roles of dbv NRPS

| dbv ORF | modules | domains | Amino acids | peptide bonds |
|---|---|---|---|---|
| ORF25 | 1-2 | AT-CATE | HPG, Tyr | 1-2 |
| ORF26 | 3 | CAT | DPG | 2-3 |
| ORF17 | 4-6 | CATE-CATE- | HPG, HPG, | 3-4, 4-5, |
| | | CAT | Tyr | 5-6 |
| ORF16 | 7 | CATC*Te | DPG | 6-7 |

The assignment of the specific roles of the dbv NRPS genes could not be predicted by their genetic localization within the dbv cluster. In fact, while for all the glycopeptide clusters reported thus far there is a colinearity between the genetic order of the modules and the order in which the corresponding amino acids are incorporated into the polypeptide, this is not the case for the dbv cluster (FIG. 2), since its NRPS genes are divergently transcribed. However, their roles and specificities can be predicted on the basis of the following observations:
i) the domain composition of the protein specified by ORF16 (SEQ ID NO: 17), and the fact that it terminates with a thioesterase domain, is most consistent with a role in recognition of a DPG residue and formation of the last peptide bond of the heptapeptide, followed by cleavage of the enzyme bound thioester (Table 2);
ii) the module organization and domain composition of ORF 17 (SEQ ID NO: 18) is most consistent with this polypeptide containing modules 4 to 6, required for recognizing amino acids 4 to 6 of the heptapeptide and for their incorporation, as seen with other glycopeptide NRPS systems (van Wageningen et al 1998; Pelzer et al. 1999; Chiu et al. 2001; Pootoolal et al. 2002);
iii) the domain organization of the product of ORF25 (SEQ ID NO: 26) is most consistent with its role in starting heptapeptide synthesis and catalyzing formation of the first peptide bond, since this ORF encodes two NRPS modules but just one C domain (Table 2);
iv) the domain organization of ORF26 (SEQ ID NO: 27) is most consistent with this polypeptide containing module 3, responsible for the recognition and incorporation of the third amino acid in the heptapeptide, since this module does not contain an E domain (required by the role of modules 2, 4 and 5) and the presence and absence of a C and a Te domain, respectively (Table 2), excludes that this ORF encodes modules 1 and 7, respectively.

Other ORFs participating indirectly in the synthesis of the heptapeptide precursor of A40926 are also found in the dbv cluster, namely ORF15 and ORF36 (SEQ ID NOS: 16 and 37). ORF15 (SEQ ID NO: 16) encodes a short peptide of unknown function. Homologs of this gene product are found in many clusters encoding NRPS systems. ORF36 (SEQ ID NO: 37) encodes a type II thioesterase, a protein often encoded by other clusters containing NRPS or polyketide synthase genes. The proposed role for these thioesterases is to enhance the efficiency by which NRPS and PKS systems operate, by removing aberrant intermediates covalently attached to the enzymes (Heathcote et al. 2001). No orthologs of this protein are encoded by the other known glycopeptide clusters (Table 1).

### 2C. Cross-Linking of the Aromatic Residues in the Heptapeptide

Four proteins, encoded by ORFs 11 through 14 (SEQ ID NOS: 12 through 15) are involved in the cross-linking reactions that join together the aromatic residues of the A40926 heptapeptide precursors. These four proteins show significant homologies to P450 monooxygenases (Table 1). On the basis of the level of identities with the P450 monooxygenases found in other glycopeptide clusters, and on the basis of the roles predicted for the P450 monooxygenases encoded by the genes present in the bal cluster (Bischoff et al. 2001), the following predictions can be made. Namely, the product of ORF 14 (SEQ ID NO: 15) is likely to be involved in the cross-linking of the aromatic residues of amino acids 2 and 4; the product of ORF 12 (SEQ ID NO: 13) is likely to be involved in the cross-linking of the aromatic residues of amino acids 4 and 6; and the product of ORF 11 (SEQ ID NO: 12) is likely to be involved in the cross-linking of the aromatic residues of amino acids 5 and 7. An ortholog of ORF 13 (SEQ ID NO: 14) is not present in the bal, cep and com clusters, but it is found in the sta cluster (Table 1). Since the structure of A47934, like that of A40926, contains an extra cross-link between the aromatic residues of amino acids 1 and 3, the product of ORF13 (SEQ ID NO: 14) is likely to be involved in this cross-linking reactions.

2D. Formation of β-hydroxytosine and Chlorination of Aromatic Residues

Two proteins, encoded by ORF10 and ORF28 (SEQ ID NOS: 11 and 29) are involved in the addition of a b-hydroxyl group to the tyrosine residue present as amino acid 6 in the heptapeptide and in the chlorination of the aromatic residues of amino acids 2 and 6. On the basis of the level of identities with the genes encoding halogenases found in other glycopeptide clusters, and on the basis of the roles predicted for the halogenase gene present in the bal cluster (Puk et al. 2002), the product of ORF 10 (SEQ ID NO: 11) is likely to be involved in the introduction of a chlorine atom into the aromatic residues of both amino acids 3 and 6. The product of ORF28 (SEQ ID NO: 29) is highly related a family of proteins that contain motifs typical of non-heme iron dioxygenases. One such protein is predicted from the sta cluster (Pootoolal et al. 2002) and is suggested to be involved in the b-hydroxylation of tyrosine. The exact timing of this hydroxylation reaction is not currently known. It could occur before incorporation of amino acid 6 into the heptapeptide, as it happens in the synthesis of balhimycin (Bischoff et al. 2001); it could occur during heptapeptide synthesis, or after completion of the heptapeptide skeleton.

### 2E. Addition and Modification of Sugars, and N-methylation

Five proteins, encoded by ORFs 9, 20, 23, 27 and 29 (SEQ ID NOS: 10, 21, 24, 28 and 30) are involved in some of the late steps in A40926 biosynthesis. Their predicted roles are as follows.

ORF9 (SEQ ID NO: 10) is highly related to proteins encoded by other glycopeptide clusters (Table 1), which have been demonstrated to be involved in the attachment of sugars to the p-hydroxyl group of the aromatic ring of the amino acid residue present at position 4 (Solenberg et al. 1997). Specifically, ORF9 (SEQ ID NO: 10) encodes a glycosyltransferase involved in the attachment of the N-acyl-glucosamine residue to the A40926 aglycone. No other glycosyltransferase with such a specificity is encoded by the other described glycopeptide clusters.

Homologs of ORF20 (SEQ ID NO: 21) are not found in the other described glycopeptide clusters. This protein contains motifs typical of the family of protein mannosyltransferases (Table 1). Furthermore, homologs of this ORF have been identified in the S. coelicolor genome (Table 1), as well as in the Actinoplanes spp. cluster specifying the synthesis of the antibiotic ramoplanin (WO0231155). Since ramoplanin contains a mannosyl residue attached to the peptide core, all these data point to a role for ORF20 (SEQ ID NO: 21) in attaching the mannosyl residue to the hydroxyl group of amino acid 7. This putative role is also demonstrated in Example 4 below.

Homologs of ORF23 (SEQ ID NO: 24) are not found in the other described glycopeptide clusters. This protein contains motifs typical of the family 3 of acyltransferases (Table 1). Since A40926 contains an acyl residue attached to the NH₂ group of the aminosugar residue, the product of this ORF is likely to be directly or indirectly involved in acylation of the A40926 precursor, resulting in the family of compounds that characterize the A40926 complex.

Homologs of ORF27 (SEQ ID NO: 28) are found in the bal and cep clusters (Table 1). It has been demonstrated that the homolog of ORF27 from the cep cluster is involved in the N-methylation of the terminal leucine residue of chloroeremomycin intermediates. An HPG residue is present at the N-terminal position in A40926. Consequently, the product of ORF27 (SEQ ID NO: 28) is likely to catalyze the N-methylation of an HPG residue in a glycopeptide precursor, and is thus endowed with a different specificity from the other described methyltransferases.

Homologs of ORF29 (SEQ ID NO: 30) are not found in other described glycopeptide clusters (Table 1). This protein contains motifs typical of FAD binding, and shows considerable matches to hexose oxidases (Table 1). Since A40926 contains a glucuronaminic residue attached to amino acid 4, the protein encoded by ORF29 (SEQ ID NO: 30) is likely to be involved in the oxidation of the glucosamine residue. Since this protein contains also a putative signal peptide sequence typical of proteins secreted out of the cytoplasm, it is likely that this oxidation occurs outside the cytoplasm, using as substrate a glucosamine residue attached to the glycopeptide core.

### 2F. Export and Resistance

Five proteins, encoded by ORFs 7, 18, 19, 24 and 35 (SEQ ID NOS: 8, 19, 20, 25 and 36) are involved in exporting A40926 or some of its precursor outside the cytoplasm and in conferring resistance to the producing strain. Their predicted roles are as follows.

Homologs of ORF7 (SEQ ID NO: 8) are not found in the other described glycopeptide clusters. This protein contains motifs typical of the VanY family of carboxypeptidases (Table 1). This family is best studied in some vancomycin-resistant enterococci, where it is involved in the removal of the terminal alanyl residue from some of the pentapeptide chains in nascent peptidoglycan, thus reducing the extent of glycopeptide binding to its molecular target (Evers et al. 1996). ORF7 (SEQ ID NO: 8) is therefore likely to be involved in conferring some level of resistance to A40926 in. the producing strain Nonomuria sp. ATCC38727.

Homologs of ORF24 and ORF35 (SEQ ID NOS: 25 and 36) are present in other glycopeptide clusters (Table 1). They are predicted to encode ABC-type and ion-dependent transmembrane transporters, respectively. They are thus likely to be involved in export or compartmentalization of A40926 or some of its precursors. Homologs of ORF18 and ORF19 (SEQ ID NOS: 19 and 20) are not found in other described glycopeptide clusters (Table 1). They are predicted to encode additional ABC-type transporters, and of these only ORF18 (SEQ ID NO: 19) is predicted to be a transmembrane protein. They are thus likely to be involved in export or compartmentalization of A40926 or some of its precursors.

### 2G. Regulation

Four proteins, encoded by ORFs 3, 4, 6 and 22 (SEQ ID NOS: 4, 5, 7 and 23) are involved in regulating the expression of one or more of the dbv genes. Homologs of ORF3 (SEQ ID NO: 4) are not found in the other described glycopeptide clusters. This protein contains motifs typical of positive regulators of the LuxR family, and is mostly related to one positive regulator found in a PKS cluster from Streptomyces hygroscopicus (Ruan et al. 1997). Homologs of ORF4 (SEQ ID NO: 5) are present in other glycopeptide clusters (Table 1), and belong to the family of LysR-type of positive transcriptional regulators. ORFs 3 and 4 (SEQ ID NOS: 4 and 5) are therefore likely to be required for the expression of one or more of the dbv genes. ORF6 and ORF22 (SEQ ID NOS: 7 and 23) encode the two members of a bacterial two-component signal transduction system. The former protein is a likely response regulators, with the best match found with the S. coelicolor CutR protein (Table 1). The latter protein is a likely transmembrane histidine kinase, mostly related to a putative sensor protein kinase from S. hygroscopicus (Table 1). ORFs 6 and 22 (SEQ ID NOS: 23) are therefore likely to be involved in sensing a signal that triggers the expression of one or more genes in the dbv cluster.

### Example 3

### Isolation of the dbv Cluster in an ESAC Vector

Using the information provided in Example 2, the dbv cluster was isolated in an ESAC vector as follows. A genomic library was made with DNA from Nonomuria ATCC39727 in the pPAC-S1 vector (Sosib et al. 2000b). DNA from Nonomuria ATCC39727 was prepared embedded in agarose plugs as described (Sosio et al. 2000b; WO99/67374), and partially digested with Sau3AI, in order to optimize fragment sizes in the 100-200 kb range. The resulting DNA fragments were briefly run on a PFGE gel, recovered and released from the agarose gel as described (Sosio et al. 2000b; WO99/67374). The resulting steps, including vector preparation, ligation and electroporation of E. coli DH10B competent cells, were performed as described (Sosio et al. 2000b; WO99/67374). The resulting colonies were arrayed onto nylon filters and screened by hybridization with two probes, PCR-amplified from Nonomuria ATCC39727 genomic DNA. Probe A was obtained using oligos 5′-TCAGGAGACGAACCCCGC-3′ (SEQ ID NO: 43) and 5′-GTGCACGAAAGTCCCGTC-3′ (SEQ ID NO: 44); and probe B with 5′-ATGGACTCCCACGTTCTC-3′ (SEQ ID NO: 45) and 5′-TCAGGGGAGACATGCGGT-3′ (SEQ ID NO: 46). All these sequences were derived from SEQ ID NO: 1. The ESAC clones positive to all these probes were then isolated and physically mapped by digestion with EcoRI and EcoRV. From one such experiment, the ESAC clone NmES1, containing an insert of about 84 kb, was isolated. NmES1 spans the entire dbv cluster (SEQ ID NO: 1) and extends it for about 5 kb 5′ to nucleotide 1 of SEQ ID NO: 1, and for about 8 kb 3′ to nt 71138 of SEQ ID NO: 1.

The above example serves to illustrate the principle and methodologies through which the dbv cluster can be obtained in an ESAC vector. It will occur to those skilled in the art that the vector pPAC-S1 is just one example of an ESAC vector that can be used for this purpose. Other vectors useful for cloning the entire dbv gene cluster and transferring into a suitable actinomycete host have been described (Sosio et al. 2000b; WO99/67374). Furthermore, other methods for preparing a large insert library of Nonomuria sp. ATCC39727 DNA, including but not limited to partial digestion, fragment separation and recovery, vector preparation, ligation and transformation of E. coli cells, also fall within the scope of the present invention. It will also occur to those skilled in the art that, once the boundaries of the dbv cluster are established as in SEQ ID NO: 1, any probe or probe combination other than probes A and B as described above, can be used to screen a library made with Nonomuria sp. ATCC39727 DNA to identify clones whose inserts span the entire dbv cluster. Alternatively, with the information provided in SEQ ID NO: 1 and in Table 1, other useful probes can be obtained from other gene clusters that contain genes sufficiently related to the dbv genes as to allow heterologous hybridizations. All these variations fall within the scope of the present invention.

### Example 4

### Manipulation of the A40926 Pathway by Gene Replacement

Using the information provided in Example 2, an in frame deletion in ORF 20 was constructed as follows. Fragment A was obtained through amplification with oligos 5′-TTTTGAATTCTCAGGCGATCCGTCCGTCT-3′ (SEQ ID NO: 47) and 5′-TTTTCTAGAGCCCGGACACCCGGGGGCTGA-3′ (SEQ ID NO: 48); and fragment B with oligos 5′-TTTTCTAGAAGTCATGGTGATGTGCGACAT-3′ (SEQ ID NO: 49) and 5′-TTTTAAGCTTATGTTGCAGGACGCCGACCG-3′ (SEQ ID NO: 50). Next, fragment A was digested with EcoRI and XbaI, fragment B with XbaI and HindIII, and both were ligated to pSET152 (Bierman et al. 1992) previously digested with EcoRI and HindIII. After transformation of E. coli DH5a cells, the resulting plasmid, designated pSM4, was recognized by the presence of fragments of 4 kb and 1.5 kb after digestion with EcoRI and HindIII. An aliquot of pSM4 was transferred into E. coli ET12567(pUB307) (Kieser et al. 2000) cells, yielding strain SM4. Then, about 10⁸ CFU of SM4 cells, from an overnight culture in LB, were mixed with about 10⁷ CFU of Nonomuria ATCC39727 grown in Rare3 medium for about 80 h. The resulting mixture was spread onto HT plates, which were then incubated at 28° C. for about 20 h. After removing excess E. coli cells with a gentle wash with water, plates were overlaid with 3 ml soft agar containing 200 mg nalidixic acid and 15 mg/ml apramycin. After further incubation at 28° C. for 3-5 weeks, Nonomuria ex-conjugants were streaked onto fresh medium containing apramycin. One such ex-conjugant, named strain SS18, was further processed. Strain SS18 was then grown for several passages in HT medium without apramycin and appropriate dilutions were plated on HT agar without apramycin. Individual colonies were then analyzed by PCR, using oligos 5′-TTTTGAATTCTCAGGCGATCCGTCCGTCT-3′ (SEQ ID NO: 47) and 5′-TTTTAAGCTTATGTTGCAGGACGCCGACCG-3′ (SEQ ID NO: 50). Colonies containing the deleted allele of ORF20 were recognized by the presence of a 1.5 kb band. One such colony; designated SSM18, was grown in HT medium and the formation of demannosyl-A40926 was confirmed by comparison with an authentic standard (Malabarba and Ciabatti 2001).

The above example serves to illustrate the principle and methodologies through which an ORF chosen among any of those specified by SEQ ID NOS: 2 to 38 can be replaced by a mutated copy in the A40926 producing strain Nonomuria sp. ATCC39727. It will occur to those skilled in the art that ORF20 (SEQ ID NO: 21) is just an example of the methodologies for creating in frame deletions in the cluster specified by SEQ ID NO: 1. Those skilled in the art understand also that in frame-deletions are just one method for generating mutations, and that other methods including but not limited to frame-shift mutations, insertions and site-directed mutations can also be used to generate null mutants in any of the ORFs specified by SEQ ID NOS: 2 to 38. Those skilled in the art also understand that, having established a method for generating mutations in any of the ORFs specified by SEQ ID NOS: 1, these same methodologies can be applied for altering the expression levels of these same ORFs. Examples for how this can be achieved include but are not limited to integration of multiple copies of said ORFs into any place in the Nonomuria sp. ATCC39727 genome, alteration in the promoters controlling the expression of said ORFs, removal of antisense RNAs or transcription terminators interfering with their expression.

Finally, variations in the vectors used for introducing the mutated alleles into Nonomuria sp. ATCC39727, in the conditions for conjugation and cultivation of the donor and recipient strain, in the method for selecting and screening ex-conjugants and their derivatives, all fall within the scope of the present invention.

### REFERENCES

Bate N, Butler A R, Gandecha A R, Cundliffe E (1999) Chem Biol 6: 617-624.
Benning M M, Wesenberg G, Liu R, Taylor K L, Dunaway-Mariano D, Holden H M (1998) J Biol Chem 273: 33572-33579.
Bentley S D, Chater K F, Cerdeno-Tarraga A M, Challis G L, Thomson N R, James K D, Harris D E, Quail M A, Kieser H, Harper D, Bateman A, Brown S, Chandra G, Chen C W, Collins M, Cronin A, Fraser A, Goble A, Hidalgo J, Hornsby T, Howarth S, Huang C H, Kieser T, Larke L, Murphy L, Oliver K, O'Neil S, Rabbinowitsch E, Rajandream M A, Rutherford K, Rutter S, Seeger K, Saunders D, Sharp S, Squares R, Squares S, Taylor K, Warren T, Wietzorrek A, Woodwardm J, Barrell B G, Parkhill J, Hopwood D A (2002) Nature 417: 141-147.
Bierman R, Logan K, O'Brien E T, Seno R, Nagaraja R, Schoner B E (1992) Gene 116: 43-49.
Bischoff D, Pelzer S, Holtzel A, Nicholson G J, Stockert S, Wohlleben W, Jung G, Sussmuth R D (2001) Angew Chem Int Ed Engl 40: 1693-1696.
Chater K F, Bibb M (1997) in Biotechnology, vol 6, pp. 57-105, VCH, Weinheim, Germany.
(Kleinkauf H, von Dohren H eds), VCH, Weinheim, Germany.
Chen H, Tseng C C, Hubbard B K, Walsh C T (2001) Proc Natl Acad Sci USA 98: 14901-14906.
Chiu H T, Hubbard B K, Shah A N, Eide J, Fredenburg R A, Walsh C T, Khosla C (2001) Proc Natl Acad Sci USA 98: 8548-8553.
Evers S, Quintiliani R Jr, Courvalin P (1996) Microb Drug Resist 2: 219-223.
Heathcote M L, Staunton J, Leadlay P F (2001) Chem Biol 8: 207-220.
Hubbard B K, Thomas M G, Walsh C T (2000) Chem Biol 7: 931-942.
Katz L, McDaniel R (1999) Med. Res. Rev. 19: 543-58.
Kieser T, Bibb M J, Buttner M J, Chater K F, Hopwood D A (2000) Practical Streptomyces Genetics, The John Innes Foundation, Norwich, UK.
Kotowska M, Pawlik K, Butler A R, Cundliffe E, Takano E, Kuczek K (2002) Microbiology 148: 1777-1883.
Ioannou P A. Amermiya C T, Garnes J, Kroisel P M, Shizuya H, Chen C, Batzer M A, de Jong P J (1994) Nat Genet. 6: 84-89.
Lancini G C, Cavalleri B (1990) In: Kleinkauf H, von Dohren H (eds), Biochemistry of Peptide Antibiotics pp. 159-178 Walter de Gruyter Berlin, New York.
Lancini G C, Cavalleri B (1997) Glycopeptide antibiotics (Dalbaaheptides) in Biotechnology, vol 7, pp. 369-396, VCH, Weinheim, Germany.
Li T L, Choroba O W, Hong H, Williams D H, Spencer J B (2001) Chem Commun 20: 2156-2157.
Losey H C, Peczuh M W, Chen Z, Eggert U S, Dong S D, Pelczer I, Kahne D, Walsh C T (2001) Biochemistry 40: 4745-4755.
Malabarba A, Ciabatti R (2001) Curr Med Chem 8: 1759-1773.
Malabarba A. Ciabatti R, Gerli E, Ripamonti F, Ferrari P, Colombo L, Olsufyeva E N, Pavlov A Y, Reznikova M I, Lazhko E I, Preobrazhenskaya M N (1997) J Antibiot 50: 70-81.
Marahiel M A (1997) Chem Biol 4: 561-577.
Mendez C, Salas J A. (2001) Trends Biotechnol 19: 449-456.
Omura S, Ikeda H, Ishikawa J, Hanamoto A, Takahashi C, Shinose M, Takahashi Y, Horikawa H, Nakazawa H, Osonoe T, Kikuchi H, Shiba T, Sakaki Y, Hattori M (2001) Proc Natl Acad Sci USA 98: 12215-12220.
Parenti F, Cavalleri B (1989) J Antibiot 42:1882-1883.
Parenti F, Cavalleri B (1990) Drugs of the future 15: 57-72.
Pelzer S, Sussmuth R, Heckmann D, Recktenwald J, Huber P, Jung G, Wohlleben W (1999) Antimicrob Agents Chemother 43: 1565-1573.
Pfeifer V, Nicholson G J, Ries J, Recktenwald J, Schefer A B, Shawky R M, Schroder J, Wohlleben W, Pelzer S (2001) J Biol Chem 276, 38370-38377.
Pootoolal J, Thomas M G, Marshall C G, Neu J M, Hubbard B K, Walsh C T, Wright G D (2002) Proc Natl Acad Sci USA 99: 8962-8967.
Puk O, Huber P, Bischoff D, Recktenwald J, Jung G, Sussmuth R D, van Pee K H, Wohlleben W, Pelzer S (2002) Chem Biol 9: 225-235.
Rodriguez L, Aguirrezabalaga I, Allende N, Brana A F, Mendez C, Salas J A (2002) Chem. Biol. 9:721-729.
Ruan X, Stassi D, Lax S A, Katz L (1997) Gene 203: 1-9.
Sambrook J, Fritsch E F, Maniatis T (1989) Molecular Cloning: A laboratory Manual (Cold Spring Harbor Lab. Press, Cold Spring Harbor N.Y.).
Shizuya H, Birren B, Kim U J, Mancino V, Slepak T, Tachiri Y, Simon M (1992) Proc Natl Acad Sci USA 89: 8794-8797.
Solenberg P J, Matsushima P, Stack D R, Wilkie S C, Thompson R C, Baltz R H (1997) Chem Biol 4:195-202.
Sosio M, Bossi E, Bianchi A, Donadio S (2000a) Mol Gen Genet 264: 213-221.
Sosio M, Giusino F, Cappellano C, Bossi E, Puglia A M, Donadio S (2000b) Nat Biotechnol 18: 343-345.
Steiert M, Schmitz F J (2002) Curr Opin Investig Drugs 3: 229-233.
van Wageningen A M, Kirkpatrick P N, Williams D H, Harris B R, Kershaw J K, Lennard N J, Jones M, Jones S J, Solenberg P J (1998) Chem Biol 5: 155-162.
van Veen H W, Konings W N (1998) Biochim Biophys Acta 1365: 31-36.
Xue Q, Ashley G, Hutchinson C R, Santi D V (1999) Proc Natl Acad Sci USA 96: 11740-11745.
Zhang Z, Wang Y. Ruan J (1998) Int J Syst Bacteriol 48:411-422.

1. An isolated nucleic acid comprising a nucleotide sequence selected from the group consisting of:
a) the dbv gene cluster encoding the polypeptides required for the synthesis of A40926 (SEQ ID NO: 1);
b) a nucleotide sequence encoding the same polypeptides encoded by the dbv gene cluster (SEQ ID NO: 1), other than the nucleotide sequence of the dbv gene cluster;
c) any nucleotide sequence of dbv ORFs 1 to 37, encoding the polypeptides of SEQ ID NOS: 2 to 38; and
d) a nucleotide sequence encoding the same polypeptides encoded by any of dbv ORFs 1 to 37 (SEQ ID NOS: 2 to 38), other than the nucleotide sequence of said ORF.

2. The isolated nucleic acid of claim 1, wherein the nucleotide sequence is selected from the group consisting of:
e) a nucleotide sequence of any of dbv ORFs 3 to 4, 6 to 10, 18 to 20, 22 to 23, 29 to 30, and 36 (SEQ ID NOS: 4 to 5, 7 to 11, 19 to 21, 23 to 24, 30 to 31, and 37);
f) a nucleotide sequence encoding the same polypeptide encoded by any of dbv ORFs 3 to 4, 6 to 10, 18 to 20, 22 to 23, 29 to 30, and 36 (SEQ ID NOS: 4 to 5, 7 to 11, 19 to 21, 23 to 24, 30 to 31, and 37), other than the nucleotide sequence of said ORF;
g) a nucleotide sequence encoding a polypeptide that is at least 80% identical in amino acid sequence to a polypeptide encoded by any of dbv ORFs 3, 6 to 9, 18 to 20, 22 to 23, 29 to 30, and 36 (SEQ ID NOS: 4, 7 to 10, 19 to 21, 23 to 24, 30 to 31, and 37); and
h) a nucleotide sequence encoding a polypeptide that is at least 87% identical in amino acid sequence to a polypeptide encoded by any of dbv ORFs 4 and 10 (SEQ ID NOS: 5 and 11).

3. The isolated nucleic acid according to claim 2, wherein the nucleic acid sequence comprises a combination of nucleotide sequences that encode polypeptides required for the synthesis of the 4-hydroxy-phenylglycine residues of A40926 selected from the group consisting of dbv ORFs 1, 2, 5, 37 (SEQ ID NOS: 2, 3, 6 and 38), and other nucleotide sequences encoding the same polypeptides.

4. The isolated nucleic acid according to claim 2, wherein the nucleic acid sequence comprises a combination of nucleotide sequences that encode polypeptides required for the synthesis of the 3,5-dihydroxy-phenylglycine residues of A40926 selected from the group consisting of dbv ORFs 30 to 34, 37 (SEQ ID NOS: 31 to 35, and 38), and other nucleotide sequences encoding the same polypeptides.

5. The isolated nucleic acid according to claim 2, wherein the nucleic acid sequence comprises a combination of nucleotide sequences that encode polypeptides required for the synthesis of the heptapeptide skeleton of A40926 selected from the group consisting of dbv ORFs 16, 17, 25, 26, 36 (SEQ ID NOS: 17 to 18, 26 to 27, and 37), and other nucleotide sequences encoding the same polypeptides.

6. The isolated nucleic acid according to claim 2, wherein the nucleic acid sequence comprises a nucleotide sequence that encodes a polypeptide required for the chlorination of the aromatic residues of amino acids 3 and 6 of A40926 selected from the group consisting of dbv ORF 10 (SEQ ID NO: 11), and other nucleotide sequences encoding the same polypeptide.

7. The isolated nucleic acid according to claim 2, wherein the nucleic acid sequence comprises a nucleotide sequence that encodes a polypeptide required for the β-hydroxylation of the tyrosine residue of amino acid 6 of A40926 selected from the group consisting of dbv ORF 28 (SEQ ID NO: 29), and other nucleotide sequences encoding the same polypeptide.

8. The isolated nucleic acid according to claim 2, wherein the nucleic acid sequence comprises a combination of nucleotide sequences that encode polypeptides required for the cross-linking of the aromatic residues of amino acids at positions 2 and 4, 4 and 6, 1 and 3, and 5 and 7 of A40926 selected from the group consisting of dbv ORFs 11 to 14 (SEQ ID NOS: 12 to 15), and other nucleotide sequences encoding the same polypeptides.

9. The isolated nucleic acid according to claim 2, wherein the nucleic acid sequence comprises a combination of nucleotide sequences that encode polypeptides required for the addition and formation of the N-acyl glucuronamine residue of A40926 selected from the group consisting of ORFs 9, 23, 29 (SEQ ID NOS: 10, 24 and 30), and other nucleotide sequences encoding the same polypeptides.

10. The isolated nucleic acid according to claim 2, wherein the nucleic acid sequence comprises a nucleotide sequence that encodes a polypeptide required for the attachment of the mannosyl residue of A40926 selected from the group consisting of dbv ORF 20 (SEQ ID NO: 21), and other nucleotide sequences encoding the same polypeptide.

11. The isolated nucleic acid according to claim 1, wherein the nucleic acid sequence comprises a nucleotide sequence that encodes a polypeptide required for the N-methylation of A40926 selected from the group consisting of dbv ORF 27 (SEQ ID NO: 28), and other nucleotide sequences encoding the same polypeptide.

12. The isolated nucleic acid according to claim 2, wherein the nucleic acid sequence comprises a combination of nucleotide sequences that encode polypeptides required for export of A40926 or some of its precursors outside of the cytoplasm and for conferring resistance to A40926 to the producing strain selected from the group consisting of dbv ORFs 7, 18, 19, 24, 35 (SEQ ID NOS: 8, 19 to 20, 25 and 36), and other nucleotide sequences encoding the same polypeptides.

13. The isolated nucleic acid according to claim 2, wherein the nucleic acid sequence comprises a combination of nucleotide sequences that encode polypeptides required for regulating the expression of one or more genes of the dbv gene cluster selected from the group consisting of dbv ORFs 3, 4, 6, 22 (SEQ ID NOS: 4, 5, 7 and 23), and other nucleotide sequences encoding the same polypeptides.

14. The isolated nucleic acid according to claim 1, wherein the nucleic acid sequence comprises a nucleotide sequence comprises the dbv gene cluster encoding the polypeptide required for the synthesis of a A40926, wherein an in frame deletion has been introduced in the nucleotide sequence encoding the polypeptides required for the attachment of the mannosyl residue.

15. The isolated nucleic acid according to claim 1 comprising a nucleotide sequence carrying at least one extra-copy of at least one of the dbv ORFs 1 to 37 (SEQ ID NOS: 2 to 38) or of a nucleotide sequence encoding the same polypeptides encoded by said dbv ORF, other than the nucleotide sequence of said dbv ORF.

16. (canceled)

17. A recombinant DNA vector which comprises a DNA sequence as selected from the group consisting of claim 1.

18. The recombinant vector according to claim 17, wherein the recombinant vector is an ESAC vector.

19. (canceled)

20. (canceled)

21. A method for increasing production of A40926 by a microorganism capable of producing A40926 or a precursor thereof by means of a biosynthetic pathway, said method comprising:
a) transforming with a recombinant DNA vector a microorganism that produces A40926 or a A40926 precursor by means of a biosynthetic pathway, wherein said DNA vector codes for the expression of an activity that is rate limiting in said pathway;
b) culturing said microorganism transformed with said vector under conditions suitable for cell growth, expression of said gene, and production of said antibiotic or antibiotic precursor.

22. (canceled)

23. (canceled)

24. A transformed A40926-producing microorganism having A40926 biosynthetic genes in its genome wherein at least one of the A40926 biosynthetic genes, selected from dbv ORFs 1 to 37 (SEQ ID NOS: 2 to 38), is disrupted.

25. The transformed microorganism according to claim 24, wherein the biosynthetic gene which is disrupted is the gene involved in the attachment of the mannosyl residue.

26. (canceled)

27. A method for producing a glycopeptide different from A40926 or a precursor thereof, comprising the steps of:
a) (i) transforming with a recombinant DNA vector a microorganism that produces a glycopeptide or a glycopeptide precursor different from A40926 or a precursor thereof by means of a biosynthetic pathway, said vector or portion thereof comprising one or more nucleotide sequences coding for the expression of one or more polypeptides that modify said glycopeptide or glycopeptide precursor; and
(ii) culturing said microorganism transformed with said vector under conditions suitable for cell growth, expression of said gene and production of said antibiotic or antibiotic precursor;
or
b) (i) transforming with a recombinant DNA vector a microorganism, said vector comprising one or more nucleotide sequences coding for one or more active polypeptides that modifies a glycopeptide or glycopeptide precursor, wherein said microorganism is selected from among those that do not produce glycopeptides or glycopeptide precursors and that can efficiently express the one or more nucleotide sequences;
(ii) preparing a cell extract or cell fraction of said microorganism under conditions suitable for the presence of the one or more active polypeptides, said cell extract or cell fraction containing at least said one or more active polypeptides; and
(iii) adding a glycopeptide or glycopeptide precursor to said cell extract or cell fraction to form a mixture; and
(iv) incubating said mixture under conditions where said one or more active polypeptides can modify said glycopeptide or glycopeptide precursor.

28. An isolated polypeptide comprising a polypeptide sequence involved in the biosynthetic pathway of A40926 selected from the group consisting of
a) an ORF polypeptide encoded by any of dbv ORFs 1 to 37 (SEQ ID NOS: 2 through 38) or a polypeptide which is identical in amino acid sequence to an ORF polypeptide encoded by any of dbv ORFs 1 to 37 (SEQ ID NOS: 2 through 38);
b) a polypeptide which is at least 80% identical in amino acid sequence to a polypeptide encoded by any of dbv ORFs 3, 6 to 9, 18 to 20, 22 to 23, 29 to 30 and 36 (SEQ ID NOS.: 4, 7 to 10, 19 to 21, 23 to 24, 30 to 31 and 37); and
c) a polypeptide which is at least 87 identical in amino acid sequence to a polypeptide encoded by any of dbv ORFs 4 and 10 (SEQ ID NOS: 5 and 11).

29. (canceled)
